(19) United States
(12) Patent Application Publication
(10) Pub. No.: US 2025/0122512 A1
(43) Pub. Date: Apr. 17, 2025

(54) CLEAVE AND RESCUE GAMETE KILLERS FOR GENE DRIVE IN PLANTS

(71) Applicant: California Institute of Technology, Pasadena, CA (US)

(72) Inventors: Georg Oberhofer, Pasadena, CA (US); Bruce A. Hay, Pasadena, CA (US)

(21) Appl. No.: 18/913,953

(22) Filed: Oct. 11, 2024

Related U.S. Application Data
(60) Provisional application No. 63/544,107, filed on Oct. 13, 2023.

(51) Int. Cl. C12N 15/82; C12N 9/22; C12N 15/11

(52) U.S. Cl. CPC C12N 15/8213; C12N 9/22; C12N 15/11; C12N 2310/20

(57) ABSTRACT

Described herein are embodiments relating to manipulation of the composition, numbers and sex ratio in plant populations through DNA sequence modifications.

### CROSS-REFERENCE TO RELATED APPLICATIONS

This application claims priority to U.S. Provisional Application No. 63/544,107, filed Oct. 13, 2023, the entire contents of each of which is incorporated by reference herein.

### STATEMENT REGARDING FEDERALLY SPONSORED R&D

This invention was made with government support from the US Department of Agriculture, National Institute of Food and Agriculture specialty crop initiative under USDA NIFA Award No. 2012-51181-20086. The government has certain rights in the invention.

### REFERENCE TO ELECTRONIC SEQUENCE LISTING

This application incorporates by reference the material contained in the Sequence Listing XML file being submitted concurrently herewith: File name: CALTE173A_ST26.xml; created on Oct. 10, 2024 and is 8192 bytes in size.

### BACKGROUND

### Field

The disclosure is generally related to DNA sequence modification-based modification of a population of organisms, such as plants.

### Description of the Related Art

Gene drive occurs when genetic elements-including genes, gene complexes, entire chromosomes and endosymbiotic bacteria—are transmitted to viable, fertile progeny at rates greater than those due to Mendelian transmission, resulting in an increase in their frequency in the population over time, even if their presence results in a fitness cost to carriers.

### SUMMARY

In some embodiments, methods for performing spread of a gene or multi-gene cassette in a population of an organism are provided. In some embodiments, performing a spread of a gene or a multi-gene cassette in a population of an organism refers to a situation in which genetic elements including alleles of specific genes, gene complexes, or entire chromosomes are transmitted to viable, fertile progeny at rates greater than those due to Mendelian transmission, resulting in an increase in their frequency in the population over time, even if their presence results in a fitness cost to carriers. In some embodiments the population is a population of plants. In some embodiments, the methods comprise obtaining an organism and introducing into the organism one or more vectors comprising a nucleic acid sequence encoding: a first gene encoding a rescue transgene; a first enhancer/promoter operably linked to a second gene encoding a DNA sequence modifying enzyme and optionally, one or more regulatory sequences. In some embodiments the DNA modifying enzyme modifies an endogenous copy of an essential gene, wherein the essential gene is essential for survival or fertilization ability of gametes from one or both sexes. The vector may comprise a second promoter operably linked to one or more guide RNAs (gRNAs) targeting the endogenous copy of an essential gene. In some embodiments the vector may comprise one or more cargo genes, encoding a cargo protein to be introduced into the population. The DNA sequence modifying enzyme is expressed in a cell or cells of the organism that ultimately gives rise to a germline, thereby inducing one or more sequence modifications in the essential gene in the cell, such that the essential gene is rendered partially or wholly non-functional. If the rescue gene is not present, the modification of the essential gene results in death of the cell or loss of ability to participate in fertilization of a gamete ell. Thus, in some embodiments a partially or wholly non-functional gamete phenotype is rescued by the rescue transgene, resulting in an altered gamete containing the one or more vectors. Progeny carrying the altered gamete can be introduced in an environment. In some embodiments, only the cells (gametes) that contain a vector with the rescue gene survive, thereby increasing the frequency of progeny bearing the vector relative to wild type in a population of the organism. In some embodiments, an increase in a frequency of the rescue transgene bearing progeny is desired relative to a frequency of a wild type individual organism in a population of the organism. In some embodiments, the vector encodes a cargo gene. Leading to expression of the cargo gene in the population of the organism.

In some embodiments, the one or more vectors are configured such that the second gene, the first enhancer/promoter, the second promoter and/or the nucleic acid encoding one or more gRNAs are positioned on a different chromosome or an extra-chromosomal element than the first gene.

In some embodiments, the one or more vectors are configured such that the second gene, the first enhancer/promoter, the second promoter and/or the nucleic acid encoding one or more gRNAs are positioned on the same chromosome at a distance from the first gene. In some embodiments, the distance is less than 50 map units. In some embodiments, the organism is a diploid that produces haploid gametes. In some embodiments, the diploid undergoes meiosis to form haploid gametes before undergoing fertilization to form a new diploid. In some embodiments, the progeny is formed by mating of haploid gametes of the same or two related species. In some embodiments, the organism is a polyploid that gives rise to gametes with half the adult number of each chromosome type.

In some embodiments, the organism is a plant. In some embodiments, the organism is a pest, weed, invasive species, or species of interest for agriculture, conservation or ecosystems services. In some embodiments, the gene spread renders the population of an organism vulnerable to toxicity in the presence of a chemical, compound or condition. In some embodiments, the chemical or compound is an herbicide, pesticide or environmental condition such as temperature, salinity, light or water levels. In some embodiments, the gene spread renders the population of an organism less harmful to other species in the environment. In some embodiments, the gene spread renders the population of an organism resistant to toxicity in the presence of a chemical, compound or environmental condition. In some embodiments, the condition is drought, stress, inadequate soil fertility, nutrient deficiencies, salinity, pollution, flooding, or presence of a pest, competitor, or pathogen.

In some embodiments, a method of performing a gene drive-mediated population suppression of an organism is provided, wherein one or more vectors is inserted into a gene required for diploid fertility of one sex, thereby disrupting the gene required for diploid fertility of one sex. In some embodiments, the method comprises obtaining an organism and introducing into the organism one or more vectors. In some embodiments, the one or more vectors are introduced into a gene required for diploid fertility. In some embodiments the one or more vectors comprise a nucleic acid sequence encoding: a first gene encoding a rescue transgene; a first enhancer/promoter operably linked to a second gene encoding a DNA sequence modifying enzyme and optionally, one or more regulatory sequences. In some embodiments the DNA modifying enzyme modifies an endogenous copy of an essential gene. In some embodiments the one or more vectors comprise a second promoter operably linked to nucleic acid sequences encoding one or more guide RNAs (gRNAs) targeting the endogenous copy of the essential gene. In some embodiments, the one or more vectors may comprise one or more cargo genes, encoding a protein to be expressed in the population. The one or more vectors is inserted into a gene required for diploid fertility of one sex, thereby disrupting the gene required for diploid fertility of one sex. The DNA sequence modifying enzyme is expressed in a cell of the organism that ultimately gives rise to a germline, inducing one or more sequence modifications in the essential gene in the cell, resulting in the essential gene being rendered partially or wholly non-functional, resulting in death or loss of ability to participate in fertilization if the cell lacks the rescue transgene. In some embodiments, the partially or wholly non-functional gamete is rescued by the rescue transgene. In some embodiments, an altered gamete containing one or more vectors is generated, wherein the altered gamete in which the essential gene has been rendered partially or wholly non-functional has been rescued by the rescue transgene. In some embodiments, only the cells (gametes) that contain a vector with the rescue gene survive, thereby increasing the frequency of progeny cells bearing vector relative to wild type in a population of the organism. The progeny carrying the altered gamete containing one or more vectors is introduced in an environment wherein an increase in the frequency of the rescue transgene bearing progeny is desired relative to a frequency of a wild organism in a population of the organism. In some embodiments, killing of gametes that fail to inherit the vector results in a relative increase in the frequency of those that do carry it. In some embodiments, the increase in frequency of the transgene-bearing progeny results in the creation of diploids or polyploids who are sterile in one sex due to the presence of two or more copies of the vector, which disrupt all copies of the gene essential for diploid or polyploid fertility of that sex. In some embodiments, the increase in sterile organisms leads to a decline in the population.

In some embodiments, the essential gene is required for gamete function. In some embodiments, the organism gives rise to a haploid gamete. In some embodiments, the organism is a diploid that produces haploid gametes. In some embodiments, the diploid adult undergoes meiosis to form haploid gametes before undergoing fertilization to form a new diploid. In some embodiments, the haploid gametes that do not include a vector with a rescue gene will die. In some embodiments, the progeny is formed by mating the haploid gametes with an organism of the same species. In some embodiments, the organism is a polyploid that gives rise to gametes with half the adult number of each chromosome type. In some embodiments, the organism is a plant.

In some embodiments, the one or more vectors is inserted next to or within a locus whose presence is sufficient for adult male or female sex determination. In some embodiments, the gene required for diploid fertility of one sex, is a gene for diploid female fertility. In some embodiments, the gene required for diploid fertility of one sex, is a gene for diploid male fertility. In some embodiments, the gene required for polyploid fertility of one sex, is a gene required for polyploid male fertility. In some embodiments, the gene required for polyploid fertility of one sex, is a gene required for polyploid female fertility.

In some embodiments, methods of gene drive-mediated population suppression of an organism are provided. In some embodiments, the method comprises obtaining the organism, introducing into the organism one or more vectors comprising a nucleic acid sequence encoding: a first gene encoding a rescue transgene; a first enhancer/promoter operably linked to a second gene encoding the DNA sequence modifying enzyme and optionally, one or more regulatory sequences. In some embodiments the DNA modifying enzyme modifies an endogenous copy of an essential gene. In some embodiments the one or more vectors additionally comprise a second promoter operably linked to one or more guide RNAs (gRNAs) targeting the endogenous copy of the essential gene. In some embodiments the one or more vectors optionally comprise one or more cargo genes to be expressed in the organism. The one or more vectors may be inserted next to or within a locus whose presence is sufficient for adult male or female sex determination. The DNA sequence modifying enzyme is expressed in a cell of the organism that ultimately gives rise to a germline, thus inducing one or more sequence modifications in t the essential gene, rendering it partially or wholly non-functional, and resulting in death or loss of ability to participate in fertilization if the cell lacks the rescue transgene. In cells comprising the one or more vectors, the partially or wholly non-functional gamete is rescued by the rescue transgene. Progeny carrying the altered gamete containing one or more vectors may be introduced in an environment wherein an increase in a frequency of the vector-bearing progeny is desired relative to a frequency of a wild organism in a population of the organism. In some embodiments, if the vector is linked to a male determining locus, only male, but not female, progeny containing the vector are produced, resulting in an increase in the frequency of males versus females, generation by generation. For example, in some embodiments plant cells comprising the one or more vectors linked to a male sex determining locus give rise to pollen that, because it carries the male-determining gene(s), only gives rise to male, but not female, progeny. Ultimately sufficient females are eliminated that the population collapses. In some embodiments, if the vector is linked to a female determining locus, female vector-bearing adults give rise to ovules that, because they carry the female-determining gene(s), only gives rise to female, but not male, progeny, wherein this results in an increase in the frequency of females versus males generation by generation until sufficient males are eliminated and the population collapses.

In some embodiments, the sex-determining locus is located on the Y chromosome. In some embodiments, the sex-determining locus is located on an autosome. In some embodiments, the sex-determining locus is located on a W chromosome. In some embodiments, the organism gives rise to a haploid gamete. In some embodiments, the diploid adult undergoes meiosis to form haploid gametes before undergoing fertilization to form a new diploid. In some embodiments, the progeny is formed by mating the haploid gametes with an organism of the same species. In some embodiments, the organism is a polyploid that gives rise to gametes with half the adult number of each chromosome type. In some embodiments, the organism is a plant.

In some embodiments, the DNA sequence modifying enzyme is a nuclease or a base editor or a transposase. In some embodiments, the nuclease cleaves and generates one or more double strand breaks in the endogenous copy of the essential gene. In some embodiments, the one or more double strand breaks are repaired to create an altered sequence of the essential gene. In some embodiments, the base editor creates one or more base changes in the endogenous copy of the essential gene to create an altered sequence of the essential gene.

In some embodiments, the one or more base changes comprise one or more point mutations in the endogenous copy of the essential gene. In some embodiments, a transposase mediates integration of vector sequences into a target site. In some embodiments, the rescue transgene is a recoded copy of the essential gene, encoding a protein that is functionally equivalent to a protein encoded by the essential gene, and wherein the DNA sequence modifying enzyme does not modify the rescue transgene.

In some embodiments, the chromosome is an autosome, X chromosome, Y chromosome, Z chromosome, W chromosome or supernumerary chromosome. In some embodiments, the chromosome is an X chromosome. In some embodiments, the chromosome is a Y chromosome. In some embodiments, the chromosome is a Z chromosome. In some embodiments, the chromosome is a W chromosome. In some embodiments, the chromosome is a supernumerary chromosome. In some embodiments, the extra-chromosomal element is a plasmid or a virus.

In some embodiments, the one or more regulatory sequences comprise one or more foreign gene sequences, or one or more alleles of an endogenous chromosomal or extra-chromosomal gene to which the vector has been linked through nearby insertion on the chromosome or extra-chromosomal element that carries the endogenous gene of interest.

In some embodiments, the DNA sequence modifying enzyme is selected from the group consisting of Cas9, Cas-9-related RNA-guided nucleases, ZFN, TALEN, homing endonuclease, natural site-specific nucleases, engineered site-specific nucleases, prime editing enzymes, transposases, base editing enzymes, cytidine deaminase, and adenine deaminase. In some embodiments, the Cas9 is modified. In some embodiments, the modified Cas9 possesses increased expression and catalytic activity relative to an unmodified Cas9. In some embodiments, the modified Cas9 carries one or more introns and/or carries at least one mutation. In some embodiments, the mutation is K918N.

In some embodiments, the vector(s) further comprises one or more additional sequences, wherein the one or more additional sequences allow the one or more vectors to be positioned in a chromosome or an extra-chromosomal element. In some embodiments, the one or more additional sequences is selected from the group consisting of transposase binding site, resistance markers, transgenesis markers, LTRs, recombinase binding site, a sequence with homology to a desired location on the chromosome or the extra-chromosomal element. In some embodiments, the first promoter is selected from the group consisting of a germline promoter, a male specific germline promoter, a female specific germline promoter, a cell-type specific promoter, a tissue-specific promoter, a ubiquitous promoter, a promoter activated at a specific stage of mitosis, and a promoter activated at a specific stage of meiosis. In some embodiments, the first enhancer or promoter is selected from the group consisting of ubiquitin-10, apetala1, agamous, DMC-1, clavata-3 and CaMV35S. In some embodiments, the second promoter is independent of the first promoter. In some embodiments, the second promoter is a U6 promoter.

In some embodiments, a double strand break is repaired by a mechanism selected from the group consisting of non-homologous end joining, microhomology-mediated end joining, and incomplete homologous recombination. In some embodiments, the size of the one or more regulatory sequences ranges from about 0.5 kb to about 500 kb. In some embodiments, the nuclease comprises at least one nuclease domain and one or more DNA binding domains. In some embodiments, the DNA binding domain is a TALE or a zinc finger, the nuclease domain is provided as a single active nuclease domain, such as single chain variants of Fok1.

In some embodiments, a plant, a plant part or a plant seed comprising one or more vectors is provided wherein the one or more vectors comprise: a first gene encoding a rescue transgene; a second gene encoding a DNA sequence modifying enzyme; a first enhancer/promoter operably linked to the second gene encoding the DNA sequence modifying enzyme and optionally, one or more regulatory sequences; a nucleic acid encoding one or more guide RNAs (gRNAs) targeting the DNA modifying enzyme to an endogenous copy of an essential gene, and a second promoter operably linked to the nucleic acid encoding one or more guide RNAs (gRNAs), and optionally one or more cargo sequences. In some embodiments, one or more vectors is positioned in a chromosome or an extra-chromosomal element.

In some embodiments, the vector renders an organism vulnerable to toxicity in the presence of a chemical, compound or condition. In some embodiments, the chemical or compound is an herbicide, pesticide or environmental condition such as temperature, salinity, light or water levels. In some embodiments, the vector renders an organism less harmful to other species in the environment. In some embodiments, gene spread renders the population of an organism resistant to toxicity in the presence of a chemical, compound or environmental condition. In some embodiments, the gene spread results in suppression or elimination of a population. In some embodiments, the vector is spread in a population of plants, where the plant is diploid or polyploid.

In some embodiments, methods for population modification or suppression in a plant is provided. In some embodiments, the methods comprise obtaining the plant and introducing into the plant during the plant's diploid stage, one or more vectors comprising a nucleic acid sequence encoding: a first gene encoding a rescue transgene; a first gene encoding a rescue transgene; a first enhancer/promoter operably linked to a second gene encoding a DNA sequence modifying enzyme and optionally, one or more regulatory sequences. In some embodiments, the DNA modifying enzyme modifies an endogenous copy of an essential gene. The vector may comprise a second promoter operably linked to one or more guide RNAs (gRNAs) targeting the endogenous copy of an essential gene. In some embodiments the vector may comprise one or more cargo genes, encoding a cargo protein to be introduced into the population. The DNA sequence modifying enzyme in a cell or cells of the plant before meiosis that ultimately gives rise to a germline such that the essential gene is rendered partially or wholly non-functional. In some embodiments, the methods comprise generating haploid gametes. In some embodiments, haploid gametes containing one or more vectors in which the essential gene has been rendered partially or wholly non-functional is rescued by the rescue transgene in the vector. In some embodiments, haploid gametes that fail to inherit the rescue transgene dies.

### BRIEF DESCRIPTION OF THE DRAWINGS

FIG. 1A-FIG. 1D show an embodiment of a ClvR construct design and ClvR behavior in a diploid plant

FIG. 1A shows a schematic of the genetic makeup of the ClvR drive element.

FIG. 1B shows an embodiment of ClvR behavior during the diploid parental stage to create loss of function (LOF) alleles schematic of the genetic makeup of the ClvR drive element.

FIG. 1C shows an embodiment of cross of a heterozygous ClvR-bearing female with WT (+/+) pollen.

FIG. 1D shows an embodiment of cross in which pollen from a heterozygous ClvR-bearing male fertilizes ovules of a WT (+/+) female and cross of a heterozygous ClvR female to a heterozygous ClvR male.

FIG. 2A-FIG. 2E show an embodiment of genetic evidence for ClvR-based gamete killing and rescue.

FIG. 2A shows a schematic of crosses used to establish independent ClvR insertions in Arabidopsis plants.

FIG. 2B shows a schematic of test for gametic drive through the male and female germline.

FIG. 2C shows a schematic of frequency of ClvR transmission through the female (left) and male (right) germline.

FIG. 2D shows a schematic of T3 heterozygous crosses for ClvR female (left) and ClvR male (right) utilizing APETALA1 regulatory sequences.

FIG. 2E shows a schematic of T3 heterozygous crosses for ClvR female (left) and ClvR male (right) using the CaMV35S promoter.

FIG. 3A-FIG. 3F show an embodiment of stability of ClvR-based gamete killing and rescue over multiple generations.

FIG. 3A shows embodiments of individual of heterozygous ClvRubq, homozygous ClvRubq and WT plants

FIG. 3B shows embodiments of whole pots of heterozygous ClvRubq, homozygous ClvRubq and WT plants.

FIG. 3C shows embodiments of T3 cross between ClvRubq/+ females and WT was used to generate T4 ClvRubq/+ heterozygous plants.

FIG. 3D shows embodiments of T3 cross between ClvRubq/+ males and WT was used to generate T4 ClvRubq/+ heterozygous plants.

FIG. 3E shows embodiments of frequency of ClvRubq inheritance in crosses in which the T3 grandparent was a ClvRubq/+ heterozygote female and T4 parents were either a ClvRubq/+ female (left six columns) or male (right six columns).

FIG. 3F shows embodiments of frequency of ClvRubq inheritance in crosses in which the T3 grandparent was a ClvR^{ub}q/+ heterozygote male and T4 parents were either female (left six columns) or male (right six columns).

FIG. 4A-FIG. 4B show embodiments of characterization of the target locus following exposure to ClvRubq, and genetic behavior of LOF mutations found in female escapers.

FIG. 4A shows a schematic of the genomic region containing the YKT61 gene along with the approximate locations of four gRNA target sites.

FIG. 4B shows a schematic of fraction of total ovules in individual siliques (circles) that developed into seeds in self-crosses from 4 different genotypes.

FIG. 5A-FIG. 5G show an embodiment of predicted behavior of ClvR for population modification and suppression.

FIG. 5A shows a schematic of modeling process used in predicted behavior of ClvR for population modification and suppression.

FIG. 5B shows a schematic of the population modification when ClvR is introduced as homozygous males at a frequency of 10% of the starting population, which is at carrying capacity, 10,000 individuals, where the mating system is monogamous, polyandrous with 5 males each providing ⅕ of the sperm needed to fertilize all ovules of an individual female and 20 males each providing 1/20 of the sperm needed.

FIG. 5C shows a schematic of the population suppression with a transgene inserted into a recessive locus required for female sporophyte fertility when ClvR is introduced as homozygous males at a frequency of 10% of the starting population, which is at carrying capacity, 10,000 individuals, where the mating system is monogamous, polyandrous with 5 males each providing ⅕ of the sperm needed to fertilize all ovules of an individual female and 20 males each providing 1/20 of the sperm needed.

FIG. 5D shows a schematic of the population suppression with a the ClvR inserted into a locus required for male sporophyte fertility when ClvR is introduced as homozygous males at a frequency of 10% of the starting population, which is at carrying capacity, 10,000 individuals, where the mating system is monogamous, polyandrous with 5 males each providing ⅕ of the sperm needed to fertilize all ovules of an individual female and 20 males each providing 1/20 of the sperm needed.

FIG. 5E shows a schematic of the population modification when ClvR is introduced as homozygous males at a frequency of 20% of the starting population, which is at carrying capacity, 10,000 individuals, where the mating system is monogamous, polyandrous with 5 males each providing ⅕ of the sperm needed to fertilize all ovules of an individual female and 20 males each providing 1/20 of the sperm needed.

FIG. 5F shows a schematic of the population suppression with a transgene inserted into a recessive locus required for female sporophyte fertility when ClvR is introduced as homozygous males at a frequency of 20% of the starting population, which is at carrying capacity, 10,000 individuals, where the mating system is monogamous, polyandrous with 5 males each providing ⅕ of the sperm needed to fertilize all ovules of an individual female and 20 males each providing 1/20 of the sperm needed.

FIG. 5G shows a schematic of the population suppression with a the ClvR inserted into a locus required for male sporophyte fertility when ClvR is introduced as homozygous males at a frequency of 20% of the starting population, which is at carrying capacity, 10,000 individuals, where the mating system is monogamous, polyandrous with 5 males each providing ⅕ of the sperm needed to fertilize all ovules of an individual female and 20 males each providing 1/20 of the sperm needed.

### DETAILED DESCRIPTION

Gene drive occurs when genetic elements-genes, gene complexes, large chromosomal regions or entire chromosomes—are transmitted to viable, fertile progeny at rates greater than those of competing allelic variants or other parts of the genome. There has long been interest in the idea that genetic manipulation of wild populations via gene drive could be used for beneficial purposes. Transgenes or alleles of endogenous loci can be linked with a drive element. The results of modeling and lab experiments demonstrate this can result in spread of these “cargo” genes to high frequency in an extant population. Alternatively, drive can result in population suppression or elimination if spread of the element drives the population towards an unfit set of genotypes (e.g., all male, all females sterile) (Hay et al. 2021; Bier 2022; Raban et al. 2023).

A variety of selfish genetic elements have been considered for bringing about gene drive. These include transposons and homing endonucleases, which spread through over-replication (copying); multigene complexes that bring about female meiotic drive or sperm post-meiotic segregation distortion; and toxin-antidote combinations that cause the death of those (cells, spores, gametes, or progeny) who fail to inherit them from a carrier. Toxin-antidote gene drive elements (TA elements) are particularly interesting as they are found throughout all domains of life: prokaryotes, fungi, animals and plants; and the wide distribution of some of these elements in nature shows they can spread and persist in complex natural environments (Burga et al. 2020; De Carvalho et al. 2022; Wang et al. 2023).

A TA element sits at a fixed chromosomal position and consists of one or more genes that encode linked toxin and antidote functions. The toxin, typically a protein, has the potential to kill or impair the development of those in which it is present, while the antidote, a protein or RNA, suppresses the toxin's activity or expression (Burga et al. 2020; Jurenas et al. 2020). The toxin is trans-acting and is distributed to all meiotic products or progeny of a TA-bearing parent. However, only those that inherit the TA cassette express the antidote, which counteracts the toxin in cis. In consequence, TA elements ensure their presence in the next generation by causing the death of those who fail to inherit them (post-segregational killing), a form of genetic addiction. The death of those lacking the TA cassette results in a relative increase in frequency of those carrying it. Modeling shows that TA elements in sexually reproducing eukaryotes can (depending on the fitness costs associated with carriage of the element and introduction frequency) spread to high frequency even if they do not confer any advantage to their hosts (Hamilton 1967; Wade and Beeman 1994; Hastings 1994; Smith 1998; Ward et al. 2011a; Marshall and Hay 2012; Oberhofer et al. 2019; Champer et al. 2020b, 2020a; Martinossi-Allibert et al. 2021; Li and Champer 2023).

TA elements that exist in nature evolved in specific genomic, organismal and ecological contexts, and the mechanisms of action, associated gene regulation and species-specific information on development (timing and levels of gene and protein expression and localization) do not transfer to bring about drive in other species. Similar considerations apply to synthetic Medea TA elements engineered in Drosophila in which the toxin is an engineered transient loss-of-function LOF) of a maternally expressed gene whose product is essential for embryogenesis and the antidote is a zygotically expressed transgene that restores this missing function in a just-in-time fashion (Chen et al. 2007; Akbari rt al. 2014; Buchman et al. 2018).

Recently, in an effort to create a chromosomal TA-based gene drive system utilizing a LOF toxin that consists of a simple and extensible set of components that can plausibly be implemented across diverse species Cleave and Rescue (ClvR) element (Oberhofer et al. 2019; Oberhofer et al. 2020; Oberhofer et al. 2021; Oberhofer et al. 2021), also referred to as Toxin Antidote Recessive Embryo (TARE) was developed in related implementations (hereafter referred to as ClvR, a name that captures the key mechanisms involved) (Champer et al. 2020; Metzloff et al. 2022). A ClvR element encodes two activities. The first component, the Cleaver/Toxin, is a DNA sequence-modifying enzyme such as Cas9 and multiple guide RNAs (gRNAs). These are expressed in the germline or cells that will become the germline, though germline-specific expression is not required. In the case of Cas9, it disrupts-through cycles of cleavage and end joining that continue until the target site is destroyed-endogenous versions of a haplosufficient (and in some contexts haploinsufficient or haplolethal) essential gene, wherever it is located. Inaccurate repair at multiple positions in the coding region of the essential gene creates loss-of-function (LOF) alleles. These are the potential toxin. The second component of ClvR, the Rescue/Antidote, is simply a recoded version of the essential gene resistant to cleavage and gene conversion with the cleaved version, expressed under the control of regulatory sequences sufficient to rescue the LOF phenotype. The Rescue functions to guarantee the survival of those who carry ClvR. LOF alleles of the essential gene, which segregate and exist independently of ClvR, perform their toxin function when they find themselves (potentially many generations later) in homozygotes that die because they lack the ClvR-derived source of essential gene function. In contrast, those who inherit ClvR and its associated Rescue survive. In this way, as with other TA-based selfish genetic elements, ClvR spreads by causing the death of those that lack it. This results in cells, organisms, and ultimately populations becoming dependent on (addicted to) the ClvR-encoded Rescue transgene for their survival.

In Drosophila, autonomous ClvR/TARE elements have been shown to spread in WT populations to transgene fixation (all individuals carry at least one copy) (Oberhofer et al. 2019; Champer et al. 2020; Oberhofer et al. 2020; Oberhofer et al. 2021; Metzloff et al. 2022). Other features, such as the ability to bring about strong, but self-limited drive, engage in multiple cycles of population modification that replace old content with new, and bring about population suppression using a conditional Rescue have also been demonstrated (Oberhofer et al. 2020; Oberhofer et al. 2021). Multiple other configurations of the components that make up ClvR/TARE have been proposed, and modeling predicts they can bring about drive in animals with a diversity of interesting characteristics for population modification or suppression (Champer et al. 2020; Champer et al. 2021).

While ClvR-type drive in animals is has been implemented through the killing and rescue of specific zygote genotypes, TA drive based on killing and rescue of gametes has never been demonstrated. There are several problems. First, in females the gamete is chosen through differential segregation of one of the products of meiosis within the common cytoplasm of the oocyte and thus there is no opportunity for the antidote to select for carriers. Second, in spermatogenesis the haploid spermatid products of a meiosis are connected by cytoplasmic bridges until late in development, and active content sharing of many but not all products (e.g. Bhutani et al. 2021) limits opportunities for bringing about differential killing and survival (Immler et al. 2019). Thus, in animals, haploid gametes lacking ClvR and a functional copy of the essential gene will generally survive and be functional because essential gene products utilized during the haploid stage are expressed during the diploid stage and shared between the products of meiosis (Braun et al. 1989; Hime et al. 1996; Niedenberger et al. 2018). In other organisms like plants in which extensive transcription occurs during the haploid stage, it is possible that gametes could be lost if transcription of the targeted essential gene is required during the haploid phase for gamete survival or function.

In plants, meiotic products undergo additional rounds of mitosis, developing into multicellular haploid gametophytes (the female megagametophyte and male microgametophyte) that produce egg or sperm and other essential support cells. This requires extensive expression of the haploid genome (Somers et al. 2023). These features of plant gamete development are reflected in the many recessive mutants (no somatic phenotype in the heterozygous diploid or polyploid parent; the sporophyte stage) that cannot be transmitted through one or the other sex, often identified through sex-specific transmission ratio distortion (e.g. Howden et al. 1998; Christensen et al. 1998). These characteristics suggest that organisms in which extensive transcription occurs during the haploid stage (e.g., plants, fungi), could support a gene drive based on a Cleave and Rescue mechanism that causes the death of gametes that fail to inherit ClvR from a parent, a hypothesis not previously explored or tested.

Described herein is a novel mechanism for population modification or population suppression in diploid or polyploid organisms, particularly in diploid or polyploid plants. In some embodiments, the present disclosure is related to vectors and methods for DNA sequence modification-based modification of populations, particularly in plant populations and beneficial and commercial applications of the vectors and methods. In some embodiments, the methods for DNA sequence modification-based modification of populations are mediated by targeting a gene whose expression during haploid stage is required for the survival and development of gametes. In some embodiments, the present disclosure relates to methods for performing spread of a gene or multi-gene cassette in a diploid or polyploid organism. In some embodiments, the gene spread mediates population modification of the diploid or polyploid organism. In some embodiments, the gene spread mediates population suppression of the diploid or polyploid organism. In some embodiments, the method disclosed herein is mediated by modifying to a loss-of-function state (point mutation, indel, large deletion or insertion) an endogenous gene that is essential for survival or fertilization ability of the gametes of the organism.

In some embodiments, the cleavage and creation of loss-of-function (LOF) alleles in the target essential gene occurs before meiosis. In this way both copies of the target gene are rendered non-functional before the first meiotic division segregates the vector-bearing chromosome into one gamete (which survives) and the non-vector-bearing gamete into the other one, which dies. Also provided herein are cells comprising one or more vectors disclosed herein. In some embodiments, plants, plant parts or plant seeds comprising one or more vectors are provided.

### Definitions

As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein.

In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive. Unless defined otherwise, technical and scientific terms used herein have the same meaning as commonly understood by one of ordinary skill in the art to which this invention belongs. See, for example Singleton et al., Dictionary of Microbiology and Molecular Biology 2nd ed., J. Wiley & Sons (New York, N. Y. 1994); Sambrook et al., Molecular Cloning, A Laboratory Manual, Cold Springs Harbor Press (Cold Springs Harbor, N.Y. 1989). For purposes of the present invention, the following terms are defined below. It is to be understood that both the foregoing general description and the following detailed description are exemplary and explanatory only and are not restrictive of the invention as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting.

As used in this specification and claims, the singular forms “a,” “an” and “the” include plural references unless the content clearly dictates otherwise.

As used herein, “about” means a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

As used herein, “regulatory element” refers to nucleic acid elements that can influence the expression of a coding sequence (for example, a gene) in a particular host organism. These terms are used broadly and encompass all elements that promote or regulate transcription, including promoters, core elements required for basic interaction of RNA polymerase and transcription factors, upstream elements, enhancers, and response elements (see, for example, Lewin, “Genes V” (Oxford University Press, Oxford) pages 847-873).

As used herein, a promoter is a recognition site on a DNA sequence or group of DNA sequences that provides an expression control element for a structural gene and to which RNA polymerase specifically binds and initiates RNA synthesis (transcription) of that gene.

As used herein, the term “insertion site” refers a nucleic acid sequence that allows for insertion of the constructs as provided herein into a genome of a multicellular organism (for example, an insect genome). In some embodiments, a construct as provided herein can comprise a “insertion sequence” that allows for insertion of the construct into a genome of the host organism. Some embodiments that can be employed include the piggybac transposable element, mariner type transposable elements, and the P-element. Also, plasmids can be site specifically integrated into the genome using attb/attp or even by using CRISPR/Cas9, TALEN, MegaTAL and homologous recombination.

As used herein, a “vector,” interchangeably referred to as a transgenic construct, a targeting construct, or simply a construct, is a nucleic acid. As used herein, “nucleic acid” refers to deoxyribonucleic acid (DNA). In some embodiments, nucleic acid may refer to ribonucleic acid (RNA). In some embodiments, the construct as provided herein comprise one or more regulatory elements. Exemplary regulatory elements in prokaryotes include promoters, operators and ribosome binding sites. Regulatory elements that are used in eukaryotic cells can include, without limitation, transcriptional and translational control sequences, such as promoters, terminators, enhancers, insulators, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell. For example, a promoter is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions (for example, a change in temperature).

As used herein, “homologous recombination” refers to exchange of nucleotide sequences between two identical nucleic acid sequences. Homologous recombination also refers to exchange of nucleotide sequences between two similar nucleic acid sequences. In some embodiments, when the two nucleic acid sequences are similar, a similarity between the two nucleic acid sequences can be about 90% to about 99.9%. In some embodiments, the similarity between the two nucleic acid sequences can be about 90, 91, 92, 93, 94, 95, 96, 97, 98, 99, 99.1, 99.2, 99.3, 99.4, 99.5, 99.6, 99.7, 99.8 or 99.9%.

As used herein, the term “gene” as used herein have their plain and ordinary meaning as understood in light of the specification, and generally refers to a portion of a nucleic acid that encodes a protein or functional RNA; however, the term may optionally encompass regulatory sequences. It will be appreciated by those of ordinary skill in the art that the term “gene” may include gene regulatory sequences (e.g., promoters, enhancers, etc.) and/or intron sequences. It will further be appreciated that definitions of gene include references to nucleic acids that do not encode proteins but rather encode functional RNA molecules such as tRNAs and miRNAs. In some cases, the gene includes regulatory sequences involved in transcription, or message production or composition. In other embodiments, the gene comprises transcribed sequences that encode for a protein, polypeptide or peptide. In keeping with the terminology described herein, an “isolated gene” may comprise transcribed nucleic acid(s), regulatory sequences, coding sequences, or the like, isolated substantially away from other such sequences, such as other naturally occurring genes, regulatory sequences, polypeptide or peptide encoding sequences, etc. In this respect, the term “gene” is used for simplicity to refer to a nucleic acid comprising a nucleotide sequence that is transcribed, and the complement thereof. As will be understood by those in the art, this functional term “gene” includes both genomic sequences, RNA or cDNA sequences, or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like.

As used herein, an “essential gene” is defined as a gene that is critical for survival, growth or fertility, and whose loss of function is either lethal, prevents growth or is sterilizing. Some essential genes are critical for survival under all circumstances. Some essential genes are critical for survival only under particular circumstances and/or particular environmental conditions (e.g., in the presence of toxic drugs, toxins, etc., or in the absence of nutrients, vitamins, etc.). In some embodiments, more than one or more endogenous copy of the essential gene are present. In some embodiments, when one or more endogenous copies of the essential gene are present, they are alleles or allelic variants of the essential gene. As used herein, the “endogenous copy” refers to the wild-type, functional versions of the essential gene.

As used herein, modifying a gene may be carried out with techniques that employ targeted mutagenesis. These techniques include, but are not limited to, those that utilize endonucleases to generate single-strand and double-strand DNA breaks that activate DNA repair pathways. Genome editing techniques may also comprise systems that enable targeted editing at any genomic locus. These targeting systems include, but are not limited to, polypeptides, such as, Transcription Activator-Like Effectors (TALEs) and zinc fingers (ZFs), or nucleic acids, such as, Clustered Regularly Interspaced Short Palindromic Repeats/Cas (CRISPR/CAS) single guide RNAs or NgAgo (Argonaute) single strand DNAs.

As used herein, gene modification encompasses any insertion, deletion, or substitution of a nucleotide sequence of any size and nucleotide content, any epigenetic modification to any number of nucleotides, or a combination thereof. A gene modification, as used herein, may also encompass introduction of one or more exogenous coding nucleic acids that do not integrate into the unmodified genome, yet are capable of autonomous replication. In certain embodiments, a modification to an endogenous gene or regulatory element thereof may be a deletion, a substitution, or an insertion that reduces expression of the endogenous gene or the polypeptide for which it encodes. In some specific embodiments, the modification may be an indel, wherein the indel may cause a frameshift mutation, a missense mutation, a nonsense mutation, a neutral mutation, or a silent mutation. In specific embodiments, a modification to a regulatory element of an endogenous gene may alter or eliminate a function of the regulatory element. In further contemplated embodiments, the modification may comprise a nucleic acid sequence that provides exogenous control of endogenous gene, mRNA, or polypeptide expression levels. In specific embodiments, the modification may also disrupt a post-translational process of a polypeptide encoded by an endogenous gene. Post-translational processes in certain embodiments may be post-translational modifications, protein sorting, or proteasomal degradation.

As used herein, a gene modification technique is a technique that is known to those in the art that can modify the genome of a cell including, but not limited to, genome editing, site-specific genetic recombination, epigenetic modifications, and genetic transformation.

As used herein, transformation is a process of introducing a DNA sequence or construct (e.g., a vector or expression cassette) into a cell or protoplast in which that exogenous DNA is incorporated into a chromosome or is capable of autonomous replication.

As used herein, the term “plant” refers to a diploid, haploid, tetraploid or polyploid plant. As used herein, the term “plant part” refer to cells, tissues, organs, seeds, and severed parts (e.g., roots, leaves, and flowers) that retain the distinguishing characteristics of the parent plant. “Seed” refers to any plant structure that is formed by continued differentiation of the ovule of the plant, following its normal maturation point at flower opening, irrespective of whether it is formed in the presence or absence of fertilization and irrespective of whether or not the seed structure is fertile or infertile. A plant part may be any part of the plant from which another plant may arise.

As used herein, a genetically modified plant is a plant comprising at least one genetically modified cell. A genetically modified plant may be regenerated from a genetically modified cell or plant part comprising genetically modified cells, and thus the genetic modification may be heritable and inherited by progeny thereof. The progeny thereof that inherit the genetic modification are also considered genetically modified plants. A genetically modified plant, as used herein, also refers to a plant in which at least one genetically modified cell is introduced to a plant or arises as a result of genetic modification techniques directly applied to the plant.

As used herein, “gene drive” refers to a situation in which genetic elements—including alleles of specific genes, gene complexes, entire chromosomes or endosymbiotic bacteria—are transmitted to viable, fertile progeny at rates greater than those due to Mendelian transmission, resulting in an increase in their frequency in the population over time, even if their presence results in a fitness cost to carriers. Without being limited by any particular theory, gene drive can occur by a number of mechanisms. In some embodiments, the gene drive is engineered. In some embodiments, the gene drive is engineered depending on the context and environment in which it occurs.

In some embodiments, “population suppression” refers to a term or a method that reduces or eliminates a population of organisms, a certain sex or a subspecies of organisms, by introducing an engineered gene-drive that spreads through the population causing reduced fertility, viability or decline in population of a certain population, sex or a subspecies of the organism.

In some embodiments, “population modification” refers to a term that alters one or more traits of a population of an organism. In some embodiments, the modified population may exhibit one or more of a change in its habitability, fitness, ability to provide ecosystems services, resistance or sensitivity to pests, weeds, invasive species, chemicals, compounds or environmental conditions like drought, flooding, salinity, etc. In some embodiments, modification of a population can include modulation of gene expression (e.g., gene activation or gene expression).

As used herein, “haploid” refers to the presence of a single set of chromosomes in an organism's cells and “diploid” refers to the presence of two complete sets of chromosomes in an organism's cells, with each set of chromosomes being contributed by each parent.

As used herein, “polyploid” refers to the presence of some number greater than two as the number of each chromosome type in the adult, sporophytic phase of the plant life cycle. Gametes of a polyploid contain half the adult number of chromosomes of each type found in the adult.

In some embodiments, “pest” is a living organism that is invasive, destructive or detrimental to humans, human concerns, livestock, agriculture or ecosystems. The term pest may include plant, weed, parasite, vermin or pathogen. The term weed as used herein may refer to a plant that is undesirable in a particular situation or invasive outside its native habitat. As used herein, the term “invasive species” may refer to a plant non-native (or alien) to the ecosystem under consideration whose introduction causes or is likely to cause economic or environmental harm or harm to human, plant or animal health.

Overview of CleaveR-Based Gene Drive that Acts by Killing Progeny Who Fail to Inherit from a Parent

CleaveR [Cleave and Rescue (ClvR)] refers to a synthetic genetic element that can spread a linked gene to high frequency in a population of an organism. ClvR is composed of two components. The first, germline-expressed DNA sequence modifying enzyme, such as Cas9 and guide RNAs (gRNAs), cleave and disrupt versions of an essential gene located elsewhere in the genome. The second is a rescue gene that is a version of the essential gene that is resistant to cleavage and provides essential gene function. ClvR spreads by creating conditions in which progeny lacking ClvR die because they have no functional copies of the essential gene. In contrast, those progeny who inherit ClvR survive, resulting in an increase in ClvR frequency in a population. The operative principle in all cases is that in the relevant cell type, or in a multicellular organism, in some fraction of these cells, all or most copies of the endogenous copies of the essential gene are altered so as to produce non-functional copies of the gene. This results in death of all cells that fail to inherit one or more copies of the rescue transgene. The DNA sequence modifying enzyme and the rescue transgene are tightly linked and behave as a single genetic unit.

In some embodiments, a Cleave and Rescue (ClvR) system for bringing about gene drive-mediated population modification or suppression in organisms such as plants, is provided. In some embodiments the organisms are diploid or polyploid. In some embodiments, the ClvR system comprises a site-specific DNA-modifying enzyme (e.g., Cas9) and the gRNAs that guide it to specific targets within the genome of the organism, sequences sufficient to direct gene expression in cells that will become the germline, an essential gene that is the target of the gRNAs and the DNA-modifying enzyme, and a recoded version of the essential gene resistant to sequence modification and that is able to rescue the loss of function mediated by sequence modification of the essential gene. In some embodiments, the cells are not germline cells.

In some embodiments, the ClvR system mediated gene-drive is used to drive modification of a population of an organism. In some embodiments, the gene-drive mediated population modification using the ClvR system comprises inserting into cells (e.g., germline cells) of an organism of the population to be modified, for example a plant, one or more vectors that increase the fitness of the organism. In some embodiments, the method includes inserting into the cells (e.g., germline cells) of an organism, for example a plant, one or more vectors that renders the organism resistant to an external stimulus. In some embodiments, the one or more vectors carrying one or more cargo genes are inserted into the plant, wherein the expression of one or more cargo genes in the plant renders the plant resistant to an external stimulus. In some embodiments, the external stimulus is a condition. Some non-limiting examples of the condition include environmental conditions such as temperature, stress, drought, inadequate soil fertility, nutrient deficiencies, pollution, salinity, light, water levels, presence of a weed or pest, competitor or pathogen. In some embodiments, the condition is a herbicide or another chemical or a composition that is applied to the plant. In some embodiments, the modification renders the organism drought resistant. In some embodiments the modification renders the plant resistant to a pest or disease.

In some embodiments, the components of the ClvR system for mediating modification of a population can be located near the target essential gene. In some embodiments, the components of the ClvR system for mediating modification of a population can be located distant from the target essential gene.

In some embodiments, the ClvR system mediated gene-drive is used for population suppression of an organism. In some embodiments, the ClvR system mediated gene-drive for population suppression is mediated by inserting the elements of the ClvR system into a gene required for diploid fertility of one sex, thereby disrupting the gene required for diploid fertility of one sex. In this configuration, heterozygotes of both sexes for the elements of the vector are fertile, while homozygotes for one sex are sterile. As the vector spreads, heterozygotes end up mating with each other. Since only vector-bearing gametes can survive, the only survivors are homozygous for the vector. Homozygotes for one of the sexes are sterile. Homozygotes for the other are fertile and keep spreading the vector. Eventually this results in a population crash when fertile members of one sex are no longer present.

In some embodiments, the ClvR system mediated gene-drive for population suppression is mediated by inserting the elements of the ClvR system next to or within a locus whose presence is sufficient for adult male or female sex determination. In some embodiments, the presence of the elements of the vector near a male-determining locus means that all surviving gametes produced by a male will carry the male-determining locus (the Y chromosome) and the vector. These males will produce more males and so on, never producing female progeny. Eventually the population runs out of females and goes extinct.

In some embodiments, a non-gene drive mediated population suppression using the ClvR system is provided. In some embodiments, an autosomal ClvR (ClvR^{FS}) in which males produce fertile ClvR-bearing males (which produce only ClvR-bearing pollen) and sterile females. In some embodiments, periodic releases of males with these features can bring about population suppression or elimination by driving a progressive decrease in the number of fertile females. In some embodiments, ClvR^{FS} is generated by placing the components of ClvR system within (thereby disrupting) a gene whose expression in female gametes is required for their fertility. In plants, males carrying this construct produce ClvR-bearing pollen. In contrast, daughters that inherit ClvR are sterile. The female gametes that inherit the ClvR die because they lack the female gamete essential gene while those that lack ClvR die because they lack a functional copy of the gamete essential gene that was targeted by ClvR. In some embodiments, the components of ClvR system can be placed in a neutral position with a transgene that is expressed and blocks female fertility so as to get same effect.

When the CleaveR construct is present in an organism, wildtype copies of the essential gene are targeted for cleavage and inactivation. The individuals carrying ClvR themselves do not experience any cost from this cleavage, which happens in the germline and also in some cases in somatic cells, because they also carry a tightly linked copy of the rescue transgene. However, the gametes they pass on will in many cases not carry a functional copy of the endogenous essential gene, and they may also lack the ClvR construct. As a result, the gametes that lack the ClvR construct will die while the gametes with the ClvR construct will survive. In some cases, the Cas9/gRNA complexes will also be deposited into oocytes/eggs, resulting in cleavage of the endogenous copy of the essential gene in early embryos that do not carry the ClvR construct. In all of these cases, which arise through normal Mendelian segregation of chromosomes during meiosis in males and females, and in some cases diffusion or transport of Cas9/gRNA into daughter cells or products of cell-cell fusion (fertilization), progeny is often created that carry no functional copies of the essential gene. These individuals are of low fitness (dead, sterile or otherwise dysfunctional) and do not contribute further to the population.

The above behavior results in some loss in each generation of chromosomes and individuals that do not carry the CleaveR. This results, over multiple generations, in a progressive increase in the frequency of CleaveR-bearing individuals. Modeling, discussed further below, shows that under a variety of conditions CleaveR is predicted to spread to high frequency such that most or all individuals in the population bear at least one copy of the CleaveR chromosome. The CleaveR chromosome is in some sense “held” in the population because as it has been spread (and the mechanism by which it has been spreading) it has necessarily caused inactivation of most or all of the wildtype copies of the essential gene. Thus, the population has become “locked” into a configuration in which it now depends on (is addicted to) the presence of CleaveR in order to maintain viability or fertility.

A similar principle, cleavage associated with rescue of those who carry the CleaveR vector, could allow CleaveR to act as a gamete or spore killer, or force its inheritance in conditions in which it is episomal (as in a plasmid). In each case the presence of the CleaveR element selects for those who carry it, and against those who fail to inherit it, provided that a activity of the essential gene is needed for the survival/fertility/growth of the cell.

In one implementation of this system, detailed below in the examples and sometimes referred to as CleaveR (also referred to herein as ClvR), the DNA-modifying enzyme is selected from the group consisting of Cas9, Cas-9-related RNA-guided nucleases, ZFN, TALEN, homing endonuclease, natural site-specific nucleases, engineered site-specific nucleases, prime editing enzymes, transposases, base editing enzymes, cytidine deaminase, and adenine deaminase. In some embodiments, while a nuclease that includes a member of the RNA-guided nucleases, such as Cas9 is exemplified, any DNA-modifying enzyme that can be specifically targeted to a gene of interest (here an essential gene) can be used. In this implementation Cas9 is expressed in the germline of male, females, or both sexes. Multiple gRNAs are also expressed, preferably three or four of them. They are designed to engage in Watson-Crick base pairing with, and therefore target for cleavage, distinct sequences within a target essential gene, so as to bring about its cleavage at multiple sites. These multiple breakages are expected to result in the creation of repair products—deletions, base changes, small additions—that create a non-functional version of the targeted essential gene. In summary, the purpose of the nuclease is to bring about loss-of-function mutants of the targeted gene. There are two important characteristics of the system described thus far. First, the cassette encoding the nuclease can sit at any position in the genome. However, as discussed herein, in some embodiments the cassette is placed in a desired position to have a desired effect on the population. Second, the gene being targeted for inactivation is in some sense an essential gene: required for organism survival or fertility, broadly defined as fitness.

In some embodiments, the essential gene is required for the survival of the organism. In some embodiments, the essential gene is required for fertility of the organism. In some embodiments, the essential gene is required for diploid fertility. Some non-limiting examples of essential genes that can be targeted include Bell1, opr3, ms1, ms2, non-dehiscence1, delayed-dehiscence1, POLLENLESS3, rh32, rh29, atrh36, slow walker3. In males these include bcp1, AtMago, PIRLI and PIRL9.

In several embodiments, a site-specific DNA-modifying enzyme is used. In several embodiments, restriction enzymes are used, particularly when double strand breaks are desired at multiple regions. In several embodiments, a bioengineered nuclease is used. In some embodiments, one or more of a Zinc Finger Nuclease (ZFN), transcription-activator like effector nuclease (TALEN), meganuclease and/or clustered regularly interspaced short palindromic repeats (CRISPR/Cas9) system are used to modify the target essential gene loci.

The CleaveR gene drive system also comprises a gene that can rescue the lethality or infertility of those individuals in which both copies (for a diploid) of the essential gene, have been inactivated (or more generally all copies, in the case of a polyploid). In embodiments, the rescue gene is a version of the essential gene. In embodiments where a rescue gene is a version of the essential gene, resistance to cleavage is brought about by recoding the transgene so that it no longer is inactivated by the DNA modifying enzyme. For example, in some embodiments the rescue transgene is a recoded version of the essential gene that does not productively interact with the guide RNA Cas9 complex, according to rules that are well known in the field. Further recoding of the rescue transgene, in both the coding region and non-coding and regulatory regions, is also carried out. This recoding is done so as to minimize homology between the wildtype, endogenous version of the gene and the rescue version of the gene. In some embodiments this recoding may also minimize/eliminate the possibility that the cleaved version of the wildtype endogenous essential gene can be repaired and restored to functionality through ectopic homologous recombination, using the rescue transgene as a template for repair based on existing homology at the broken ends of the former. The literature provides guidance on the level of homology needed to prevent or promote homologous recombination. Without being limited by any particular theory, recoding can successfully achieved even when the rescue transgene has essentially no nucleotide homology to the endogenous copy of the gene. Demonstration that this can be achieved comes from multiple reports showing that bacterial and/or human versions of essential genes can successfully replace their yeast counterparts, resulting in yeast with high fitness. In some embodiments, the rescue gene is of unrelated sequence, wherein the rescue transgene encodes a protein that is functionally equivalent to a protein encoded by the essential gene.

In the CleaveR construct, also often referred to as the vector or the construct, when the DNA sequence modifying enzyme and the rescue transgene are located near each other (tightly linked), they behave as a novel selfish genetic element, able to spread itself into a population and/or maintain itself in a population (bring about population replacement/modification) under a variety of conditions that include varying levels of fitness cost associated with carrying the vector and any associated cargo genes, and introduction frequencies. The details of these vectors are described in PCT/US2018/030990 and U.S. Pat. No. 11,965,172B2, the entirety of which is incorporated by reference herein.

### Single Locus

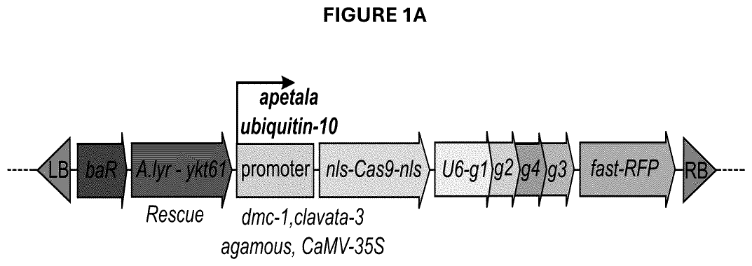

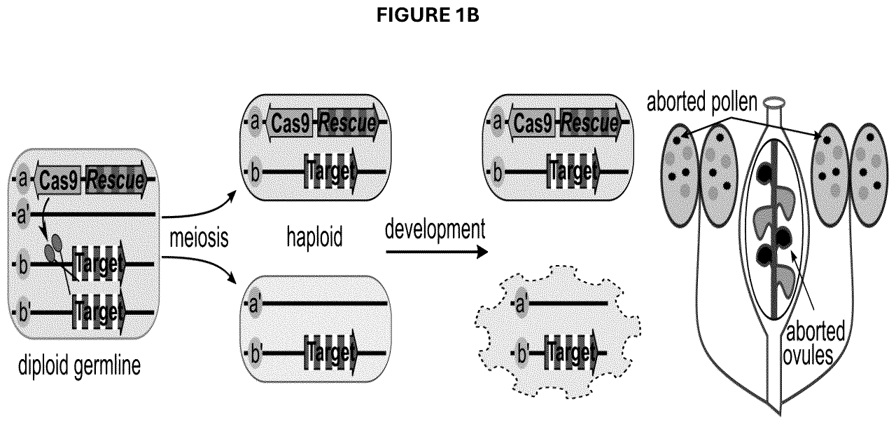

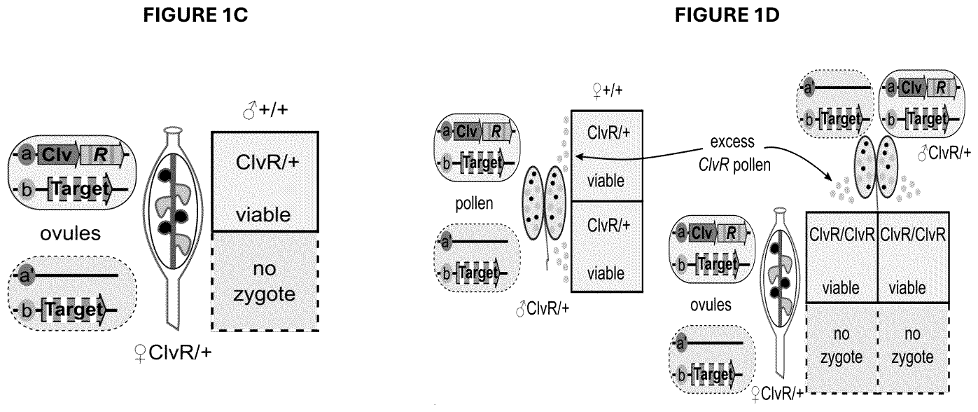

In the single locus CleaveR construct, also often referred to as the vector or the construct, the DNA sequence modifying enzyme and the rescue transgene are located near each other (tightly linked), along with any cargo genes. In short, they move together from generation to generation, as a single gene multigene cassette (see also FIG. 1A). In some embodiments, the vector comprises a DNA modifying enzyme modifies an endogenous copy of an essential gene, a rescue transgene, a nucleic acid encoding one or more guide RNAs (gRNAs) targeting the endogenous copy of an essential gene, regulatory sequences and optionally one or more cargo sequences. They behave, as a novel selfish genetic element, able to spread itself into a population and/or maintain itself in a population (bring about population replacement) under a variety of conditions that include varying levels of fitness cost associated with carrying the vector and any associated cargo genes, and introduction frequencies. The details of these vectors are described in PCT/US2018/030990, the entirety of which is incorporated by reference herein.

In some embodiments, the gene drive disclosed herein is an alternative form of gene drive that utilizes Cas9 or other nucleases to bring about cleavage and repair of an essential gene that does not involve or require homing, though homing can potentially contribute to drive. This form of gene drive can also make use of base editing enzymes such as adenosine or cytosine deaminase to modify specific bases to create non-functional versions of an essential gene. It can also use Search and Replace Prime editing, which uses a Cas9 nickase linked to a reverse transcriptase, and a modified gRNA to introduce base changes or insertions or deletions. Without being limited by any particular theory, the mechanism simply involves a DNA sequence modifying enzyme such as Cas9, a set of gRNAs targeting an essential gene for cleavage, base editing, or Search and Replace Prime editing and a recoded version of the target essential gene that is immune to modification, linked as a single construct (by linked it is meant that they are not separated from each other during meiotic or other forms of recombination). This gene drive method is known as single locus ClvR. In some embodiments, individuals carrying one or more copies of this construct bring about modification of the sequence of one or more copies of the endogenous version of the essential gene such that it is no longer functional. Individuals who end up inheriting only non-functional versions of the essential gene die or are sterile, while those that carry one or more copies of the construct, which includes a rescue transgene, will survive and/or be fertile. Over multiple generations this behavior is predicted to result in the spread of the construct/vector into the population at the expense of the wild types version of the same chromosome.

### Two-Locus

In some embodiments, two locus gene drive as provided in U.S. Pat. No. 11,965,172B2 (the entirety of which is incorporated by reference here), can be applied to any of the single locus embodiments described herein. That is, any of the embodiments provided herein can be modified such that there are effectively two or more loci in the system. In some embodiments, two vectors (“a two-vector system”) are provided for the implementation of the various embodiments provided herein. In a two locus configuration, one or both components required for DNA sequence modification is located at a different position in the genome from the Rescue and/or cargo transgenes. Putting these components at different positions in the genome means that while the components start being linked together, they segregate from each other over time. Importantly, as the DNA sequence modification component segregates from the Rescue, it will eventually be lost from the population in loss-of-function individuals lacking Rescue. As this component disappears, gene drive is lost, thus making the drive self-limiting. Without being limited by any particular theory, the fact that two locus gene drive wanes over generations provides two locus systems (such as ClvR) with three important new, unique features not exhibited by single locus systems. As a shorthand, embodiments are provided herein with respect to CLvR, however, these embodiments can be employed in the other embodiments provided herein as well (as appropriate).

First, gene drive for a given population introduction frequency is limited in time and occurs because the frequency of one or both Cas9/gRNA components decreases over time. Once these alleles are at low frequency or are eliminated, drive can no longer occur. However, even though drive is eliminated, the Rescue/Cargo can remain at genotype fixation. Without being limited by any particular theory, this occurs because all (or nearly all) wildtype alleles of the essential gene have been eliminated, locking the population into a Cargo/Rescue-bearing state.

The second feature of two locus ClvR is that drive is limited in space. It is local rather than global, as with single locus ClvR. This is because as the transgene-bearing organisms distribute in space from a source through migration, the frequency of the Cas9/gRNA components will decrease. Drive of the Cargo/Rescue only occurs when the frequency of Cas9/gRNA (or that of other RNA-guided DNA sequence modifying enzymes such as base editors or Prime editors) is high enough to bring about high frequency creation of LOF alleles of the essential gene. In particular, when two locus ClvR individuals are migrating into a neighboring population composed mostly of wildtypes, the independent segregation of the two chromosomes means that Cas9/gRNA-bearing individuals will often find themselves without a copy of the Rescue, and therefore die. In the absence of levels of LOF allele creation sufficient to create many LOF homozygotes, drive of the Cargo/Rescue into the population will not occur.

The third feature of two locus ClvR is that with it, unlike with single locus ClvR, reversibility to a population that lacks the Cargo/Rescue and the cleaved allele can be achieved by dilution of a transgene-bearing population with wildtype individuals. Whenever the presence of the Cargo/Rescue results in some fitness cost, dilution can lead to elimination of drive, Cargo and LOF alleles of the essential gene, from the population. In contrast, with single locus ClvR, reversibility cannot easily be achieved through dilution because the drive is so powerful.

The above three features are useful to implement in order to have the gene drive mechanisms function within regulatory frameworks. Central to these developments are aspects of confinement and reversibility. Two locus versions of ClvR can limit the spread of transgenes to limited locations in which their presence is sought and the population is restored to the pre-transgenic state in addition to bringing about population alteration to a high frequency of transgene-bearing individuals under a variety of conditions of fitness cost and introduction frequency.

In some embodiments of the two vector system, the first vector comprises a DNA sequence modifying enzyme, wherein the DNA sequence modifying enzyme modifies an endogenous copy of an essential gene, and a promoter is operably linked to the DNA sequence modifying enzyme, and second vector comprising a rescue transgene sequence and a rescue transgene promoter operably linked to the rescue transgene sequence. In some embodiments, the two vectors are positioned on a single chromosome at a distance from each other. In some embodiments, the two vectors are positioned on a single extrachromosomal element at a distance from each other. In some embodiments, the two vectors are positioned on two different chromosomes. In some embodiments, the first vector is positioned on a chromosome and the second vector is positioned on an extrachromosomal element. In some embodiments, the second vector is positioned on a chromosome and the first vector is positioned on an extrachromosomal element. In some embodiments, the second vector optionally comprises one or more cargo sequences.

In some embodiments of the two-vector system, the first vector comprises a first sequence encoding a first component of a DNA sequence modifying complex, and a second sequence encoding the second component of the DNA sequence modifying complex wherein the DNA sequence modifying complex modifies an endogenous copy of an essential gene, and a first promoter that is operably linked to the first sequence encoding the first component and a second promoter is operably linked to the second sequence encoding the second component of the DNA sequence modifying complex, and a second vector comprising a rescue transgene sequence and a rescue transgene promoter operably linked to the rescue transgene sequence. In some embodiments, the two vectors are positioned on a single chromosome at a distance from each other. In some embodiments, the two vectors are positioned on a single extrachromosomal element at a distance from each other. In some embodiments, the two vectors are positioned on two different chromosomes. In some embodiments, the first vector is positioned on a chromosome and the second vector is positioned on an extrachromosomal element. In some embodiments, the second vector is positioned on a chromosome and the first vector is positioned on an extrachromosomal element. In some embodiments, the second vector optionally comprises one or more cargo sequences.

In some embodiments of the two-vector system, the first vector comprises a first sequence encoding a first component of a DNA sequence modifying complex, wherein the DNA sequence modifying complex modifies an endogenous copy of an essential gene, and a first promoter that is operably linked to the first sequence encoding the first component of the DNA sequence modifying complex, a rescue transgene, and rescue transgene promoter operably linked to the rescue transgene, and optionally one or more cargo transgenes, and a second vector comprising a second sequence encoding a second component of the DNA sequence modifying complex, and a second promoter operably linked to the second component of the DNA sequence modifying complex. In some embodiments, the two vectors are positioned on a single chromosome at a distance from each other. In some embodiments, the two vectors are positioned on a single extrachromosomal element at a distance from each other. In some embodiments, the two vectors are positioned on two different chromosomes. In some embodiments, the first vector is positioned on a chromosome and the second vector is positioned on an extrachromosomal element. In some embodiments, the second vector is positioned on a chromosome and the first vector is positioned on an extrachromosomal element. In some embodiments, the first vector optionally comprises one or more cargo sequences.

In some embodiments, a two-vector system is provided that comprises a first vector. The first vector comprises a first sequence encoding a first component of a DNA sequence modifying complex. The first vector also comprises a second sequence encoding a second component of the DNA sequence modifying complex. There is also a first promoter operably linked to the first sequence encoding the first component of the DNA sequence modifying complex. There is also a second promoter operably linked to the second sequence encoding the second component of the DNA sequence modifying complex, wherein the DNA modifying enzyme complex modifies an endogenous copy of an essential gene. The two-vector system also comprises a second vector that comprises a rescue transgene sequence; a rescue transgene promoter operably linked to the rescue transgene sequence; and optionally, one or more cargo sequences.

In some embodiments, a two-vector system is provided that comprises a first vector comprising: a first sequence encoding a first component of a DNA sequence modifying complex, a first promoter operably linked to the first sequence encoding the first component of the DNA sequence modifying complex a rescue transgene sequence; a rescue transgene promoter operably linked to the rescue transgene sequence; and optionally, one or more cargo sequences. The two-vector system also comprises a second vector that comprises a second sequence encoding a second component of the DNA sequence modifying complex; a second promoter operably linked to the second component of the DNA sequence modifying complex, wherein the DNA modifying enzyme complex modifies an endogenous copy of an essential gene.

In some embodiments, a two-vector system comprises a first vector that comprises a DNA sequence modifying enzyme; and a first promoter operably linked to the DNA sequence modifying enzyme, wherein the DNA modifying enzyme complex modifies an endogenous copy of an essential gene. The two-vector system also comprises a second vector that comprises a rescue transgene sequence; a rescue transgene promoter operably linked to the rescue transgene sequence; and optionally, one or more cargo sequences.

In some embodiments, the “distance” is defined in terms of probability of recombination between the two vectors during each replication cycle. Without being limited by any particular theory, a 50% probability of recombination is equivalent to 50 map units or greater i.e., being equivalent to independent segregation. In some embodiments, the distance ranges from about 50 map units to about 100 map units. In some embodiments, the distance is about 50, 55, 60, 65, 70, 75, 80, 85, 90, 95, or 100 map units, or a value within a range defined by any two of the aforementioned values. In some embodiments the distance is less than 50 map units. In some embodiments the distance ranges from about 0 map unit to about 50 map units. In some embodiments, the distance is about 0, 1, 5, 10, 15, 20, 25, 30, 35, 40, 45, or 50 map units, or a value within a range defined by any two of the aforementioned values.

In some embodiments, the DNA sequence modifying enzyme is encoded by a single gene, and the Cargo/Rescue transgenes as disclosed herein can also be located at some distance from this gene on the same chromosome, or on different chromosomes. In some embodiments, the DNA sequence modifying enzyme is encoded by two tightly linked genes, and the Cargo/Rescue transgenes can also be located at some distance from these genes either on the same chromosome, or on different chromosomes. In some embodiments a first component of a DNA sequence modifying complex, and the Cargo/Rescue transgenes can also be located together, but at some distance from a second gene encoding a second component of the DNA sequence modifying enzyme on the same chromosome, or on different chromosomes. These embodiments create gene drive elements known as two locus ClvR. These are distinguished from those discussed above in which all components are tightly linked at a single locus. In the case of two locus ClvR (e.g., involving a two-vector system), ClvR components are on two different chromosomes, and segregate independently at meiosis, or they are located on the same chromosome at some distance from each other, such that recombination separates them at some frequency less than 50% of the time. This results in some gametes carrying the Cargo/Rescue but not Cas9/gRNA, others carrying Cas9/gRNA alone, and others carrying both transgene cassettes. The fate of these gametes in progeny (dead or alive) depends on when sequence modification occurs (in the germline alone or in somatic cells as well), and the presence or absence of the Cargo/Rescue. In short, the fates of the Cargo/Rescue and Cas9/gRNA components are dissociated because they do not always travel together through meiosis.

Without being limited by any particular theory, an implication of this behavior is that while with each two locus scenario the frequency of the Cargo/Rescue can increase in the population as compared to the non Cargo/Rescue bearing homologous chromosome (notwithstanding any limitations imposed by fitness costs associated with carrying the Cargo/Rescue cassette), the frequency of Cas9/gRNA (two locus version 1) or the Cas9/gRNA component not linked to the Cargo/Rescue (two locus version 2 and 3) will decrease over time since they sometimes find themselves in individuals who carry no functional copies of the essential gene, and are therefore dead. Since it is the presence of both Cas9 and gRNAs that leads to selection (indirectly, through the creation of LOF alleles of the essential gene) for the presence of the Cargo/Rescue, this means that in two locus ClvR the strength of drive (the ability create LOF alleles which select for Cargo/Rescue-bearing chromosomes and against their wildtype counterparts) wanes over time. Thus, two locus ClvR results in drive that is ultimately self-limiting, rather than self-sustaining, as is the case with single locus ClvR. Importantly, all the components of two locus ClvR already exist. It is just that the components have been rearranged in terms of their chromosomal location.

In some embodiments, the two-vector versions of Clvr, using the components described herein, and the arrangements of components described herein, can also be implemented in formats in which ClvR components are located on the same chromosome, at some distance less than 50 map units from each other. In single locus ClvR the components are tightly linked, with very little or no recombination occurring between Cargo/Rescue and Cas9/gRNA. This makes drive strong and constant, since cleavage activity is always linked to the Cargo/Rescue. In the two locus versions of ClvR described above, Cargo/Rescue and Cas9/gRNA recombine freely with each other since they are on separate chromosomes. This is equivalent to a map distance of 50 map units or greater for two loci on the same chromosome (effectively unlinked). In considering these two extremes it is important to note that versions of two locus ClvR can also be created using the same procedures, with Cargo/Rescue and Cas9 components (either together or being separated such that one is linked to the Cargo/Rescue and one is not) being located on the same chromosome at something less than 50 map units distance. In this scenario, when individuals carrying both constructs on the same chromosome are released into a population, drive will initially be strong, reflecting linkage between the two sets of components (they travel together on the same chromosome more often than not). However, as recombination between the components occurs over subsequent generations, the loci will separate, with the rate of separation being dependent on the distance between the loci. Ultimately, recombination will create a situation identical to that observed with unlinked two locus ClvR, in which the two loci are in what is referred to as linkage equilibrium. The important point is that the smaller the recombination distance is between the two transgene cassette-bearing loci is, the longer the components will remain linked. In consequence, the strength of drive will decay more slowly than with unlinked two locus ClvR. It will start as strong as that of single locus Clvr. Recombination will slowly (depending on the distance between the loci) break up this association, resulting in drive with the self-limiting characteristics of unlinked two locus ClvR.

Without being limited by any particular theory, versions of two locus ClvR with linkage are unique because they provide a method for titrating the strength of what is ultimately a self-limiting drive simply by changing the location of the two components on the same chromosome, with the strength and duration of drive being direct function of the degree of linkage: two locus ClvR with closely linked loci will have stronger drive (be able to spread more quickly and in the face of greater fitness costs), and drive for more generations, than will happen for two locus ClvRs with linkage in which the key genes are located farther apart. However, drive will ultimately be limited, as recombination occurs and the alleles approach linkage equilibrium.

In some embodiments, a two-vector system comprises a first vector comprising a DNA sequence modifying enzyme; a first promoter operably linked to the DNA sequence modifying enzyme, wherein the DNA modifying enzyme modifies an endogenous copy of an essential gene; and a second vector comprising a rescue transgene sequence; a rescue transgene promoter operably linked to the rescue transgene sequence; and optionally, one or more cargo sequences. Without being limited by any particular theory, the situation applies when the DNA sequence modifying complex is a base editor or an enzyme that does not require, for example, a guide RNA to modify an endogenous copy of an essential gene.

In some embodiments, a two-vector system comprises a first vector comprising a first sequence encoding a first component of a DNA sequence modifying complex; a second sequence encoding a second component of the DNA sequence modifying complex; a first promoter operably linked to the first sequence encoding the first component and a second sequence encoding the second component of the DNA sequence modifying complex, a second promoter operably linked to the second sequence encoding the second component, wherein the DNA modifying enzyme complex modifies an endogenous copy of an essential gene; and a second vector comprising a rescue transgene sequence; a rescue transgene promoter operably linked to the rescue transgene sequence; and optionally, one or more cargo sequences.

In some embodiments, a two-vector system comprises a first vector comprising a first sequence encoding a first component of a DNA sequence modifying complex, a first promoter operably linked to the first sequence encoding the first component of the DNA sequence modifying complex, a rescue transgene sequence; a rescue transgene promoter operably linked to the rescue transgene sequence; and optionally, one or more cargo sequences; and a second vector comprising a second sequence encoding a second component of the DNA sequence modifying complex; a second promoter operably linked to the second component of the DNA sequence modifying complex, wherein the DNA modifying enzyme complex modifies an endogenous copy of an essential gene. In some embodiments of the two-vector system, the first vector comprises the second sequence encoding the second component of the DNA sequence modifying complex, and the second vector comprises the first sequence encoding the first component of a DNA sequence modifying complex.

In some embodiments the gene drive disclosed herein is an alternative form of gene drive that utilizes Cas9 or other nucleases to bring about cleavage and repair of an essential gene that does not involve or require homing, though homing can potentially contribute to drive. This form of gene drive can also make use of base editing enzymes such as adenosine or cytosine deaminase to modify specific bases to create non-functional versions of an essential gene. It can also use Search and Replace Prime editing, which uses a Cas9 nickase linked to a reverse transcriptase, and one or more modified gRNAs to introduce base changes or insertions or deletions. Without being limited by any particular theory, the mechanism involves the DNA sequence modifying enzyme such as Cas9 and a set of gRNAs targeting an essential gene for cleavage, (or a sequence targeted base editor) located at one position in the genome, with a recoded version of the target essential gene that is immune to modification, along with any associated cargo transgenes, located at another position in the genome. This gene drive method is known as two locus ClvR, version 1. In some embodiments, individuals carrying one or both of these vectors bring about modification of the sequence of one or more copies of the endogenous version of the essential gene such that it is no longer functional. Individuals who end up inheriting only non-functional versions of the essential gene die or are sterile, while those that carry one or more copies of the rescue transgene and cargo, will survive and/or be fertile. Over multiple generations this behavior is predicted to result in the spread of the construct/vector into the population at the expense of the wild types version of the same chromosome. However, drive is ultimately limited in time (generations), and thus space (drive over generations in the presence of migration), and therefore allows for the possibility of reversal through dilution with wild types.

In some embodiments the gene drive disclosed herein is an alternative form of gene drive that utilizes Cas9 or other nucleases to bring about cleavage and repair of an essential gene that does not involve or require homing, though homing can potentially contribute to drive. This form of gene drive can also make use of base editing enzymes such as adenosine or cytosine deaminase to modify specific bases to create non-functional versions of an essential gene. It can also use Search and Replace Prime editing, which uses a Cas9 nickase linked to a reverse transcriptase, and a modified gRNA to introduce base changes or insertions or deletions. Without being limited by any particular theory, the mechanism involves a first component of the DNA sequence modifying enzyme such as Cas9 and a set of gRNAs targeting an essential gene for cleavage, (or a sequence targeted base editor) located at one position in the genome, with a recoded version of the target essential gene that is immune to modification, along with any associated cargo transgenes, and a second component of the DNA sequence modifying enzyme, located at another position in the genome. This gene drive method is known as two locus ClvR, version 2 and version. In some embodiments, individuals carrying one or both of these vectors bring about modification of the sequence of one or more copies of the endogenous version of the essential gene such that it is no longer functional. Individuals who end up inheriting only non-functional versions of the essential gene die or are sterile, while those that carry one or more copies of the rescue transgene and cargo, will survive and/or be fertile. Over multiple generations this behavior is predicted to result in the spread of the construct/vector into the population at the expense of the wild types version of the same chromosome. However, drive is ultimately limited in time (generations), and thus space (drive over generations in the presence of migration), and therefore allows for the possibility of reversal through dilution with wild types.

### Genome Modifying Systems

In some embodiments, the method of gene drive described herein is agnostic as to the mechanism by which sequence modification-dependent inactivation of the essential gene is brought about. It can involve cleavage and error-prone repair, as discussed above. It can also involve the use of base editing enzymes known from the literature. It can also utilize other DNA modifying enzymes such as sequence targeted transposases, recombinases, integrases, topoisomerases, or other enzymes that can be targeted to specific sequences in DNA to bring about sequence changes. Importantly, the exact nature of the sequence changes brought about is not critical since there are many ways of rendering nonfunctional or less functional any particular gene through sequence modification. In some embodiments, the method involves expressing an element that modifies an essential gene to render it less functional or non-functional such that the population that inherits the modified essential gene (but not a rescue gene) declines.

In some embodiments, endonucleases may be used to generate double-strand DNA breaks (DSBs) and activate genome repair pathways. These DSB repair pathways may repair the break cleanly, i.e., without altering the starting sequence, or, alternatively, induce a mutation through an error in repair. In some embodiments, genome editing is used to insert, delete, or substitute one or more base pairs at one or any combination of genetic loci. In some embodiments, a genome editing technique is used to create a mutation, for example, a point mutation or single nucleotide polymorphism.

In some embodiments the DSB repair pathway is non-homologous end-joining (NHEJ) or microhomology mediated end joining (MMEJ). During NHEJ, any nucleotide overhangs on the break ends are either resected or filled to form blunt ends that are ligated. During MMEJ, the break ends are processed to reveal overhangs comprising microhomology sequences that are then ligated together. The insertions or deletions resulting from the terminal end processing in both the NHEJ and MMEJ pathways can be referred to as indels. In some embodiments, the NHEJ or MHEJ that occurs can be relied upon to introduce a genome modification including, but not limited to, a silent mutation, a neutral mutation, a missense mutation, a nonsense mutation, or a frameshift mutation.

In other embodiments, the DSB repair pathway is homologous recombination (HR). During HR, a DSB is repaired using a template with sequences with homology to the DNA flanking the break, i.e., a homologous chromosome. In plant genome editing, a linear DNA polynucleotide flanked by sequences (e.g., of 50 base pairs or more) homologous to those flanking a targeted genomic locus, may be introduced into the genome when a DSB is repaired by HR. In some embodiments, this approach is used to introduce, substitute, or delete a DNA sequence at a genomic locus. Any DNA sequence of interest may be introduced, deleted, or substituted. An introduced or substituted DNA sequence may encode an RNA molecule with a specific activity or function, a DNA molecule with a specific activity or function (e.g., encoding a polypeptide, representing a detectable marker, etc.), a DNA molecule comprising cis-regulatory elements, or a DNA molecule encoding a polypeptide, a motif thereof, or domain thereof. In some embodiments, the nucleic acid encoding the linear DNA sequence that will act as the HR template is encoded by an expression vector. In some embodiments, the nucleic acid encoding the linear DNA sequence of interest is encoded by a DNA sequence separate from the expression vector. For example, and without limitation, the nucleic acid encoding a DNA sequence of interest may be a linear DNA polynucleotide that is co-transformed with an expression vector.

In some embodiments, single-strand breaks or “nicks” are introduced into the target DNA sequence. As used herein, the term “single-strand break inducing agent” or “nickase” refers to any agent that can induce a single-strand break (SSB) in a DNA molecule. In some embodiments two SSBs are introduced into the target DNA to generate a DSB. These breaks may also be repaired by HR, NHEJ, or MMEJ. In some embodiments, sequence modifications occur at or near the SSB sites, which can include deletions or insertions that result in modification of the nucleic acid sequence, or integration of exogenous nucleic acids by HR or NHEJ.

In one aspect, a “modification” comprises the insertion of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In another aspect, a “modification” comprises the deletion of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In a further aspect, a “modification” comprises the inversion of at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In still another aspect, a “modification” comprises the substitution of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In some embodiments, a “modification” comprises the substitution of an “A” for a “C,” “G” or “T” in a nucleic acid sequence. In some embodiments, a “modification” comprises the substitution of an “C” for an “A,” “G” or “T” in a nucleic acid sequence. In some embodiments, a “modification” comprises the substitution of a “G” for an “A,” “C” or “T” in a nucleic acid sequence. In some embodiments, a “modification” comprises the substitution of a “T” for an “A,” “C” or “G” in a nucleic acid sequence. In some embodiments, a “modification” comprises the substitution of a “C” for an “U” in a nucleic acid sequence. In some embodiments, a “modification” comprises the substitution of a “G” for an “A” in a nucleic acid sequence. In some embodiments, a “modification” comprises the substitution of an “A” for a “G” in a nucleic acid sequence. In some embodiments, a “modification” comprises the substitution of a “T” for a “C” in a nucleic acid sequence.

In some embodiments, genome editing as described herein may encompass techniques that employ methods of targeting endonucleases to one or more genetic loci. In several embodiments, genome editing is accomplished by one or more of a variety of engineered nucleases. In several embodiments, restriction enzymes are used, particularly when double strand breaks are desired at multiple regions. In several embodiments, a bioengineered nuclease is used. In some embodiments, one or more of a Zinc Finger Nuclease (ZFN), transcription-activator like effector nuclease (TALEN), meganuclease and/or clustered regularly interspaced short palindromic repeats (CRISPR/Cas9) system are used to specifically edit the genomic loci. The targeted endonucleases may catalyze a DSB at a target locus. Upon detecting these breaks, a cell may initiate any DSB repair pathway. In some embodiments, genome editing is carried out at more than one genomic locus simultaneously (i.e., multiplex genome engineering). In some embodiments, multiplex genome engineering may be used to remove a sequence of any size from the genome. In some embodiments, any combination and number of endonuclease targeting techniques may be used to target one or more genetic loci.

In some embodiments, genome editing as described herein may employ RNA-guided endonucleases including, but not limited to CRISPR/Cas systems. CRISPR/Cas systems have been described in U.S. Patent Application Publication Nos. 2017/0191082 and 2017/0106025, each of which are incorporated herein by reference in their entirety. In some embodiments, a site-specific genome modification enzyme provided herein can comprise any RNA-guided Cas endonuclease (non-limiting examples of RNA-guided nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, or modified versions thereof); and, optionally, the guide RNA necessary for targeting the respective nucleases.

In some embodiments, genome editing as described herein may employ Transcription Activator-Like Effector Nucleases (TALENs). TALENs have been described in U.S. Patent Application Publication Nos. 2016/0369301 and 2015/0203871 (both of which are incorporated herein by reference in their entirety) and are well known in the art. In one aspect, the nuclease is selected from a group consisting of PvuII, MutH, TevI and FokI, AlwI, MlyI, SbfI, SdaI, StsI, CleDORF, Clo051, Pept071. The term TALEN, as used herein, is broad and includes a monomeric TALEN that can cleave double stranded DNA without assistance from another TALEN. The term TALEN is also used to refer to one or both members of a pair of TALENs that work together to cleave DNA at the same site.

In some embodiments, genome engineering as described herein may employ a meganuclease. Meganucleases are characterized by their capacity to recognize and cut large DNA sequences (from 14 to 40 base pairs). The engineering of meganucleases can be more challenging than that of ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity. In several embodiments, two or more meganucleases, or functions fragments thereof, are fused to create a hybrid enzyme that recognizes a desired target sequence within the target gene.

In some embodiments, genome engineering as described herein may employ Zinc Finger Nucleases (ZFNs). ZFNs have been described in U.S. Pat. No. 9,322,006 (incorporated herein by reference in its entirety) and are well known in the art. ZFNs are synthetic proteins consisting of an engineered zinc finger DNA-binding domain fused to the cleavage domain of an endonuclease, for example, Fok1. ZFNs can be designed to cleave almost any long stretch of double-stranded DNA by the modification of the zinc finger DNA-binding domain. ZFNs form dimers from monomers composed of a non-specific DNA cleavage domain of FokI nuclease fused to a zinc finger array engineered to bind a target DNA sequence. The DNA-binding domain of a ZFN is typically composed of 3-4 zinc-finger arrays. The amino acids at positions-1, +2, +3, and +6 relative to the start of the zinc finger co-helix, which contribute to site-specific binding to the target DNA, can be changed and customized to fit specific target sequences. The other amino acids form the consensus backbone to generate ZFNs with different sequence specificities. Rules for selecting target sequences for ZFNs are known in the art. The FokI nuclease domain requires dimerization to cleave DNA and therefore two ZFNs with their C-terminal regions are needed to bind opposite DNA strands of the cleavage site (separated by 5-7 nt). The ZFN monomer can cut the target site if the two-ZF-binding sites are palindromic. The term ZFN, as used herein, is broad and includes a monomeric ZFN that can cleave double stranded DNA without assistance from another ZFN. The term ZFN is also used to refer to one or both members of a pair of ZFNs that are engineered to work together to cleave DNA at the same site.

In some embodiments, the target genome can be modified using site-specific genome modification techniques. In some embodiments, site-specific genome modification as described herein may employ any site-specific genome modification enzyme. As used herein, the term “site-specific genome modification enzyme” refers to any enzyme that can modify a nucleotide sequence in a sequence-specific manner. In some embodiments, a site-specific genome modification enzyme modifies the genome by inducing a single-strand break. In some embodiments, a site-specific genome modification enzyme modifies the genome by inducing a double-strand break. In some embodiments, a site-specific genome modification enzyme is a recombinase. In some embodiments, a site-specific genome modification enzyme is a transposase. In the present disclosure, site-specific genome modification enzymes include, but are not limited to, nucleases, endonucleases, recombinases, invertases, transposases, methytransferase, demethlylases, aminases, deaminases, helicases, and any combination thereof.

In some embodiments, the site-specific genome modification enzyme is a recombinase. Non-limiting examples of recombinases include a tyrosine and serine recombinases and coupled with a DNA recognition motif, for example, a Cre recombinase, a Gin recombinase, a Flp recombinase, and a Tnpl recombinase. In another aspect, a serine recombinase coupled with a DNA recognition motif, for example, a PhiC31 integrase, an R4 integrase, and a TP-901 integrase. In an aspect, a recombinase is tethered to a zinc-finger DNA-binding domain, or a TALE DNA-binding domain, or a Cas9 nuclease.

The Flp-FRT site-directed recombination system comes from the 2μ plasmid from the baker's yeast Saccharomyces cerevisiae. In this system, Flp recombinase (flippase) recombines sequences between flippase recognition target (FRT) sites. FRT sites comprise 34 nucleotides. Flp binds to the “arms” of the FRT sites (one arm is in reverse orientation) and cleaves the FRT site at either end of an intervening nucleic acid sequence. After cleavage, Flp recombines nucleic acid sequences between two FRT sites.

Cre-lox is a site-directed recombination system derived from the bacteriophage P1 that is similar to the Flp-FRT recombination system. Cre-lox can be used to invert a nucleic acid sequence, delete a nucleic acid sequence, or translocate a nucleic acid sequence. In this system, Cre recombinase recombines a pair of lox nucleic acid sequences. Lox sites comprise 34 nucleotides, with the first and last 13 nucleotides (arms) being palindromic. During recombination, Cre recombinase protein binds to two lox sites on different nucleic acids and cleaves at the lox sites. The cleaved nucleic acids are spliced together (reciprocally translocated) and recombination is complete. In another aspect, a lox site provided herein is a loxP, lox 2272, loxN, lox 511, lox 5171, lox71, lox66, M2, M3, M7, or M11 site.

In another aspect, the site-specific genome modification enzyme is a dCas9-recombinase fusion protein. As used herein, a “dCas9-recombinase fusion protein” is a dCas9 with a protein fused to the dCas9 in such a manner that the recombinase is catalytically active on the DNA. In some embodiments, dCas9 may be fused with the catalytic domain of any enzyme such that the catalytic domain is catalytically active on DNA. In another aspect, a DNA transposase is attached to a DNA binding domain for example, a TALE-piggyBac and TALE-Mutator.

In some embodiments, the DNA sequence modifying enzyme is a nuclease. Non-limiting examples of nucleases include Flap endonucleases, restriction endonucleases (e.g., F-EcoT5I, F-EcoT5II, F-EcoT5IV, F-SceI, F-TevI, F-TevII, I-AchMI, I-AniI, I-BasI, I-Bmol, I-Bth0305I, I-BthII, I-BthORFAP, I-Ceul, I-Chul, I-Cpal, I-CpaII, I-Crel, I-CsmI, I-CvuI, I-DdiI, I-DmoI, I-GpiI, I-Gzel, I-HjeMI, I-Hmul, I-HmuII, I-LlaI, I-LtrI, I-LtrWI, I-MpeMI, I-MsoI, I-NanI, I-NitI, I-NjaI, I-Onul, I-PakI, I-PanMI, I-PnoMI, I-PogTE7I, I-PorI, I-PpoI, I-ScaI, I-SceI, I-SceII, I-SceIII, I-SceVI, I-SpomI, I-SscMI, I-Ssp6803I, I-TevI, I-TevII, I-TevIII, I-TslI, I-TslWI. AY76, I-Vdi 141I, PI-Aval, PI-BciPI, PI-HvoWI, PI-MleSI, PI-Mtul, PI-PkoI, PI-PkoII, PI-PspI, PI-SceI, PI-TfuI, PI-TfuII, PI-TliI, PI-TliII, PI-TmaI, PI-TmaKI), Cas9, and Cas9-like enzymes (including but not limited to CPf1, C2c1, C2c2, and C2c3 (Shmakov et. al. 2015, Shmakov et. al. 2017, Koonin et. al. 2017-1, Koonin et. al. 2017-2), ZFNs, MegaTALs, TALENS, HEGs, meganucleases, etc.

In some embodiments, DNA modifications are achieved through cleavage by site-specific nucleases. Without being limited by any particular theory, it should be understood that equivalent effects can be obtained through the use of any enzyme that brings about modification of a target DNA sequence. Non-limiting examples include cytosine and adenine base changes brought about through the targeted use of deaminases and site-specific integrases. It can also use Search and Replace Prime editing, which uses a Cas9 nickase linked to a reverse transcriptase, and a modified gRNA to introduce base changes or insertions or deletions.

In some embodiments, the nuclease cleaves the endogenous copy of the essential gene. In some embodiments, the nuclease generates one or more double strand breaks in the endogenous copy of the essential gene. In some embodiments, the nuclease cleaves and generates one or more double strand breaks in the endogenous copy of the essential gene. In some embodiments, the one or more double strand breaks in the endogenous copy of the essential gene are staggered. In some embodiments, the one or more double strand breaks in the endogenous copy of the essential gene are not staggered. In some embodiments, the nuclease cleaves and generates one or more single strand breaks in the endogenous copy of the essential gene.

In some embodiments, the one or more double strand breaks (DSBs) are repaired. In some embodiments, the one or more DSBs are repaired to create an altered sequence of the essential gene. In some embodiments, the one or more DSBs are repaired by one or more of non-homologous end joining (NHEJ), microhomology-mediated end joining (MMEJ), homologous recombination (HR), complete HR, and incomplete HR. In some embodiments, the altered sequence comprises substitutions, insertions, deletions, frame-shifts, or a combination thereof.

In some embodiments, the DNA sequence modifying enzyme is a base editor. Non-limiting examples of a base editor include cytosine deaminase, and adenine deaminases.

In some embodiments, the base editor creates one or more base changes in endogenous copy of the essential gene. In some embodiments, the one or more base changes comprise transitions, transversions, or both. In some embodiments, the one or more base changes occur due to tautomerism, depurination, deamidation, or a combination thereof. In some embodiments, the one or more base changes creates an altered sequence of the essential gene. In some embodiments, the one or more base changes comprise one or more point mutations in the endogenous copy of the essential gene. In some embodiments, the one or more point mutations comprise frameshift mutation, nonsense mutation, missense mutation, neutral mutation, silent mutation, or a combination thereof.

In some embodiments, the DNA sequence modifying enzyme is a Search and Replace Prime editor, which uses a Cas9 nickase linked to a reverse transcriptase, and a modified gRNA to introduce base changes or insertions or deletions.

In some embodiments, the Search and Replace Prime editor creates one or more base changes in endogenous copy of the essential gene. In some embodiments, the one or more base changes comprise transitions, transversions, or both. In some embodiments, the one or more base changes occur due to tautomerism, depurination, deamidation, or a combination thereof. In some embodiments, the one or more base changes create an altered sequence of the essential gene. In some embodiments, the one or more base changes comprise one or more point mutations in the endogenous copy of the essential gene. In some embodiments, the one or mutations comprise frameshift mutation, nonsense mutation, missense mutation, small deletions or additions, neutral mutation, silent mutation, or a combination thereof.

In some embodiments, the promoter of the first gene expresses within females such that the DNA-modifying enzyme produced by the first gene is deposited into eggs and can modify target sequences inherited from a father who lacks the vector. This activity, while unnecessary, for the majority of cases wherein this drive method successfully replaces a population, results in more rapid population replacement than without, for a given fitness cost and/or introduction frequency. Where the DNA-modifying enzyme is a version of Cas9 or a Cas9-related enzyme (guided to a target sequence by a guide RNA), both Cas9 and any and all associated gRNAs are deposited into the eggs of such females together to enable modification of alleles inherited from a non-vector bearing male.

In some embodiments, there is paternal carryover of the DNA modifying enzyme, allowing for modification of alleles inherited from the mother, even in those who have not inherited the vector.

Several embodiments relate to promoting DNA recombination by providing a site-specific genome modification enzyme to a plant cell. In some embodiments, recombination is promoted by providing a strand separation inducing reagent. In one aspect, the site-specific genome modification enzyme is selected from an endonuclease, a recombinase, an invertase, a transposase, a helicase or any combination thereof. In some embodiments, recombination occurs between B chromosomes. In some embodiments, recombination occurs between a B chromosome and an A chromosome.

Several embodiments relate to promoting integration of one or more DNAs of interest by providing a site-specific genome modification enzyme. In some embodiments, integration of one or more DNAs of interest is promoted by providing a strand separation inducing reagent. In one aspect, the site-specific genome modification enzyme is selected from an endonuclease, a recombinase, a transposase, a helicase or any combination thereof. Any DNA sequence can be integrated into a target site of a chromosome sequence by introducing the DNA sequence and the provided site-specific genome modification enzymes. Any method provided herein can utilize any site-specific genome modification enzyme provided herein.

Several embodiments relate to a method and/or a composition provided herein comprising at least one, at least two, at least three, at least four, at least five, at least six, at least seven, at least eight, at least nine, or at least ten site-specific genome modification enzymes. In yet another aspect, a method and/or a composition provided herein comprises at least one, at least two, at least three, at least four, at least five, at least six, at least seven, at least eight, at least nine, or at least ten polynucleotides encoding at least one, at least two, at least three, at least four, at least five, at least six, at least seven, at least eight, at least nine, or at least ten site-specific genome modification enzymes.

### Vectors

Vectors for transformation may include any vector that is generally known in the art. Some non-limiting examples of the vectors that can be used are plasmids, cosmids, YACs (yeast artificial chromosomes), BACs (bacterial artificial chromosomes) or any other suitable cloning system, as well as fragments of DNA therefrom. Thus, when the term “vector” or “expression vector” is used, all of the foregoing types of vectors, as well as nucleic acid sequences isolated therefrom, are included. It is contemplated that utilization of cloning systems with large insert capacities will allow introduction of large genetic sequences comprising more than one selected gene. In accordance with the disclosure, this could be used to introduce genetic material corresponding to an entire biosynthetic pathway into an organism. Introduction of such sequences may be facilitated by use of bacterial or yeast artificial chromosomes (BACs or YACs, respectively), or even plant artificial chromosomes. For example, the use of BACs for Agrobacterium-mediated transformation was disclosed by Hamilton et al. (1996).

FIG. 1A shows an embodiment of ClvR element design and principle according to the present disclosure (Example 1). In some embodiments, the vector comprises a gene encoding a DNA sequence modifying enzyme. In some embodiments, the DNA modifying enzyme modifies the sequence of an endogenous copy of an essential gene.

In some embodiments, the invention discloses one or more vectors that comprise a promoter operably linked to the gene encoding the DNA sequence modifying enzyme. In some embodiments, the gene encoding a DNA sequence modifying elements is a first gene. In some embodiments, the gene encoding a DNA sequence modifying elements is a second gene. In some embodiments, the promoter operably linked to the gene encoding the DNA sequence modifying enzyme is a first promoter. In some embodiments, the promoter operably linked to the gene encoding the DNA sequence modifying enzyme is a second promoter. In some embodiments, the gene encoding a DNA sequence modifying elements is operably linked to one or more additional regulatory elements.

In some embodiments, the one or more vector(s) further comprises a gene encoding a rescue transgene. In some embodiments, the gene encoding a rescue transgene is a first gene. In some embodiments, the gene encoding a rescue transgene is a second gene. In some embodiments of the one or more vector(s), a promoter is operably linked to the rescue transgene is a first promoter. In some embodiments of the one or more vector(s), a promoter is operably linked to the rescue transgene is a second promoter. In some embodiments, the gene encoding a rescue transgene is operably linked to one or more additional regulatory elements. In some embodiments, the rescue gene is a recoded version of essential gene that is targeted. In some embodiments, the rescue gene is of unrelated sequence, wherein the rescue transgene encodes a protein that is functionally equivalent to a protein encoded by the essential gene or is otherwise able to make up for the loss of activity of the endogenous essential gene.

In some embodiments, a two-vector system is provided that comprises: a first vector. The first vector comprises: a first sequence encoding a first component of a DNA sequence modifying complex; a second sequence encoding a second component of the DNA sequence modifying complex; a first promoter operably linked to the first sequence encoding the first component of the DNA sequence modifying complex, a second promoter operably linked to the second sequence encoding the second component of the DNA sequence modifying complex. The DNA modifying enzyme complex modifies an endogenous copy of an essential gene. The system comprises a second vector that comprises a rescue transgene sequence; a promoter operably linked to the rescue transgene that requires binding by the DNA sequence modifying complex for transcription of the rescue transgene; and optionally, one or more cargo sequences.

In some embodiments a two-vector system is provided that comprises a first vector that comprises a first sequence encoding a first component of a DNA sequence modifying complex, a first promoter operably linked to the first sequence encoding the first component of the DNA sequence modifying complex, a rescue transgene sequence; a promoter operably linked to the rescue transgene that requires binding by the DNA sequence modifying complex for transcription of the rescue transgene; and optionally, one or more cargo sequences. The two-vector system further comprises a second vector that comprises a second sequence encoding a second component of the DNA sequence modifying complex; and a second promoter operably linked to the second component of the DNA sequence modifying complex. The DNA modifying enzyme complex modifies an endogenous copy of an essential gene. In some embodiments, the first vector comprises the second sequence encoding the second component of the DNA sequence modifying complex, and the second vector comprises the first sequence encoding the first component of a DNA sequence modifying complex.

In some embodiments, the one or more vector(s) optionally comprises one or more cargo sequences. In some embodiments, a cargo sequence is a nucleic acid. In some embodiments, the vector is configured to be positioned in a chromosome. In some embodiments, the vector is configured to be positioned in an extra-chromosomal element. Non-limiting examples of cargo genes include are sequences encoding antibodies against Plasmodium, the causal agent of malaria (Isaacs et. al. 2011, Hollingdale et. al. 1984, and Li et. al. 2005), or non-coding RNAs to bring about cleavage of the dengue virus RNA genome (Yen et. al. 2018, Franz et. al. 2006, Mathur et. al. 2010, Travanty et. al. 2004, and Castillo et. al. 2016). In some embodiments, cargo genes encode genes that confer resistance or susceptibility to disease or toxins or alter phenotypes that promote or inhibit survival in the wild, or that result in increased fitness cost to carriers, resulting in population suppression. In some embodiments, cargo genes encode proteins that confer resistance to herbicides and/or other compositions that are applied to plants. In some embodiments, cargo genes encode proteins that confer increased fitness to plants in specific environments. One of skill in the art can readily identify one or more genes depending upon the desired trait to be exhibited in the cell or organism developed from the cell.

In some embodiments, the one or more cargo genes comprise foreign gene sequences, or one or more alleles of an endogenous chromosomal or extra-chromosomal gene.

In some embodiments the cargo genes comprises one or more alleles of an endogenous chromosomal or extra-chromosomal gene to which the vector has been linked through nearby insertion on the chromosome or extra-chromosomal element that carries the endogenous allele of interest.

In some embodiments, the cargo genes can be physically part of the vector prior to its insertion in a chromosomal or an extra-chromosomal element. In some embodiments, the cargo can be a chromosomal/extra-chromosomal allele of a gene that becomes linked to the vector after the insertion of the vector near that allele. In some embodiments, a fraction of the cargo can be physically part of the vector prior to its insertion in a chromosomal or an extra-chromosomal element, and a remainder of the cargo can be a chromosomal/extra-chromosomal allele of a gene that becomes linked to the vector after the insertion of the vector near that allele. In some embodiments, the cargo gene does not have to be a part of the vector, i.e., in some embodiments, the cargo is optional and can be physically part of the vector prior to its insertion in a chromosomal or an extra chromosomal element. In some embodiments, the cargo gene does not have to be a part of the vector, i.e., in some embodiments, a fraction of the cargo can optionally be physically part of the vector prior to its insertion in a chromosomal or an extra chromosomal element, and a remainder of the cargo can be a chromosomal/extra-chromosomal allele of a gene that becomes linked to the vector after the insertion of the vector near that allele.

In some embodiments, the size of the one or more cargo sequences ranges from about is about 0.5 kb to about 500 kb. In some embodiments, the size ranges from about 1 kb to about 1000 kb. In some embodiments, the size ranges from about 5 kb to about 5000 kb. In some embodiments, the size ranges from about 10 kb to about 10000 kb. In some embodiments, the size is about 0.1, 0.5, 1, 5, 10, 25, 50, 75, 100, 250 500, 750 1000, 2500, 5000, 7500, or 10000 kb.

In some embodiments, the one or more vector(s) is configured to be positioned in a chromosome and an extra chromosomal element. In some embodiments, the one or more vector(s) is configured to be positioned in a chromosome but not in an extra chromosomal element. In some embodiments, the one or more vector(s) is configured to be positioned in an extra chromosomal element but not in a chromosome. In some embodiments, the one or more vectors are configured such that the gene encoding a DNA sequence modifying enzyme, the enhancer/promoter operably linked to the gene encoding the DNA sequence modifying enzyme, the promoter and/or the nucleic acid encoding one or more gRNAs are positioned on a different chromosome or an extra-chromosomal element than gene encoding a rescue transgene. In some embodiments, the one or more vectors are configured such that the gene encoding a DNA sequence modifying enzyme, the enhancer/promoter operably linked to the gene encoding the DNA sequence modifying enzyme, the promoter and/or the nucleic acid encoding one or more gRNAs are positioned on the same chromosome at a distance from the rescue transgene. In some embodiments, the distance is less than 50 map units.

In some embodiments, the rescue transgene is a recoded copy of the essential gene. In some embodiments, when the rescue transgene is a recoded copy of the essential gene, the protein encoded by the recoded copy of the essential gene (recoded protein) is about 90% to about 99.9% identical to protein encoded by the endogenous copy of the essential gene (endogenous protein). In some embodiments, the recoded protein is about 90, 91, 92, 93, 94, 95, 96, 97, 98, 99, 99.1, 99.2, 99.3, 99.4, 99.5, 99.6, 99.7, 99.8, or 99.9% to the endogenous protein.

In some embodiments, the rescue transgene is a gene of unrelated sequence. In some embodiments, when the rescue transgene is a gene of unrelated sequence, the protein encoded by the recoded copy of the essential gene (recoded protein) is functionally equivalent to the protein encoded by the endogenous copy of the essential gene (endogenous protein). In some embodiments, the DNA sequence modifying enzyme does not modify the rescue transgene.

In some embodiments, the chromosome in which the vector is positioned is one or more of an autosome, X chromosome, Y chromosome, Z chromosome, W chromosome or supernumerary chromosome. In some embodiments, the vector is positioned in one or more combinations of an autosome, X chromosome, Y chromosome, Z chromosome, W chromosome or supernumerary chromosome. For example, in some embodiments, the vector is positioned in an autosome and an X chromosome, in some embodiments, the vector is positioned in an autosome and a Y chromosome, in some embodiments, the vector is positioned in an autosome and a supernumerary chromosome, in some embodiments, the vector is positioned in an X chromosome and a Y chromosome, in some embodiments, the vector is positioned in an X chromosome and a supernumerary chromosome, in some embodiments, the vector is positioned in an Y chromosome and a supernumerary chromosome, and in some embodiments, the vector is positioned in an autosome, X chromosome, Y chromosome, and supernumerary chromosome. In some embodiments, the vector is inserted into a gene required for diploid fertility of one sex, thereby disrupting the gene required for diploid fertility of one sex. In some embodiments, the vector is inserted next to or within a locus whose presence is sufficient for adult male or female sex determination.

In some embodiments, the vector is positioned in an extra-chromosomal element. In some embodiments, the extra-chromosomal element is a plasmid. In some embodiments, the extra-chromosomal element is a virus. In some embodiments, the extra-chromosomal element is a plasmid and a virus. In some embodiments, the vector is positioned in combinations of one or more chromosomes and one or more extra-chromosomal elements.

In some embodiments herein, the vector comprising the first gene encoding the DNA sequence modifying enzyme and the second gene encoding the rescue transgene is referred to as CLeaveR, which comprises and/or consists of two components: (1) a site-specific DNA modifying enzyme designed to alter the sequence of an endogenous gene required for survival, proliferation, fertility, or differentiation so as to render it non-functional; (2) a recoded version of the essential gene resistant to cleavage, and having reduced nucleotide identity with the endogenous. Optionally, one or more cargo sequences are present.

In some embodiments, DNA sequence modifying enzyme is, without limitation, Cas9, Cas-9-related RNA-guided nucleases, ZFNs, TALENs, homing endonucleases, restriction enzymes, natural site-specific nucleases, engineered site-specific nucleases, base editing enzymes, cytidine deaminase, and adenine deaminase.

In some embodiments, the vector further comprises one or more additional sequences. In some embodiments, the one or more additional sequences allow the vector to be positioned in the chromosome. In some embodiments, the one or more additional sequences allow the vector to be positioned in the extra-chromosomal element. In some embodiments, the one or more additional sequences allow the vector to be positioned in the chromosome and the extra-chromosomal element. In some embodiments, the one or more additional sequences allow the vector to be positioned in the chromosome but not the extra-chromosomal element. In some embodiments, the one or more additional sequences allow the vector to be positioned in the extra-chromosomal element but not the chromosome.

In some embodiments, the one or more additional sequences is, without limitations, regulatory sequence, transposase binding site, LTRs, transgenesis markers, recombinase binding site, a sequence with homology to a desired location on the chromosome or a sequence with homology to a desired location on the extra-chromosomal element, or combinations thereof.

In some embodiments, the size of the one or more regulatory sequences ranges from about is about 0.5 kb to about 500 kb. In some embodiments, the size ranges from about 1 kb to about 1000 kb. In some embodiments, the size ranges from about 5 kb to about 5000 kb. In some embodiments, the size ranges from about 10 kb to about 10000 kb. In some embodiments, the size is about 0.1, 0.5, 1, 5, 10, 25, 50, 75, 100, 250 500, 750 1000, 2500, 5000, 7500, or 10000 kb.

In some embodiments, the vector further comprises one or more additional sequences, wherein the one or more additional sequences serve as dominant marker genes that allow individuals carrying the vector to be easily identified either visually, as with expression of a fluorescent protein, or by virtue of surviving a negative selection procedure, as with expression of a gene that encodes resistance to a toxin (such as an antibiotic, insecticide, herbicide), in the presence of the toxin. In some embodiments, the vector comprises one or more sequences that encode marker proteins that can be expressed under the control of suitable regulatory elements. Non-limiting examples of marker proteins include dsRed, GFP, EGFP, CFP, ECFP, BFP, EBFP, mHoneydew, mBanana, mOrange, tdTomato, mTangering, mStrawberry, mCherry, mGrape1, mGrape2, mRaspberry, mPlum, fast RFP, YFP or EYFP, and can be chosen by one of skilled in the art according to need. Fluorescent marker protein can be visualized by illuminating with a suitable excitatory wavelength and observing the fluorescence (e.g., by fluorescence microscopy). In some embodiments, a marker protein would allow for easy identification of organisms carrying the vector. In some embodiments, a marker protein would provide resistance. In some embodiments, the vector has an herbicide resistance marker. In some embodiments, the marker is basta herbidice resistance marker.

In some embodiments, the first promoter is, without limitations, a germline promoter, a male specific germline promoter, a female specific germline promoter, a cell-type specific promoter, a tissue-specific promoter, a ubiquitous promoter, a promoter activated at a specific stage of mitosis, a promoter activated at a specific stage of meiosis, or combinations thereof. In some embodiments, the enhancer or promoter is selected from the group consisting of ubiquitin-10, apetala1, agamous, DMC-1, clavata-3 and CaMV35S. In some embodiments, first promoter is an U6 promoter. In some embodiments, a second promoter is without limitations, a germline promoter, a male specific germline promoter, a female specific germline promoter, a cell-type specific promoter, a tissue-specific promoter, a ubiquitous promoter, a promoter activated at a specific stage of mitosis, a promoter activated at a specific stage of meiosis, or combinations thereof. In some embodiments, the second enhancer or promoter is selected from the group consisting of ubiquitin-10, apetala1, agamous, DMC-1, clavata-3 and CaMV35S. In some embodiments, the second promoter is independent of the first promoter. In some embodiments, second promoter is an U6 promoter.

In some embodiments, the nuclease comprises at least one nuclease domain. In some embodiments, the nuclease comprises one or more DNA binding domains. In some embodiments, the nuclease comprises at least one nuclease domain and one or more DNA binding domains. In some embodiments, the DNA binding domain is a TALE or a zinc finger. In some embodiments, the nuclease domain is provided as a single active nuclease domain, such as single chain variants of Fok1.

In some embodiments, when the nuclease is Cas9 or a Cas9-related enzyme, the vector further comprise one or more genes encoding a guide RNA. In some embodiments, Cas9 is modified and possesses increased expression and catalytic activity relative to an unmodified Cas9. In some embodiments, the modified Cas9 carries one or more introns and/or carries at least one mutation. In some embodiments, the mutation is K918N.

In some embodiments, the guide RNA enables the nuclease to target specific DNA sequences through Watson-Crick base pairing, thereby allowing targeting of very many positions in any genome. In some embodiments, the guide RNA enables the nuclease to target specific sequences within the endogenous copy of the essential gene. In some embodiments, the guide RNA enables the nuclease to target specific sequences within the protein coding region of an endogenous copy of the essential gene. In some embodiments, the guide RNA allows the nuclease to target specific sequences within the non-coding region of an endogenous copy of the essential gene. In some embodiments, the guide RNA allows the nuclease to target specific sequences outside the endogenous copy of the essential gene.

In some embodiments, when the nuclease is Cas9, the nuclease domain of Cas9 is inactivated through one or more mutations and the vector comprises a different nuclease domain. In some embodiments, the different nuclease domain is single chain variant of FokI. In some embodiments, when the DNA binding domain is a TALE, the nuclease domain is provided as a single active nuclease domain. In some embodiments, the single active nuclease domain is a single chain variant of FokI. In some embodiments of the vector, when the DNA binding domain is a TALE, the nuclease domain is provided as a single active nuclease domain, such as single chain variants of FokI (Sun and Zhao 2014).

In some embodiments of single locus and two locus ClvR the separation of a functional Rescue gene from the Cargo gene (if present) can be prevented by locating the Cargo in an intron of the Rescue. A break between the two genes followed by reciprocal end joining with the same region on the homologous chromosome could separate them. In some embodiments of single and two locus ClvR separation of a functional Rescue from the Cargo can be reduced by locating the Cargo between two transgenes whose co-expression is required to produce a functional Rescue essential enzyme, such as dihydrofolate reductase. The 5′ half of DHFR is driven by its own promoter. The 3′ half is driven by a strong ubiquitous promoter. The two domains are brought together to form an active enzyme through heterodimerization, mediated by specific domains at the N-terminus of each protein.

In some embodiments of single and two locus ClvR separation of a functional Rescue from the Cargo can be reduced by locating the Cargo between two transgenes whose co-expression is required to produce a functional Rescue protein. Here this is achieved using a two-component transcription-based system. The essential gene promoter drives the expression of a heterologous transcriptional activator such as GAL4. The Rescue transgene contains GAL4 UAS binding sites sufficient to drive GAL4-dependent expression, upstream of an otherwise promoterless, recoded Rescue transgene.

In some embodiments of single and two locus ClvR Cas9 can be made essential for Rescue function. The system thus creates tight linkage between components required for cleavage and those required for rescue. It can fail due to point mutations in Cas9 that allow target site DNA binding and transcriptional activation but that prevent cleavage, as with dead Cas9 variants used for transcriptional regulation or visualization of specific genomic loci. These will happen, but are very specific mutations, and thus any spread of dead Cas9 within the population should be delayed. An important requirement for this approach is that the essential gene be expressed in the germline at levels sufficient to bring about Cas9-dependent germline cleavage of the wildtype essential gene. Also note that unless the essential gene is only required in the germline, Cas9 will be expressed and active in some somatic tissues.

### Methods

One of ordinary skill in the art would appreciate that any of the methods disclosed herein can be performed by any of the vectors provided herein.

In some embodiments, a method of modifying a population by a vector or vectors is provided. In some embodiments, the method comprises obtaining an organism of the population. In some embodiments, one or more vectors are introduced into an individual organism of the population. In some embodiments, the organism is a plant. In some embodiments, the vector comprises a nucleic acid sequence encoding: a first gene encoding a rescue transgene; a first enhancer/promoter operably linked to a second gene encoding the DNA sequence modifying enzyme and optionally, one or more regulatory sequences; a second gene encoding a DNA sequence modifying enzyme; and a second promoter operably linked to one or more guide RNAs (gRNAs) targeting the endogenous copy of an essential gene; and one or more guide RNAs (gRNAs) targeting the endogenous copy of an essential gene. In some embodiments, the vector comprises one or more cargo sequences. In some embodiments, the DNA modifying enzyme modifies an endogenous copy of a gene essential for survival or fertilization ability of gametes from one or both sexes (an essential gene). In some embodiments, the method comprises expressing the DNA sequence modifying enzyme in a cell or cells of the organism that ultimately gives rise to a germline. The expression of the DNA sequence modifying enzyme induces one or more sequence modifications in the cell, such that the one or more sequence modifications result in the essential gene being rendered partially or wholly non-functional. In some embodiments this results in death or loss of ability to participate in fertilization if the cell lacks the rescue transgene. In some embodiments, the partially or wholly non-functional gamete is rescued by the rescue transgene, generating an altered gamete containing one or more vectors. In some embodiments, the altered gamete in which the essential gene has been rendered partially or wholly non-functional has been rescued by the rescue transgene. In some embodiments, the method comprises introducing a progeny carrying the altered gamete in an environment, wherein an increase in a frequency of the rescue transgene bearing progeny is desired relative to a frequency of a wild type individual organism in a population of the organism.

In some embodiments, the one or more vector(s) is configured to be positioned in a chromosome and an extra chromosomal element. In some embodiments, the one or more vector(s) is configured to be positioned in a chromosome but not in an extra chromosomal element. In some embodiments, the one or more vector(s) is configured to be positioned in an extra chromosomal element but not in a chromosome. In some embodiments, the one or more vectors are configured such that the gene encoding a DNA sequence modifying enzyme, the enhancer/promoter operably linked to the gene encoding the DNA sequence modifying enzyme, the promoter and/or the nucleic acid encoding one or more gRNAs are positioned on a different chromosome or an extra-chromosomal element than gene encoding a rescue transgene. In some embodiments, the one or more vectors are configured such that the gene encoding a DNA sequence modifying enzyme, the enhancer/promoter operably linked to the gene encoding the DNA sequence modifying enzyme, the promoter and/or the nucleic acid encoding one or more gRNAs are positioned on the same chromosome at a distance from the rescue transgene. In some embodiments, the distance is less than 50 map units.

In some embodiments, the organism is a diploid that produces haploid gametes. In some embodiments, the diploid undergoes meiosis to form haploid gametes before undergoing fertilization to form a new diploid. In some embodiments, the progeny is formed by mating of haploid gametes. In some embodiments, the progeny is formed by mating of haploid gametes of the same species. In some embodiments, the progeny is formed by mating of haploid gametes of two related species. In some embodiments, haploid gametes that do not comprise a rescue transgene do not survive. Therefore, over time, more and more of the population comprises vector with rescue transgene. Depending on the nature of the vector, including, for example, where vector is located and whether vector includes cargo gene, this will have a desired impact on population.

In some embodiments, the organism is a polyploid that gives rise to gametes with half adult the number of each chromosome.

In some embodiments, the organism is a plant.

In some embodiments, the method includes inserting into the cells (e.g., germline cells) of an organism, for example a plant, one or more vectors that express cargo gene(s) that renders the organism sensitive to an external stimulus. In some embodiments, the external stimulus is a chemical, compound or a condition. In some embodiments, the method sensitizes the organism to a chemical or condition which ordinarily would not be toxic to the organism. Some non-limiting examples of the chemical or condition include herbicide, pesticide or environmental condition such as temperature, salinity, light or water levels.

In some embodiments, the organism which is resistant to herbicide or pesticide before modification is sensitive to the herbicide or pesticide after modification. In some embodiments, the organism after modification may contain an enzyme or molecule that renders it susceptible to a compound or chemical. In some embodiments, the modification renders the population of an organism less harmful to other species in the environment.

In some embodiments, the organism is a weed. In some embodiments, the weed is selected from the group consisting of smooth pigweed (Amaranthus hybridus), Palmer amaranth (Amaranthus palmeri), waterhemp (Amaranthus rudis), common lambsquarters (Chenopodium album), bur cucumber (Sicyos angulatus), morning glory (Ipomeoa spp.), and trumpet creeper (Campsis radicans), henbit (Lamium amplexicaule), marestail (horsetail) [Conyza canadensis], mustards (Brassicaceae), and weedy bromes (Bromus spp.).

In some embodiments, the organism produces haploid gametes. In some embodiments, the progeny is formed by mating of haploid gametes of the same species. In some embodiments, the progeny is formed by mating of haploid gametes of two related species. In some embodiments, the resulting population may be referred to as a modified or altered population.

In some embodiments, the method includes inserting into the cells (e.g., germline cells) of an organism, for example a plant, one or more vectors that express cargo gene(s) that increase the fitness of the organism. In some embodiments, the method includes inserting into the cells (e.g., germline cells) of an organism, for example a plant, one or more vectors that renders the organism resistant to an external stimulus. In some embodiments, the external stimulus is a condition. Some non-limiting examples of the condition include environmental conditions such as temperature, stress, drought, inadequate soil fertility, nutrient deficiencies, pollution, salinity, light, water levels, presence of a weed or pest, competitor or pathogen. In some embodiments, the modification renders the organism drought resistant.

In some embodiments, a method for gene drive-mediated population modification of an organism that expresses one or more cargo gene(s) is provided. In some embodiments, the cargo gene encodes for antibodies or other molecules that protect against a pathogen that causes a disease. In some embodiments, the cargo gene encodes a resistance gene to a disease. In some embodiments, the cargo gene encodes a resistance gene to a toxin. In some embodiments, the cargo gene encodes a gene that converts an otherwise resistant organism susceptible to a disease. One of skill in the art can readily identify one or more genes depending upon the desired trait to be exhibited in the cell or organism developed from the cell.

In some embodiments, a method for gene drive-mediated population modification of an organism that expresses one or more cargo gene(s) is provided. In some embodiments, the cargo gene encodes for a protein that makes the organism susceptible to a herbicide.

In some embodiments, a method for gene drive-mediated population modification of an organism that expresses one or more cargo gene(s) is provided. In some embodiments, the cargo gene encodes for a protein that makes the organism resistant to a weed, pest or an invasive species. In some embodiments, cargo gene encodes for a protein that causes the organism to react to external stimulus in a desired way. In some embodiments the cargo gene may make a weed or invasive species susceptible to an herbicide or other chemical treatment. In some embodiments the cargo gene may reduce the fitness of a weed or invasive species, thus reducing its ability to survive in particular environments. In some embodiments, the weed or invasive species may include, but are not limited to Imperata cylindrica, Arundo donax, Cyperus esculentus, Festuca arundinacea, Microstegium vimineum, Miscanthus sinensis, Panicum repens, Phragmites australis, Phyllostachys aurea, Pseudosasa japonica, Sorghum halepense, Bambusa, Dactylis glomerata, Pennisetum clandestinim, Oryza punctata, Oryza longistaminata, Leptochloa chinensis, Nassella trichotoma, Setaria pumila ssp pallidefusca, Saccharum spontaneum, Pennisetum macrourum, Paspalum scrobiculatum, Rottboellia cochinchinensis, Ischaemum rugosum, Chrysopogon aciculatus, Digitaria velutina, Oryza rufipogon, Pennisetum pedicellatum, Pennisetum polystachion, Avena sterilis, Digitaria abyssinica, Imperata brasiliensis, Urochloa panicoides, Achnatherum brachychaetum, Aegilops cylindrica, Aegilops geniculata, Aegilops triuncialis, Agrostis stolonifera, Alopecurus myosuroides, Ammophila arenaria, Andropogon virginicus, Arrhenatherum elatius, Arthraxon hispidus, Avena barbata, Avena fatua, Axonopus fissifolius, Bromus arvensis, Bromus catharticus, Bromus racemosus, Brachypodium distachyon, Bromus diandrus, Bromus hordeaceus, Bromus hordeaceus ssp. hordeaceus, Bromus inermis, Bromus rubens, Bromus secalinus, Bromus sterilis, Bromus tectorum, Cenchrus echinatus, Cenchrus longispinus, Cenchrus spinifex, Cortaderia jubata, Cortaderia selloana, Cynodon dactylon, Cyperus difformis, Cyperus haspan, Cyperus involucratus, Cyperus iria, Cyperus prolifer, Cyperus rotundus, Dactyloctenium aegyptium, Digitaria ciliaris, Digitaria sanguinalis, Echinochloa crus-galli, Ehrharta calycina, Ehrharta erecta, Microlaena stipoides, Eleusine indica, Elymus repens, Eragrostis cilianensis, Eriochloa villosa, Heteropogon contortus, Hordeum jubatum, Holcus lanatus, Hordeum murinum ssp. leporinum, Hymenachne amplexicaulis, Hyparrhenia rufa, Imperata brevifolia, Juncus effusus, Juncus planifolius, Lolium perenne, Festuca pratensis, Melinis minutiflora, Melinis repens, Milium vernale, Muhlenbergia schreberi, Nardus stricta, Neyraudia reynaudiana, Panicum antidotale, Panicum capillare, Paspalum conjugatum, Paspalum dilatatum, Panicum miliaceum, Paspalum notatum, Paspalum urvillei, Panicum virgatum, Pennisetum glaucum, Cenchrus setaceus, Phalaris aquatica, Phalaris arundinacea, Phalaris canariensis, Phalaris minor, Phyllostachys nigra, Phleum pratense, Poa annua, Poa bulbosa, Poa compressa, Polypogon monspeliensis, Poa pratensis, Poa trivialis, Rhynchospora caduca, Sacciolepis indica, Schismus arabicus, Schismus barbatus, Schizachyrium condensatum, Secale cereale, Setaria faberi, Setaria italica, Setaria parviflora, Setaria palmifolia, Setaria pumila, Setaria verticillata, Setaria viridis, Setaria viridis var. major, Setaria viridis var. viridis, Sorghum×almum, Sorghum bicolor, Sorghum bicolor ssp. drummondii, Spartina alterniflora, Spartina anglica, Spartina densiflora, Sporobolus indicus, Spartina patens, Taeniatherum caput-medusae, Megathyrsus maximus, Urochloa mutica, Urochloa texana, Ventenata dubia, Vossia cuspidata, Vulpia myuros, Eragrostis curvula, Agrostis spp., Agrostis gigantea, Carex kobomugi, Agrostis capillaris, Lolium temulentum, Pennisetum spp., Lolium spp., Cyperus entrerianus, Anthoxanthum odoratum, Cyperus brevifolioides, Bambusa vulgaris, Hemarthria altissima, Poa nemoralis, Hordeum murinum ssp. glaucum, Miscanthus sacchariflorus, Kyllinga brevifolia, Aira caryophyllea, Luzula luzuloides, Carex longii, Carex flaccosperma, Kyllinga nemoralis, Zizaniopsis miliacea, Briza maxima, Apera interrupta, Axonopus compressus, Brachypodium sylvaticum, Bromus briziformis, Chloris barbata, Coix lacryma-jobi, Cyperus microiria, Digitaria eriantha, Digitaria insularis, Digitaria setigera, Digitaria violascens, Echinochloa colona, Eragrostis cumingii, Eragrostis lehmanniana, Eriochloa fatmensis, Festuca trachyphylla, Festuca rubra, Vulpia bromoides, Thinopyrum ponticum, Thinopyrum intermedium, Stenotaphrum secundatum, Sporobolus indicus var. capensis, Sporobolus diandrus, Garnotia stricta, Gastridium phleoides, Holcus mollis, Hordeum murinum, Juncus ensifolius, Rytidosperma bianmulare, Rytidosperma penicillatum, Polypogon viridis, Oplismenus hirtellus, Paspalum fimbriatum, Paspalum quadrifarium, Scleria lacustris, Cyperus spp., Sorghum, Setaria spp., Eremochloa ophiuroides, Paspalum vaginatum, Oxycaryum cubense, Agropyron desertorum, Agrostis avenacea, Alopecurus bladhii, Carex acutiformis, Cynosurus echinatus, Dichanthium pratensis, Bothriochloa annulatum, Dichanthium aristatum, Ehrharta longiflora, Glyceria declinata, Nassella tenuissima, Pennisetum villosum, Phyllostachys spp., Bromus diandrus var. rigidus, Lolium multiflorum, Glyceria maxima, Bambusa multiplex, Digitaria spp., Urochloa distachya, Alopecurus geniculatus, Poa pratensis ssp. pratensis, Leymus arenarius, Bromus japonicus, Pseudosasa, Tribolium obliterum, Scleria eggersiana, Juncus inflexus, Festuca filiformis, Carex pendula, Carex flacca, Phyllostachys bambusoides, Carex macrocephala, Briza minor, Dactylis glomerata ssp. glomerata, Arundinaria pygmaea, Phragmites spp., Thinopyrum junceiforme, Hordeum marinum, Miscanthus, Bromus inermis ssp. inermis var. inermis, Bromus inermis ssp. inermis, Anthoxanthum odoratum ssp. odoratum, Arrhenatherum elatius var. elatius, Hyparrhenia hirta, Nassella manicata, Scleria microcarpa, Carex disticha, Agrostis canina, Puccinellia distans, Sporobolus indicus var. pyramidalis, Bothriochloa ischaemum, Setaria pumila ssp. pumila, Phyllostachys aureosulcata, Typha laxmannii, Spartina foliosa×alterniflora, Luziola subintegra, Saccharum ravennae, Phragmites australis ssp. australis, Setaria glauca, Tripidium ravennae ssp. ravennae, Bambusa bambos, Miscanthus giganteus, Stipa capensis, Cenchrus ciliaris, Cenchrus polystachios, Rytidosperma caespitosum, Stipa miliacea var. miliacea, Pleioblastus fortunei, Dianella ensifolia, Arthraxon hispidus var. hispidus, Brachypodium sylvaticum ssp. sylvaticum, Cenchrus alopecuroides, Schoenoplectiella mucronata, Oplismemuis undulatifolius, Scleria gaertneri, Cenchrus purpureus, Aeginetia spp. L., Lycium ferocissimum Miers, Digitaria abyssinica (A. Rich) Stapf, Pennisetum macrourum Trinius, Mikania cordata (Burm. f.) B. L. Robins, Prosopis alpataco R. A. Philippi, Prosopis sericantha Gillies ex Hook. & Arn., Alectra spp. Thunb., Drymaria arenarioides Humboldt & Bonpland, Prosopis argentina Burkart, Prosopis humilis Gillies ex Hooker Arnott, Prosopis elata (Burkart) Burkart, Prosopis campestris Griesbach, Prosopis hassleri Harms ex Hassler, Prosopis denudans Bentham, Eichhornia azurea (Swartz) Kunth, Moraea ochroleuca (Salisb.) Drapiez, Prosopis strombulifera (Lamarck) Bentham, Sagittaria sagittifolia Linnaeus, Monochoria hastata (L.) Solms, Melastoma malabathricum L., Prosopis kuntzei Harms ex Hassler, Commelina benghalensis L., Prosopis articulata S. Watson, Sparganium erectum L., Imperata brasiliensis Trinius, Inula britannica L., Spermacoce alata Aubl., Orobanche spp. (nonnative) L., Oryza rufipogon Griffiths, Prosopis caldenia Burkart, Moraea collina Thunb., Moraea pallida (Baker) Goldblatt, Arctotheca calendula (L.) Levyns, Mimosa pigra L., Setaria pumila ssp. pallidefusca (Schumacher) B. K. Simon, Leptochloa chinensis (L.) Nees, Prosopis ferox Griesbach, Lygodium flexuosum (L.) Sw. (1801) (Mobot), Tridax procumbens L., Imperata cylindrica (L.) Beauv., Crupina vulgaris Cass., Ageratina riparia (Regel) King & H. E. Robins., Ageratina adenophora (Spreng.) King & H. E. Robins., Prosopis calingastana Burkart, Cuscuta (non-native) L., Ottelia alismoides (Linnaeus) Pers., Salvinia auriculata Aublet, Acacia nilotica (L.) Willd. ex Delile, Rubus fruticosus L., Azolla pinnata R. Brown, Euphorbia terracina L., Heracleum mantegazzianum Sommier & Levier, Salvinia molesta D. S. Mitchell, Mimosa diplotricha C. Wright ex Sauvalle, Galega officinalis L., Salvinia herzogii de la Sota, Onopordum acaulon L., Hydrilla verticillata (L. f.) Royle, Onopordum illyricum L., Rottboellia cochinchinensis (Lour.) W. D. Clayton, Opuntia aurantiaca Lindley, Prosopis pallida (Humb. & Bonpl. ex Willd.) Kunth, Pennisetum clandestinum Hochst. ex Chiov., Pennisetum pedicellatum Trinius, Prosopis torquata (Cavan. ex Lagasca y Segura) sessiliflora (Vahl) Blume, Urochloa DC., Limnophila panicoides Beauvois, Salvinia biloba Raddi, Oryza longistaminata A. Chev. & Roehr., Senecio madagascariensis Poir., Caulerpa taxifolia (Vahl) C. Agardth, Salsola vermiculata L., Melaleuca quinquenervia (Cav.) Blake, Prosopis ruizlealii Burkart, Prosopis burkartii Muñoz, Prosopis fiebrigii Harms, Prosopis rojasiana Burkart, Prosopis castellanosii Burkart, Mikania micrantha Kunth, Hygrophila polysperma (Roxb.) T. Anders., Pennisetum polystachion (Linnaeus) Schultes, Rubus moluccamuis L., Monochoria vaginalis (Burm. f.) K. Presl ex Kunth, Lygodium microphyllum (Cav.) R. Br., Moraea flaccida (Sweet) Steud., Asphodelus fistulosus Linnaeus, Lagarosiphon major (Ridley) Moss, Prosopis palmeri S. Watson, Chrysopogon aciculatus (Retz.) Trin., Oryza punctata Kotzchy ex Steud., Paspalum scrobiculatum Linnaeus, Ischaemum rugosum Salisbury, Nassella trichotoma Hackel ex Arech., Alternanthera sessilis (L.) R. Br. ex DC., Senecio inaequidens DC., Emex spinosa (Linnaeus) Campdera, Avena sterilis Linnaeus, Ipomoea aquatica Forssk., Prosopis farcta (Banks & Soland.) J. F. Macbr., Emex australis Steinhall, Prosopis reptans Benth., Solanum viarum Dunal, Solanum torvum Sw., Moraea miniata Andrews, Digitaria velutina (Forsk.) Beauv., Prosopis ruscifolia Griesbach, Solanum tampicense Dunal, Carthamus oxyacantha M. Bieberstein, Saccharum spontaneum and L., Striga Lour.

In some embodiments, a weed or invasive species can be controlled by either population control—by driving down the population as described herein or by expressing one or more cargo genes that makes them susceptible to herbicide or environmental factors that lead to population control or population suppression.

In some embodiments, a method for gene drive-mediated population modification of an organism that expresses one or more cargo gene(s) is provided. In some embodiments, the cargo gene encodes for a protein that makes the organism resistant to an external stimulus. In some embodiments, the cargo gene encodes for a protein that makes the organism susceptible to an external stimulus. Some non-limiting examples of external stimuli include temperature, stress, drought, inadequate soil fertility, nutrient deficiencies, pollution, salinity, light, water levels, presence of a weed or pest, competitor or pathogen.

In some embodiments, a method for gene drive-mediated population suppression of an organism is provided. In some embodiments, one or more vectors are introduced into an individual organism of the population. In some embodiments, the organism is a plant. In some embodiments, the vector comprises a first gene encoding a rescue transgene; a first enhancer/promoter operably linked to a second gene encoding the DNA sequence modifying enzyme and optionally, one or more regulatory sequences; a second gene encoding a DNA sequence modifying enzyme; and a second promoter operably linked to one or more guide RNAs (gRNAs) targeting the endogenous copy of an essential gene; and one or more guide RNAs (gRNAs) targeting the endogenous copy of an essential gene. In some embodiments, the vector comprises one or more cargo sequences. In some embodiments, the DNA modifying enzyme modifies an endogenous copy of a gene essential for diploid fertility of one sex. In some embodiments, the one or more vectors is inserted into a gene required for diploid fertility of one sex, thereby disrupting the gene required for diploid fertility of one sex. In some embodiments, the method comprises expressing the DNA sequence modifying enzyme in a cell of the organism that ultimately gives rise to a germline. The expression of the DNA sequence modifying enzyme induces one or more sequence modifications in the cell, such that the one or more sequence modifications result in the essential gene being rendered partially or wholly non-functional, resulting in death or loss of ability to participate in fertilization if the cell lacks the rescue transgene. In some embodiments, the partially or wholly non-functional gamete is rescued by the rescue transgene, generating an altered gamete containing one or more vectors. In some embodiments, the altered gamete in which the essential gene has been rendered partially or wholly non-functional has been rescued by the rescue transgene. In some embodiments, the method comprises introducing a progeny carrying the altered gamete containing one or more vectors in an environment wherein an increase in the frequency of the rescue transgene bearing progeny is desired relative to a frequency of a wild organism in a population of the organism. In some embodiments, killing of gametes that fail to inherit the vector results in a relative increase in the frequency of those that do carry it. In some embodiments, the increase in frequency of the transgene-bearing progeny results in the creation of diploids who are sterile in one sex due to the presence of two copies of the vector, which disrupt both copies of the gene essential for diploid fertility of that sex.

In some embodiments, the one or more vectors for population suppression is inserted on a gene that is required for diploid fertility of one sex. In females the gene may be, for example, Bell1. In males the gene may be, for example, opr3, ms1, ms2, non-dehiscence1, delayed-dehiscence1, or POLLENLESS3. In some embodiments, the modification where one or more vectors for mediating population suppression are located on a gene required for diploid fertility of one sex, the gametes are homozygous for ClvR and not heterozygous. In this configuration heterozygotes of both sexes for the vector are fertile, while homozygotes for one sex are sterile. Since only vector-bearing gametes can survive, the only survivors are homozygous for the vector. Homozygotes for one of the sexes are sterile. Homozygotes for the other are fertile and keep spreading the vector. Eventually this results in a population crash when fertile members of one sex are reduced or even no longer present. In some embodiments, the gene is one that is required for diploid fertility of male sex. In some embodiments, the gene is one that is required for diploid fertility of female sex.

In some embodiments, one or more vectors for population suppression is inserted in a gene that is required for survival of haploid gametes of particular sex. In females these include rh32, rh29, atrh36, slow walker3. In males these include bcp1, AtMago, PIRL1 and PIRL9.

In some embodiments, the method is mediated by inserting the vector elements within the gene whose expression is required for survival of haploid gametes of particular sex. In some embodiments, the method achieves sex-biased drive that gives rise to a particular sex bearing gametes while the other sex is eliminated upon spread. In this implementation, the presence of the vector near the male-determining locus means that all surviving gametes produced by a male will carry the male-determining locus (the Y chromosome) and the vector. These males will produce more males and so on, never producing female progeny. Eventually the population runs out of females and goes extinct. In some embodiments, the gene is required for survival of female haploid gametes. In some embodiments, the gene is required for survival of male haploid gametes.

In some embodiments, the one or more vectors for population suppression is provided. In some embodiments, the method comprises obtaining the organism. In some embodiments, the method comprises: introducing into the organism one or more vectors comprising a nucleic acid sequence encoding: a first gene encoding a rescue transgene; a first enhancer/promoter operably linked to a second gene encoding the DNA sequence modifying enzyme and optionally, one or more regulatory sequences; a second gene encoding a DNA sequence modifying enzyme, wherein the DNA modifying enzyme modifies an endogenous copy of an essential gene; and a second promoter operably linked to one or more guide RNAs (gRNAs) targeting the endogenous copy of an essential gene; and one or more guide RNAs (gRNAs) targeting the endogenous copy of an essential gene, and optionally, one or more cargo sequences, wherein the one or more vectors is inserted next to or within (but not disrupting) a locus whose presence is sufficient for adult male or female sex determination, expressing the DNA sequence modifying enzyme in a cell of the organism that ultimately gives rise to a germline, inducing one or more sequence modifications in the cell, such that the one or more sequence modifications result in the essential gene being rendered partially or wholly non-functional, resulting in death or loss of ability to participate in fertilization if the cell lacks the rescue transgene, rescuing the partially or wholly non-functional gamete, by the rescue transgene, generating an altered gamete containing one or more vectors, wherein the altered gamete in which the essential gene has been rendered partially or wholly non-functional has been rescued by the rescue transgene and introducing a progeny carrying the altered gamete containing one or more vectors in an environment wherein an increase in a frequency of the rescue transgene bearing progeny is desired relative to a frequency of a wild organism in a population of the organism. In some embodiments, the vector is linked to a male determining locus, male vector-bearing adults give rise to pollen that, because it carries the male-determining gene(s), only gives rise to male, but not female, progeny. In some embodiments, this results in an increase in the frequency of males versus females, generation by generation until females are eliminated and the population collapses. In some embodiments, if the vector is linked to a female determining locus, female vector-bearing adults give rise to ovules that, because they carry the female-determining gene(s), only gives rise to female, but not male, progeny. In some embodiments, this results in an increase in the frequency of females versus males' generation by generation until males are eliminated and the population collapses.

In some embodiments, the sex-determining locus is located in the Y chromosome. In some embodiments, the sex-determining locus is located in an autosome. In some embodiments, the sex-determining locus is located in the W chromosome.

In some embodiments, the DNA sequence modifying enzyme is expressed in the organism. In some embodiments, the organism is unicellular or multicellular. In some embodiments, when the organism is multicellular, the DNA sequence modifying enzyme is expressed in all cells of the organism. In some embodiments, the DNA sequence modifying enzyme is not expressed in all cells of the multicellular organism. In some embodiments, the DNA sequence modifying enzyme is expressed in a fraction of cells of the multicellular organism. In some embodiments, the DNA sequence modifying enzyme is expressed only in the male or female germline, or in the germline of both sexes.

In some embodiments, the organism is a diploid that produces haploid gametes. In some embodiments, the diploid undergoes meiosis to form haploid gametes before undergoing fertilization to form a new diploid. In some embodiments, the progeny is formed by mating of haploid gametes. In some embodiments, the progeny is formed by mating of haploid gametes of the same species. In some embodiments, the progeny is formed by mating of haploid gametes of two related species.

In some embodiments, the organism is a polyploid that gives rise to gametes with half the number of each chromosome.

In some embodiments, the organism is a plant.

In some embodiments, the expression of the DNA sequence modifying enzyme induces one or more sequence modifications. In some embodiments, the expression of the DNA sequence modifying enzyme induces one or more sequence modifications in an essential gene. In some embodiments, the expression of the DNA sequence modifying enzyme induces one or more sequence modifications in an essential gene in one or more cells in the organism. In some embodiments, the one or more sequence modifications result in the essential gene being rendered partially non-functional. In some embodiments, the one or more sequence modifications result in the essential gene being rendered wholly non-functional. In some embodiments, the one or more sequence modifications result in the essential gene being rendered partially non-functional in some circumstances and wholly non-functional in other circumstances. In some embodiments, the result of the essential gene being rendered partially or wholly non-functional is a defect in the organism. In some embodiments, the defect is, without limitations, a defect in survival, growth control, fertility, differentiation, or combinations thereof.

In some embodiments, the defect occurs when the one or more cells in which the essential gene being rendered partially or wholly non-functional lack a rescue transgene. In some embodiments, the rescue transgene expresses a recoded protein that rescues the defects in survival, growth control, differentiation, or combinations thereof.

In some embodiments, the expression of the recoded protein by the rescue transgene results in the generations of an altered organism. In some embodiments, the altered organism expresses the recoded protein in the one or more cells in which the essential gene has been rendered at least partially non-functional. In some embodiments, the altered organism expresses the recoded protein in the one or more cells in which the essential gene has been rendered wholly non-functional. In some embodiments, the altered organism expresses the recoded protein in the one or more cells in which the essential gene has been rendered partially non-functional in some circumstances and wholly non-functional in other circumstances thereby resulting in survival of cells comprising the recoded protein.

In some embodiments, the altered organism carries one or more copies of the vector or vectors, and wherein the defects in survival, growth control, or differentiation of the one or more cells in which the essential gene has been rendered partially non-functional have been rescued by expression of the rescue transgene from the one or more copies of the vector or vectors. In some embodiments, the altered organism carries one or more copies of the vector or vectors, and wherein the defects in survival, growth control, or differentiation of the one or more cells in which the essential gene has been rendered wholly non-functional have been rescued the rescue transgene expressed from the one or more copies of the vector or vectors. In some embodiments, the altered organism carries one or more copies of the vector or vectors, and wherein the defects in survival, growth control, or differentiation of the one or more cells in which the essential gene has been rendered partially non-functional in some circumstances and wholly non-functional in other circumstances have been rescued the rescue transgene expressed from the one or more copies of the vector or vectors.

In some embodiments, the altered organism is introduced in a population. In some embodiments, the altered organism is introduced in a population in which an increase in a frequency of the altered organism is desired relative to a frequency of a wild type organism. In some embodiments, the altered organism is introduced in a population in a particular environment. In some embodiments, the altered organism is introduced in a population in a particular environment in which an increase in a frequency of the altered organism is desired relative to a frequency of a wild type organism in the population in the particular environment. In some embodiments, the altered organisms is introduced in the population such that the percent of the altered organism in the population ranges from about 0.0001% to about 50%. In some embodiments, the percent is about 0.00001, 0.0005, 0.0001, 0.0005, 0.001, 0.005, 0.01, 0.05, 0.1, 0.5, 1, 5, 10, 20, 30, 40, or 50%. In some embodiments, the percent is greater than 100%, so as to achieve a more rapid change in the population.

In some embodiments, introducing the altered organism in the population results in replacement of the wild type organism with the altered organism in the population. In some embodiments, introducing the altered organism in the population results in replacement of the wild type organism with the altered organism in the population in the particular environment.

In some embodiments, the altered organism exhibits one or more altered traits. In some embodiments, the altered organism introduces and spreads one or more traits of interest in the population. In some embodiments, the one or more traits of interest is a novel trait not previously prevalent in the population, a trait that is a modified version of a trait previously present in the population (e.g., an enhance or a suppressed version of a trait previously present in the population) or a combination thereof. In some embodiments, the population is modified by the introduction of the altered organism in the population. In some embodiments, the population is modified by the introduction of the altered organism in the population in the particular environment. In non-limiting examples of traits of interest to enhance or decrease include but are not limited to pathogen resistance, insecticide resistance, environmentally triggered death or sterility, herbicide resistance, fungicide resistance, phage resistance, resistance to viral infection, resistance to abiotic environmental factors, such as cold, heat, stress and resistance to competitors.

In some embodiments, the population is modified by the introduction of the altered organism in the population. In some embodiments, the population is modified by the introduction of the altered organism in the population in the particular environment. Non-limiting examples of traits of interest include but are not limited to pathogen resistance, insecticide resistance, environmentally triggered death or sterility, herbicide resistance, fungicide resistance, phage resistance, resistance to viral infection, resistance to abiotic environmental factors, such as cold, heat, and stress. In some embodiments the population is modified such that gene drive is limited in time and space by segregation of the vectors that make up versions of two locus ClvR.

In some embodiments, the population is modified by the introduction of the altered organism in the population. In some embodiments, the population is modified by the introduction of the altered organism in the population in the particular environment. In non-limiting examples of traits of interest include but are not limited to pathogen resistance, insecticide resistance, environmentally triggered death or sterility, herbicide resistance, fungicide resistance, phage resistance, resistance to viral infection, resistance to abiotic environmental factors, such as cold, heat, and stress. In some embodiments the modified population is eliminated or greatly decreased with respect to one or more component transgenes through dilution of the population with wildtypes.

In some embodiments, a method of reversibly modifying a population is provided. In some embodiments, the method comprises obtaining a wild type organism, positioning a two-vector system in the wild type organism generating an altered organism by inducing one or more sequence modifications in an essential gene by a DNA sequence modifying enzyme/complex in the two-vector system that result in a defect in survival, growth control, fertility, or differentiation in one or more cells in the organism, and rescuing the defect in survival, growth control, fertility, or differentiation by a rescue transgene in the two-vector system, introducing the altered organism in an environment wherein an increase in a frequency of the altered organism is desired relative to a frequency of the wild type organism in a population; replacing the wild type organism with the altered organism in the population in the environment, thereby obtaining a modified population. In some embodiments one can then reintroduce the wild type organism in an environment wherein an increase in a frequency of the wild type organism is desired relative to a frequency of the modified organism in the modified population. This will result in replacing the modified organism with the wild type organism in the modified population in the environment, thereby reversibly modifying the population.

In some embodiments of the method, the one or more cells comprise somatic cells, germline cells, gametes, or a combination thereof.

In some embodiments of the method, the altered organism is heterozygous or homozygous for one or both of the vectors.

In some embodiments of the method, the organism is haploid, diploid, or polyploid.

In some embodiments of the method, the reversible modification of the population occurs at a rapid rate, high frequency, or both. In some embodiments of the method, the rapid rate is defined as replacement of at least 90% of the wild type organism by the altered organism or vice versa in the population after at most 100 generations. In some embodiments of the method, the high frequency is defined as replacement of at least 90% of the wild type organism by the altered organism or vice versa after 100 generations in the population.

In some embodiments, an organism with the defect in survival, growth control, fertility, or differentiation of the one or more cells is eliminated if the one or more cells of the organism lack the rescue transgene.

In some embodiments, the DNA sequence modifying enzyme does not modify the rescue transgene.

In some embodiments, rescuing the defects in one or more of survival, growth control, or differentiation is achieved by restoring one or more of normal survival, growth control, fertility, or differentiation of the one or more cells by the rescue transgene.

In some embodiments, the one or more cells comprise somatic cells, germline cells, gametes, or a combination thereof.

In some embodiments, the DNA sequence modifying enzyme is a nuclease, a base editor, transposase, or a Search and Replace Prime editor according to the embodiments herein.

In some embodiments, the nuclease cleaves and generates one or more double strand breaks in the endogenous copy of the essential gene as described herein.

In some embodiments, the one or more double strand breaks are repaired to create an altered sequence comprising insertions, deletions, base alterations, or a combination thereof.

In some embodiments, the base editor creates one or more base changes or small insertions/deletions in the endogenous copy of the essential gene.

In some embodiments, the one or more base changes comprise one or more point mutations, or deamidated bases that are replaced with nucleotides of a different sequence.

In some embodiments the Search and Replace Prime editor creates one or more base changes or insertion/deletions in the endogenous copy of the essential gene.

In some embodiments, the altered organism is heterozygous or homozygous for the vector or vectors.

In some embodiments, the organism is haploid. Non-limiting example of haploid organisms include prokaryotes. In some embodiments, the organism is diploid. Non-limiting example of diploid organisms include insects, fungi, many plants and animals. In some embodiments, the organism is polyploidy. Non-limiting examples of polyploid organisms include some fungi and animals and many plants.

In some embodiments, the organism is selected from the group consisting of prokaryotes, fungi, plants, and animals. In some embodiments, the organism is, without limitations, a prokaryote (bacteria, archaea), fungi, insect, mammal, rodent, fish, amphibian, reptile or plant. In some embodiments the organism is a plant.

In some embodiments, the plant is a weed or an invasive species. In some embodiments, the weed or invasive species include, but are not limited to Imperata cylindrica, Arundo donax, Cyperus esculentus, Festuca arundinacea, Microstegium vimineum, Miscanthus sinensis, Panicum repens, Phragmites australis, Phyllostachys aurea, Pseudosasa japonica, Sorghum halepense, Bambusa, Dactylis glomerata, Pennisetum clandestinum, Oryza punctata, Oryza longistaminata, Leptochloa chinensis, Nassella trichotoma, Setaria pumila ssp. pallidefusca, Saccharum spontaneum, Pennisetum macrourum, Paspalum scrobiculatum, Rottboellia cochinchinensis, Ischaemum rugosum, Chrysopogon aciculatus, Digitaria velutina, Oryza rufipogon, Pennisetum pedicellatum, Pennisetum polystachion, Avena sterilis, Digitaria abyssinica, Imperata brasiliensis, Urochloa panicoides, Achnatherum brachychaetum, Aegilops cylindrica, Aegilops geniculata, Aegilops triuncialis, Agrostis stolonifera, Alopecurus myosuroides, Ammophila arenaria, Andropogon virginicus, Arrhenatherum elatius, Arthraxon hispidus, Avena barbata, Avena fatua, Axonopus fissifolius, Bromus arvensis, Bromus catharticus, Bromus racemosus, Brachypodium distachyon, Bromus diandrus, Bromus hordeaceus, Bromus hordeaceus ssp. hordeaceus, Bromus inermis, Bromus rubens, Bromus secalinus, Bromus sterilis, Bromus tectorum, Cenchrus echinatus, Cenchrus longispinus, Cenchrus spinifex, Cortaderia jubata, Cortaderia selloana, Cynodon dactylon, Cyperus difformis, Cyperus haspan, Cyperus involucratus, Cyperus iria, Cyperus prolifer, Cyperus rotundus, Dactyloctenium aegyptium, Digitaria ciliaris, Digitaria sanguinalis, Echinochloa crus-galli, Ehrharta calycina, Ehrharta erecta, Microlaena stipoides, Eleusine indica, Elymus repens, Eragrostis cilianensis, Eriochloa villosa, Heteropogon contortus, Hordeum jubatum, Holcus lanatus, Hordeum murinum ssp. leporimim, Hymenachne amplexicaulis, Hyparrhenia rufa, Imperata brevifolia, Juncus effusus, Juncus planifolius, Lolium perenne, Festuca pratensis, Melinis minutiflora, Melinis repens, Milium vernale, Muhlenbergia schreberi, Nardus stricta, Neyraudia reynaudiana, Panicum antidotale, Panicum capillare, Paspalum conjugatum, Paspalum dilatatum, Panicum notatum, Paspalum miliaceum, Paspalum urvillei, Panicum virgatum, Pennisetum glaucum, Cenchrus setaceus, Phalaris aquatica, Phalaris arundinacea, Phalaris canariensis, Phalaris minor, Phyllostachys nigra, Phleum pratense, Poa annua, Poa bulbosa, Poa compressa, Polypogon monspeliensis, Poa pratensis, Poa trivialis, Rhynchospora caduca, Sacciolepis indica, Schismus arabicus, Schismus barbatus, Schizachyrium condensatum, Secale cereale, Setaria faberi, Setaria italica, Setaria parviflora, Setaria palmifolia, Setaria pumila, Setaria verticillata, Setaria viridis, Setaria viridis var. major, Setaria viridis var. viridis, Sorghum×almum, Sorghum bicolor, Sorghum bicolor ssp. drummondii, Spartina alterniflora, Spartina anglica, Spartina densiflora, Sporobolus indicus, Spartina patens, Taeniatherum caput-medusae, Megathyrsus maximus, Urochloa mutica, Urochloa texana, Ventenata dubia, Vossia cuspidata, Vulpia myuros, Eragrostis curvula, Agrostis spp., Agrostis gigantea, Carex kobomugi, Agrostis capillaris, Lolium temulentum, Pennisetum spp., Lolium spp., Cyperus entrerianus, Anthoxanthum odoratum, Cyperus brevifolioides, Bambusa vulgaris, Hemarthria altissima, Poa nemoralis, Hordeum murinum ssp. glaucum, Miscanthus sacchariflorus, Kyllinga brevifolia, Aira caryophyllea, Luzula luzuloides, Carex longii, Carex flaccosperma, Kyllinga nemoralis, Zizaniopsis miliacea, Briza maxima, Apera interrupta, Axonopus compressus, Brachypodium sylvaticum, Bromus briziformis, Chloris barbata, Coix lacryma-jobi, Cyperus microiria, Digitaria eriantha, Digitaria insularis, Digitaria setigera, Digitaria violascens, Echinochloa colona, Eragrostis cumingii, Eragrostis lehmanniana, Eriochloa fatmensis, Festuca trachyphylla, Festuca rubra, Vulpia bromoides, Thinopyrum ponticum, Thinopyrum intermedium, Stenotaphrum secundatum, Sporobolus indicus var. capensis, Sporobolus diandrus, Garnotia stricta, Gastridium phleoides, Holcus mollis, Hordeum murinum, Juncus ensifolius, Rytidosperma biannulare, Rytidosperma penicillatum, Polypogon viridis, Oplismenus hirtellus, Paspalum fimbriatum, Paspalum quadrifarium, Scleria lacustris, Cyperus spp., Sorghum, Setaria spp., Eremochloa ophiuroides, Paspalum vaginatum, Oxycaryum cubense, Agropyron desertorum, Agrostis avenacea, Alopecurus pratensis, Bothriochloa bladhii, Carex acutiformis, Cynosurus echinatus, Dichanthium annulatum, Dichanthium aristatum, Ehrharta longiflora, Glyceria declinata, Nassella tenuissima, Pennisetum villosum, Phyllostachys spp., Bromus diandrus var. rigidus, Lolium multiflorum, Glyceria maxima, Bambusa multiplex, Digitaria spp., Urochloa distachya, Alopecurus geniculatus, Poa pratensis ssp. pratensis, Leymus arenarius, Bromus japonicus, Pseudosasa, Tribolium obliterum, Scleria eggersiana, Juncus inflexus, Festuca filiformis, Carex pendula, Carex flacca, Phyllostachys bambusoides, Carex macrocephala, Briza minor, Dactylis glomerata ssp. glomerata, Arundinaria pygmaea, Phragmites spp., Thinopyrum junceiforme, Hordeum marinum, Miscanthus, Bromus inermis ssp. inermis var. inermis, Bromus inermis ssp. inermis, Anthoxanthum odoratum ssp. odoratum, Arrhenatherum elatius var. elatius, Hyparrhenia hirta, Nassella manicata, Scleria microcarpa, Carex disticha, Agrostis canina, Puccinellia distans, Sporobolus indicus var. pyramidalis, Bothriochloa ischaemum, Setaria pumila ssp. pumila, Phyllostachys aureosulcata, Typha laxmannii, Spartina foliosa×alterniflora, Luziola subintegra, Saccharum ravennae, Phragmites australis ssp. australis, Setaria glauca, Tripidium ravennae ssp. ravennae, Bambusa bambos, Miscanthus giganteus, Stipa capensis, Cenchrus ciliaris, Cenchrus polystachios, Rytidosperma caespitosum, Stipa miliacea var. miliacea, Pleioblastus fortunei, Dianella ensifolia, Arthraxon hispidus var. hispidus, Brachypodium sylvaticum ssp. sylvaticum, Cenchrus alopecuroides, Schoenoplectiella mucronata, Oplismenus undulatifolius, Scleria gaertneri, Cenchrus purpureus, Aeginetia spp. L., Lycium ferocissimum Miers, Digitaria abyssinica (A. Rich) Stapf, Pennisetum macrourum Trinius, Mikania cordata (Burm. f.) B. L. Robins., Prosopis alpataco R. A. Philippi, Prosopis sericantha Gillies ex Hook. & Arn., Alectra spp. Thunb., Drymaria arenarioides Humboldt & Bonpland, Prosopis argentina Burkart, Prosopis humilis Gillies ex Hooker & Arnott, Prosopis elata (Burkart) Burkart, Prosopis campestris Griesbach, Prosopis hassleri Harms ex Hassler, Prosopis denudans Bentham, Eichhornia azurea (Swartz) Kunth, Moraea ochroleuca (Salisb.) Drapiez, Prosopis strombulifera (Lamarck) Bentham, Sagittaria sagittifolia Linnaeus, Monochoria hastata (L.) Solms, Melastoma malabathricum L., Prosopis kuntzei Harms ex Hassler, Commelina benghalensis L., Prosopis articulata S. Watson, Sparganium erectum L., Imperata brasiliensis Trinius, Inula britannica L., Spermacoce alata Aubl., Orobanche spp. (nonnative) L., Oryza rufipogon Griffiths, Prosopis caldenia Burkart, Moraea collina Thunb., Moraea pallida (Baker) Goldblatt, Arctotheca calendula (L.) Levyns, Mimosa pigra L., Setaria pumila ssp. pallidefusca (Schumacher) B. K. Simon, Leptochloa chinensis (L.) Nees, Prosopis ferox Griesbach, Lygodium flexuosum (L.) Sw. (1801) (Mobot), Tridax procumbens L., Imperata cylindrica (L.) Beauv., Crupina vulgaris Cass., Ageratina riparia (Regel) King & H. E. Robins., Ageratina adenophora (Spreng.) King & H. E. Robins., Prosopis calingastana Burkart, Cuscuta (non-native) L., Ottelia alismoides (Linnaeus) Pers., Salvinia auriculata Aublet, Acacia nilotica (L.) Willd. ex Delile, Rubus fruticosus L., Azolla pinnata R. Brown, Euphorbia terracina L., Heracleum mantegazzianum Sommier & Levier, Salvinia molesta D. S. Mitchell, Mimosa diplotricha C. Wright ex Sauvalle, Galega officinalis L., Salvinia herzogii de la Sota, Onopordum acaulon L., Hydrilla verticillata (L. f.) Royle, Onopordum illyricum L., Rottboellia cochinchinensis (Lour.) W. D. Clayton, Opuntia aurantiaca Lindley, Prosopis pallida (Humb. Bonpl. ex Willd.) Kunth, Pennisetum clandestinum Hochst. ex Chiov., Pennisetum pedicellatum Trinius, Prosopis torquata (Cavan. ex Lagasca y Segura) DC., Limnophila sessiliflora (Vahl) Blume, Urochloa panicoides Beauvois, Salvinia biloba Raddi, Oryza longistaminata A. Chev. & Roehr., Senecio madagascariensis Poir., Caulerpa taxifolia (Vahl) C. Agardth, Salsola vermiculata L., Melaleuca quinquenervia (Cav.) Blake, Prosopis ruizlealii Burkart, Prosopis burkartii Muñoz, Prosopis fiebrigii Harms, Prosopis rojasiana Burkart, Prosopis castellanosii Burkart, Mikania micrantha Kunth, Hygrophila polysperma (Roxb.) T. Anders., Pennisetum polystachion (Linnaeus) Schultes, Rubus moluccanus L., Monochoria vaginalis (Burm. f.) K. Presl ex Kunth, Lygodium microphyllum (Cav.) R. Br., Moraea flaccida (Sweet) Steud., Asphodelus fistulosus Linnaeus, Lagarosiphon major (Ridley) Moss, Prosopis palmeri S. Watson, Chrysopogon aciculatus (Retz.) Trin., Oryza punctata Kotzchy ex Steud., Paspalum scrobiculatum Linnaeus, Ischaemum rugosum Salisbury, Nassella trichotoma Hackel ex Arech., Alternanthera sessilis (L.) R. Br. ex DC., Senecio inaequidens DC., Emex spinosa (Linnaeus) Campdera, Avena sterilis Linnaeus, Ipomoea aquatica Forssk., Prosopis farcta (Banks & Soland.) J. F. Macbr., Emex australis Steinhall, Prosopis reptans Benth., Solanum viarum Dunal, Solanum torvum Sw., Moraea miniata Andrews, Digitaria velutina (Forsk.) Beauv., Prosopis ruscifolia Griesbach, Solamim tampicense Dunal, Carthamus oxyacantha M. Bieberstein, Saccharum spontaneum and L., Striga Lour.

In some embodiments, the organism may be an insect. An insect can be a direct pest or indirect pest. A “direct pest” refers to insects that can cause damage at one or more stage of their life cycle by, for example, eating crops or damaging animals. The New World screw-worm fly Cochliomyia hominivorax, for example, is a direct pest of cattle, and the spotted wing Drosophila, Drosophila suzukii is pest of many fruit crops. An “indirect pest” refers to insects that transmit human diseases, for example, mosquitoes which carry malaria. Indirect pests of organisms other than humans, such as livestock or plants are also known.

In some embodiments, the insect that transmits human disease are either agricultural pests or pests of wild species used in forestry. In some embodiments, the insects are wild insect populations.

In some embodiments, the population has about 10,000 to about 100,000,000,000 organisms. In some embodiments, the population has about 500, 1000, 1500, 2000, 2500, 3000, 3500, 4000, 4500, 5000, 5500, 6000, 6500, 7000, 7500, 8000, 8500, 9000, 9500, 10,000, 100,000, 500,000, 1,000,000, 100,000,000, 1,000,000,000, 100,000,000,000 or 1,000,000,000,000 organisms, or a number within a range defined by any two of the aforementioned values.

In some embodiments, the environment comprises an open environment, a bioreactor, or a multicellular body, a closed container, or combinations thereof. In some embodiments, the environment is a combination of an open environment, a bioreactor, or a multicellular body, a closed container, and the environment changes sequentially from one to the other

In some embodiments, the wild type organism is replaced at a high frequency with the altered organism in the population wherein the wild type organism is present. In some embodiments, the wild type organism is replaced at a high frequency with the altered organism in the population in a particular environment wherein the wild type organism is present. In some embodiments, high frequency is defined as replacement of at least 90% of the wild type organism with the altered organism after 50 generations. In some embodiments, high frequency is defined as replacement of at least 80% of the wild type organism with the altered organism after 50 generations. In some embodiments, high frequency is defined as replacement of at least 70% of the wild type organism with the altered organism after 50 generations. In some embodiments, high frequency is defined as replacement of at least 60% of the wild type organism with the altered organism after 50 generations. In some embodiments, high frequency is defined as replacement of at least 50% of the wild type organism with the altered organism after 50 generations.

In some embodiments, the wild type organism is replaced at a rapid rate with the altered organism in the population wherein the wild type organism is present. In some embodiments, the wild type organism is replaced at a rapid rate with the altered organism in the population in a particular environment wherein the wild type organism is present. In some embodiments, rapid rate is defined as replacement of at least 90% of the wild type organism with the altered organism after 50 generations. In some embodiments, rapid rate is defined as replacement of at least 80% of the wild type organism with the altered organism after 50 generations. In some embodiments, rapid rate is defined as replacement of at least 70% of the wild type organism with the altered organism after 50 generations. In some embodiments, rapid rate is defined as replacement of at least 60% of the wild type organism with the altered organism after 50 generations. In some embodiments, rapid rate is defined as replacement of at least 50% of the wild type organism with the altered organism after 50 generations.

In some embodiments, at least 90% of the wild type organism is replaced with the altered organism after 50 generations. In some embodiments, at least 90% of the wild type organism is replaced with the altered organism after 50 generations. In some embodiments, at least 80% of the wild type organism is replaced with the altered organism after 50 generations. In some embodiments, at least 70% of the wild type organism is replaced with the altered organism after 50 generations. In some embodiments, at least 60% of the wild type organism is replaced with the altered organism after 50 generations. In some embodiments, at least 50% of the wild type organism is replaced with the altered organism after 50 generations.

In some embodiments, the one or more sequence modifications in the one or more cells is a result of the one or more cells carrying the first gene encoding the DNA sequence modifying enzyme transported through meiosis.

In some embodiments, the vector or vectors are positioned in one or more chromosomes or extra-chromosomal elements by a homologous recombination-dependent integration, random integration, integration using transposition, integration using a recombinase, or combinations thereof.

In some embodiments, the one or more cargo sequences comprise a one or more foreign gene sequences, or one or more alleles of an endogenous chromosomal or extra-chromosomal gene to which the vector has been linked through nearby insertion on the chromosome or extra-chromosomal element that carries the endogenous allele of interest.

In some embodiments, when the vector is positioned on the chromosome or the extra-chromosomal element, the first gene operably linked to the first promoter, the second gene operably linked to the second promoter, and the one or more cargo transgenes are genetically linked.

In some embodiments, when two vectors are utilized, these are positioned on distinct chromosomes or on the same chromosome at some distance with respect to each other. Each gene in the vectors may be operably linked to a separate promoter and/or other regulatory elements. If present, one or more cargo transgenes are in some cases genetically linked.

In some embodiments of the method, the vector and cargo are located in a small chromosomal inversion. In some embodiments of the method, the vector and cargo are located in a small chromosomal inversion further limiting the possibility that the vector and cargo can separate from each other during any stage of DNA replication, mitosis, and/or or meiosis.

In some embodiments, the nuclease cleaves and generates one or more double strand breaks in the endogenous copy of the essential gene with a high cleavage efficiency. In some embodiments, the high cleavage frequency is defined as at least 30% of individuals carrying the nuclease cleave the endogenous copy of the essential gene in each generation. In some embodiments, the high cleavage frequency is defined as at least 40% of individuals carrying the nuclease cleave the endogenous copy of the essential gene being cleaved in each generation. In some embodiments, the high cleavage frequency is defined as at least 50% of individuals carrying the nuclease cleave the endogenous copy of the essential gene being cleaved in each generation. In some embodiments, the high cleavage frequency is defined as at least 60%, 70%, 80%, 90% or more of individuals carrying the nuclease cleave the endogenous copy of the essential gene being cleaved in each generation.

In some embodiments, the high cleavage frequency is defined as the nuclease cleaving one or more alleles of the endogenous copy of the essential gene in at least 30% of organisms carrying the vector or vectors and the one or more alleles of the endogenous copy of the essential gene in each generation. In some embodiments, the high cleavage frequency is defined as the nuclease cleaving one or more alleles of the endogenous copy of the essential gene in at least 40% of organisms carrying the vector or vectors and the one or more alleles of the endogenous copy of the essential gene in each generation. In some embodiments, the high cleavage frequency is defined as the nuclease cleaving one or more alleles of the endogenous copy of the essential gene in at least 50% of organisms carrying the vector and the one or more alleles of the endogenous copy of the essential gene in each generation. In some embodiments, the high cleavage frequency is defined as the nuclease cleaving one or more alleles of the endogenous copy of the essential gene in at least 60% or more of organisms carrying the vector or vectors and the one or more alleles of the endogenous copy of the essential gene in each generation.

In some embodiments, the base editor creates one or more base changes in endogenous copy of the essential gene with a high base editing frequency. In some embodiments, the high base editing frequency is defined as base editing in at least 20% of organisms that carry the vector or vectors in each generation. In some embodiments, the high base editing frequency is defined as base editing in at least 30% of organisms that carry the vector in each generation. In some embodiments, the high base editing frequency is defined as base editing in at least 40% of organisms that carry the vector or vectors in each generation. In some embodiments, the high base editing frequency is defined as base editing in at least 50% or more of organisms that carry the vector or vectors in each generation.

In some embodiments, the high base editing frequency is defined as the base editor modifying one or more alleles of the endogenous copy of the essential gene in at least 20% of the organisms carrying the vector or vectors and the one or more alleles of the endogenous copy of the essential gene in each generation. In some embodiments, the high base editing frequency is defined as the base editor modifying one or more alleles of the endogenous copy of the essential gene in at least 30% of the organisms carrying the vector or vectors and the one or more alleles of the endogenous copy of the essential gene in each generation. In some embodiments, the high base editing frequency is defined as the base editor modifying one or more alleles of the endogenous copy of the essential gene in at least 40% of the organisms carrying the vector or vectors and the one or more alleles of the endogenous copy of the essential gene in each generation. In some embodiments, the high base editing frequency is defined as the base editor modifying one or more alleles of the endogenous copy of the essential gene in at least 50% or more of the organisms carrying the vector or vectors and the one or more alleles of the endogenous copy of the essential gene in each generation.

In some embodiments of the methods, the nuclease cleaves and generates one or more double strand breaks in the endogenous copy of the essential gene with a high cleavage efficiency. In some embodiments of the method, the high cleavage frequency is defined as the nuclease cleaving the endogenous copy of the essential gene in at least 30% of the organisms carrying the vector or vectors in each generation. In some embodiments of the method, the base editor creates one or more base changes in the endogenous copy of the essential gene with a high base editing frequency. In some embodiments of the method, the high base editing frequency is defined as the base editor modifying the endogenous copy of the essential gene in at least 20% of the organisms carrying the vector or vectors in each generation. In some embodiments of the method, the Search and Replace Prime editor nicks the target DNA in the endogenous copy of the essential gene, and reverse transcriptase, in conjunction with a modified gRNA, creates base changes, insertions or deletions, with high efficiency. In some embodiments of the method, the high frequency of modification is defined as modification of the endogenous copy of the essential gene at a frequency of at least 20% per gRNA of organisms carrying the vector, or progeny of these organisms, in each generation.

In some embodiments, the promoter of the gene encoding the DNA sequence modifying enzyme is a female-specific promoter such that the gene encoding the DNA sequence modifying enzyme is expressed within females only. In some embodiments, female-specific expression of the DNA sequence modifying enzyme results in the DNA-modifying enzyme being present in the female gametes of the plants. In some embodiments, when a female gamete expressing the DNA sequence modifying enzyme is fertilized by a male gamete of the plant, the DNA sequence modifying enzyme from the female gamete can modify target sequence in the paternal copy provided by the male gamete. In some embodiments, there is paternal carryover wherein male gametes contribute DNA modifying activity to female gametes, resulting in modification of the copy of the target sequence provided by the female gamete. In some embodiments, there is potential for carryover. In some embodiments, modification of an allele in a zygote is achieved even when the allele is inherited from a parent that did not carry the vector.

In some embodiments, the promoter of the gene encoding the DNA sequence modifying enzyme is a germline-specific promoter such that the gene encoding the DNA sequence modifying enzyme or an essential component of this enzyme is expressed within the germline only. In some embodiments the promoter of the second gene, which drives expression of gRNAs, results in ubiquitous gRNA expression. In some embodiments, germline-specific expression of the DNA sequence modifying enzyme and the ubiquitously expressed gRNAs results in the DNA-modifying enzyme being present in the female gametes in plants. In some embodiments, when a female gamete carrying the DNA sequence modifying enzyme deposited is fertilized by a male gamete of the plant, the DNA sequence modifying enzyme from the female gamete can modify target sequence in the male gamete. This is known as maternal carryover. In some embodiments, there is paternal carryover wherein male gamete contributes DNA modifying activity to female gamete, resulting in modification of the copy of the target sequence provided by the female gamete. In some embodiments, there is potential for carryover. In consequence, in some embodiments, modification of an allele in a zygote is achieved even when the allele is inherited from a parent that did not carry the vector.

In some embodiments, as used herein, “fitness cost” is defined as a relative reduction in the number of offspring produced by, or survival of, individuals carrying the transgenic construct, as compared with wild type individuals. In some embodiments fitness cost is defined as a relative reduction in the number of offspring produced by, or survival of, individuals not carrying the transgenic construct, as compared with those who do. In some embodiments, fitness benefit is defined as a relative increase in the number of offspring produced by, or survival of, individuals carrying the transgenic construct as compared with wild type individuals.

In some embodiments, the gene encoding the DNA sequence modifying enzyme expresses within females (the female germline or cells that contribute components to the female germline), such that the DNA-modifying enzyme and any associated cofactors such as guide RNAs, whose expression may be driven by an independent promoter, are deposited into all oocytes/eggs, including those that do not inherit one or more of the vectors, and modify target sequences in the version of the essential gene provided by the father. This represents maternal carryover of DNA sequence modifying activity.

In some embodiments, paternal carryover of the DNA modifying enzyme results in modification of the maternal copy of the essential locus in eggs, including those that do not inherit the vector or vectors.

While all of these single and two locus ClvR drive mechanisms have been considered in the context of Cas9, these drive results could apply to any endonuclease or base editor used or Search and Replace Prime editor or other method of bringing about site-specific modification of DNA, used to disrupt the function of an endogenous gene. For some embodiments, one of the biggest advantages of these drives is their adaptability to new species. This is because the primary requirements are the identification of an essential gene (thousands in each organism), a recoded or sequence unrelated version of the gene (including associated regulatory sequences) that has wildtype or close to wildtype function, and a promoter and DNA sequence-modifying enzyme capable of bringing about sequence alteration of the endogenous copy of the essential gene in the germline, or germline and early embryo cells exposed to the enzyme. In some embodiments, the rescue gene is of unrelated sequence, wherein the rescue transgene encodes a protein that is functionally equivalent to a protein encoded by the essential gene, or otherwise compensates for the loss of function of the essential gene.

### Methods of Genetic Transformation

Suitable methods for transformation of plant or other cells for use with the current disclosure are believed to include virtually any method by which DNA can be introduced into a cell, such as by direct delivery of DNA such as by PEG-mediated transformation of protoplasts, desiccation/inhibition-mediated DNA uptake, electroporation, nanoparticle mediated gene transformation, ribonucleoprotein complex mediated transformation, Agrobacterium-mediated transformation and acceleration of DNA coated particles, etc. Through the application of techniques such as these, the cells of virtually any plant species may be stably transformed, and these cells developed into genetically modified plants.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in art.

Another method for delivering transforming DNA segments to plant cells in accordance with the disclosure is microprojectile bombardment (U.S. Pat. Nos. 5,550,318; 5,538,880; 5,610,042; and PCT Application WO 94/09699; each of which is specifically incorporated herein by reference in its entirety). In this method, particles may be coated with nucleic acids and delivered into cells by a propelling force.

Another method for delivering transforming DNA segments to plant cells in accordance with the disclosure is microprojectile bombardment (U.S. Pat. Nos. 5,550,318; 5,538,880; 5,610,042; and PCT Application WO 94/09699; each of which is specifically incorporated herein by reference in its entirety). In this method, particles may be coated with nucleic acids and delivered into cells by a propelling force.

Production of Plants after Genomic Editing

In addition to direct transformation of a particular plant cell with a construct prepared according to the current disclosure, genetically modified plants may be made by crossing a plant having a selected genetic modification of the disclosure to a second plant lacking the modification. Therefore, the current disclosure not only encompasses a plant directly modified or regenerated from cells which have been modified in accordance with the current disclosure, but also the progeny of such plants.

As used herein the term “progeny” denotes the offspring of any generation of a parent plant prepared in accordance with the instant disclosure, wherein the progeny comprises a selected vector(s) comprising the DNA construct. “Crossing” a plant to provide a plant line having one or more added transgenes relative to a starting plant line, as disclosed herein, is defined as the technique that result in a coding sequence of the disclosure being introduced into a plant line by crossing a starting line with a donor plant line that comprises a first selected DNA of the disclosure. One of skill in the art will understand and adapt such methods using readily identifiable literature sources.

In some embodiments, asexual reproduction or propagation may be used to obtain a progeny plant in accordance with the disclosure. Techniques to achieve asexual propagation or reproduction may include, for example, grafting, budding, top-working, layering, runner division, cuttings, rooting, T-budding, and the like. In some embodiments, one variety into which a coding sequence has been introduced may be grafted onto the rootstock of another variety. In other embodiments, a coding sequence may be introduced into the rootstock.

### Modified Plants and Plant Parts

In some embodiments, a plant, a plant part or a plant seed comprising one or more vectors, wherein the one or more vectors that comprise: a first gene encoding a rescue transgene; a second gene encoding a DNA sequence modifying enzyme, wherein the DNA modifying enzyme modifies an endogenous copy of an essential gene; a first enhancer/promoter operably linked to the second gene encoding the DNA sequence modifying enzyme and optionally, one or more regulatory sequences; a nucleic acid encoding one or more guide RNAs (gRNAs) targeting the endogenous copy of an essential gene, and a second promoter operably linked to the nucleic acid encoding one or more guide RNAs (gRNAs) and optionally one or more cargo sequences is provided. In some embodiments, the one or more vectors is configured to be positioned in a chromosome or an extra-chromosomal element.

In some embodiments, the one or more vector(s) renders the population of the plant, plant part or plant seed vulnerable to toxicity in the presence of a chemical, compound or condition. In some embodiments, the chemical or compound is an herbicide, pesticide or environmental condition such as temperature, salinity, light or water levels. In some embodiments, the gene spread renders the population of an organism less harmful to other species in the environment

In some embodiments, the gene spread renders the population of a plant resistant to toxicity in the presence of a chemical, compound or environmental condition. In some embodiments, the gene spread results in suppression or elimination of a plant population. In some embodiments, the plant is diploid or polyploid.

### Applications

In some embodiments, the methods provided herein can be applied for modification of populations for beneficial outcomes. For example, in some embodiments, in insect-borne diseases of plants, the plant can be engineered to resist infection. The engineered plant can be used to replace wild plant population in order to achieve less transmission and less disease. Such a trait (e.g., refractoriness of the engineered plant to disease transmission) is unlikely to spread into a population in the absence of gene drive because the trait may result in a fitness cost to carriers in some other context. A gene drive solution to this problem described herein is to increase the fitness cost associated with not carrying the gene of interest through DNA sequence modification-based gene drive.

In comparison to other low threshold gene drive systems, the present DNA sequence modification-based drives are all predicted to replace wild type populations quickly while bearing substantial fitness costs, and each displays characteristics that qualify them for different scenarios.

While all of these single and two locus ClvR drive mechanisms have been discussed primarily in the context of Cas9, these drive results could apply to any endonuclease or base editor used or Search and Replace Prime editor or other method of bringing about site-specific modification of an endogenous essential gene, used to disrupt the function of the endogenous gene. For some embodiments, one of the biggest advantages of these drives is their adaptability to new species. This is because the primary requirements are the identification of an essential gene (thousands in each organism), a recoded or sequence unrelated version of the gene (including associated regulatory sequences) that has wildtype or close to wildtype function, and a promoter and DNA sequence-modifying enzyme capable of bringing about sequence alteration of the endogenous copy of the essential gene in the germline, or germline and early embryo cells exposed to the enzyme.

### Additional Embodiments

Without being limited by any particular theory, one implementation of a DNA sequence-based modification-based gene drive is as follows: a cell expresses a DNA sequence modifying enzyme that alters the sequence of an essential gene, inactivating it. Progeny that inherits the DNA sequence modifying enzyme-encoding gene also inherit a rescue copy of the wildtype gene that has been cleaved. This rescue copy is both functional and uncleavable. In this way key features required for gene drive are brought about in both single and two locus configurations.

The system disclosed herein is applicable toplants that go from a haploid to diploid phase and back to haploid through meiosis. A chromosomal copy of the DNA sequence modifying enzyme and a recoded or sequence unrelated version of the rescue will be transmitted to only some progeny during meiosis. Those haploids that fail to inherit the rescue copy of the gene will die because the DNA sequence modifying enzyme, which is transmitted through cytoplasm, will cause alteration of the wildtype copy. The wildtype copy of the gene will also have been cleaved during the diploid stage. In any case, only haploid gametes (for diploid parents) that inherit the tightly linked rescue construct will survive. This constitutes a kind of gamete killing. Most generally, the system described applies to any biological situation in which a DNA sequence modifying enzyme alters the sequence of an essential gene so as to disrupt essential functions in haploid, diploid or polyploid cells. This modification can occur in the parental cell, which can be haploid, diploid, tetraploid, or polyploid. Alternatively, the DNA sequence modifying enzyme, the transcript and/or protein for which is produced in the parental cell, can alter the sequence of the essential gene in the progeny cells in which it becomes located through cytoplasmic diffusion or active transport. The operative principle in all cases is that in the relevant cell type, or in a multicellular organism, in some fraction of these cells, all or most copies of the endogenous copies of the essential gene are altered so as to produce non-functional copies of the gene. This results in death of all cells that fail to inherit one or more copies of the rescue transgene. The DNA sequence modifying enzyme, and the rescue transgene are tightly linked and behave as a single genetic unit.

In some embodiments, the model is extrapolated to a diploid animal such as a rodent, mosquito, fish, plant, amphibian, or other organism in which spermatogenesis utilizes haploid-specific promoters to drive the expression of genes essential for spermatogenesis. In some embodiments, the DNA sequence modifying enzyme is expressed in the germline at some point. It is not critical when, or in which sex. What matters is that ultimately one will end up with post-meiotic cells that carry a copy of the rescue transgene, while their post-meiotic brothers do not. To the extent that it is true that the product of the rescue transgene, which will have all the endogenous regulatory sequences of the endogenous gene, does not diffuse into the meiotic brothers to which they are still connected by cytoplasmic bridges, those sperm will die, be resorbed, or be ejaculated in a state that is non-functional. This will result in nuclease and rescue transgene-bearing meiotic products being preferentially represented in the next generation, a form of population replacement.

In some embodiments, a rescue version of a post-meiotic expressed gene that is normally present on an autosome can be expressed from the Y chromosome along with the DNA sequence modifying enzyme. Provided the DNA sequence modifying enzyme alters the wildtype endogenous copy of the gene in the germline then only Y-bearing sperm will generate this product. This holds even if the haplo-expressed gene is on the autosome. In early generations there may be some reduced sex ratio bias if some wildtype copies are not cleaved, and depending on when in germline development the nuclease acts (before or after this generations post-meiotic expression). However, the bottom line is the same. Eventually, wildtype copies of the haplo-expressed gene are lost and the only remaining functional copies are those on the Y chromosome. This can result in sex ratio distortion if the sperm in which the gene has been inactivated are unable to carry out fertilization.

In some embodiments, the “Rescue” element of two-locus ClvR (Cargo, Rescue and gRNAs) can be created by modifying the single-locus version of ClvR^{tko} from Oberhofer et al., 2019. This can be achieved by injecting Cas9/gRNA RNP-complexes into ClvR^{tko} flies. The Cas9/gRNA RNP-complexes targeted the Cas9 reading frame of ClvR^{tko} to create mutations within and abolish Cas9 function at that site.

In some embodiments, linkage is important in terms of the ability of ClvR to spread beyond a target area. In some embodiments, by titrating the degree of linkage between the two locus components one can titrate the extent of ClvR spread in space. In some embodiments, this can be appreciated by considering first the case of completely linked loci, single locus ClvR. In this case drive is always present. However, in some embodiments, when different degrees of linkage are present the two components of the system dissociate from each other specific kinetics. The important point is that regardless of the degree of linkage, as two locus ClvR spreads in space, drive will decrease as Cas9 segregates away from the Rescue-bearing components. In some embodiments, it will segregate slowly when recombination distances are low (e.g., 12.5 m.u.), and more rapidly when recombination distances are higher. In any case other than complete linkage, in some embodiments, segregation of Cas9 from Rescue-bearing constructs will ultimately result in loss of drive. In this way, in some embodiments, any degree of linkage makes two locus ClvR ultimately a self-limiting drive system with respect to spread in space. In some embodiments, two locus Clvr can spread to genotype fixation in a constrained area in which all the wildtype copies of the essential gene have been lost (genetic addiction). But, in some embodiments, when spread in space is not constrained, the ultimate loss of Cas9 through segregation and loss in dead individuals who lack functional copies of the essential gene results in loss of drive potential.

Embodiments of the present disclosure provided herein are described further by way of the following exemplary embodiments:

1. A method for performing spread of a gene or multi-gene cassette in a population of an organism, comprising:
(i) obtaining the organism,
(ii) introducing into the organism one or more vectors comprising a nucleic acid sequence encoding:
   a. a first gene encoding a rescue transgene;
   b. a first enhancer/promoter operably linked to a second gene encoding the DNA sequence modifying enzyme and optionally, one or more regulatory sequences;
   c. a second gene encoding a DNA sequence modifying enzyme;
   d. a second promoter operably linked to one or more genes encoding a guide RNA (gRNA) targeting the endogenous copy of an essential gene; and
   e. one or more genes encoding a guide RNA (gRNA) targeting the endogenous copy of an essential gene, and
   f. optionally, one or more cargo sequences,
(iii) expressing the DNA sequence modifying enzyme in a cell or cells of the organism that ultimately gives rise to a germline such that the essential gene is rendered partially or wholly non-functional,
(iv) generating an altered gamete containing the one or more vectors, wherein the altered gamete in which the essential gene has been rendered partially or wholly non-functional has been rescued by the rescue transgene in the one or more vectors, and
(v) introducing a progeny carrying the altered gamete in an environment to generate a population of the organism, wherein progeny carrying the vector comprising the rescue gene increases in frequency relative to a frequency of a wild type individual organism in the population of the organism.

2. The method of embodiment 1, wherein the one or more vectors are configured such that the second gene, the first enhancer/promoter, the second promoter and/or the nucleic acid encoding one or more gRNAs are positioned on a different chromosome or an extra-chromosomal element than the first gene.

3. The method of embodiment 1, wherein the one or more vectors are configured such that the second gene, the first enhancer/promoter, the second promoter and/or the nucleic acid encoding one or more gRNAs are positioned on the same chromosome at a distance from the first gene.

4. The method of embodiment 3, wherein the distance is less than 50 map units.

5. The method of any of the previous embodiments, wherein the organism is a diploid that produces haploid gametes.

6. The method of embodiment 5, wherein the diploid undergoes meiosis to form haploid gametes before undergoing fertilization to form a new diploid.

7. The method of any of the previous embodiments, wherein the progeny is formed by mating of haploid gametes of the same or two related species.

8. The method of any of the previous embodiments, wherein the organism is a polyploid that gives rise to gametes with half the adult number of each chromosome type.

9. The method of any of the previous embodiments, wherein the organism is a plant.

10. The method of any of the previous embodiments, wherein the organism is a pest, weed, invasive species, or species of interest for agriculture, conservation or ecosystems services.

11. The method of any of the previous embodiments, wherein the gene spread renders the population of an organism vulnerable to toxicity in the presence of a chemical, compound or condition.

12. The method of embodiment 11, wherein the chemical or compound is an herbicide, pesticide or environmental condition such as temperature, salinity, light or water levels.

13. The method of any of the previous embodiments, wherein the gene spread renders the population of an organism less harmful to other species in the environment.

14. The method of any of the previous embodiments 1, wherein the gene spread renders the population of an organism resistant to toxicity in the presence of a chemical, compound or environmental condition.

15. The method of embodiment 14, wherein the condition is drought, stress, inadequate soil fertility, nutrient deficiencies, salinity, pollution, flooding, or presence of a pest, competitor, or pathogen.

16. A method of performing a gene drive-mediated population suppression of an organism comprising:
(i) obtaining the organism,
(ii) introducing into the organism one or more vectors comprising a nucleic acid sequence encoding:
   a. a first gene encoding a rescue transgene;
   b. a first enhancer/promoter operably linked to a second gene encoding the DNA sequence modifying enzyme and optionally, one or more regulatory sequences;
   c. a second gene encoding a DNA sequence modifying enzyme;
   d. a second promoter operably linked to one or more genes encoding a guide RNA (gRNA) targeting the endogenous copy of an essential gene; and
   g. one or more genes encoding a guide RNA (gRNA) targeting the endogenous copy of an essential gene, and
   h. optionally, one or more cargo sequences,
(iii) inserting the one or more vectors into a gene required for diploid fertility of one sex, thereby disrupting the gene required for diploid fertility of one sex,
(iv) expressing the DNA sequence modifying enzyme in a cell of the organism that ultimately gives rise to a germline, such that the essential gene is rendered partially or wholly non-functional,
(v) generating an altered gamete containing one or more vectors, wherein the altered gamete in which the essential gene has been rendered partially or wholly non-functional has been rescued by the rescue transgene in the one or more vectors, and
(vi) introducing a progeny carrying the altered gamete containing one or more vectors in a population of the organism, wherein gametes that do not contain the one or more vectors do not survive such that there is a relative increase in frequency of progeny comprising the vector in a population of the organism, and wherein those progeny are sterile due to the disruption of the gene required for diploid fertility.

17. The method of embodiment 16, wherein the essential gene is required for gamete function.

18. The method of any of the previous embodiments, wherein the organism gives rise to a haploid gamete.

19. The method of any of the previous embodiments, wherein the diploid adult undergoes meiosis to form haploid gametes before undergoing fertilization to form a new diploid.

20. The method of any of the previous embodiments, wherein the progeny is formed by mating the haploid gametes with an organism of the same species.

21. The method of any of the previous embodiments, wherein the organism is a polyploid that gives rise to gametes with half the adult number of each chromosome type.

22. The method of any of the previous embodiments, wherein the organism is a plant.

23. The method of any of the previous embodiments, wherein the gene required for diploid fertility of one sex, is a gene for diploid female fertility.

24. The method of any of the previous embodiments, wherein the gene required for diploid fertility of one sex, is a gene for diploid male fertility.

25. The method of any of the previous embodiments, wherein the gene required for polyploid fertility of one sex, is a gene required for polyploid male fertility.

26. The method of any of the previous embodiments, wherein the gene required for polyploid fertility of one sex, is a gene required for polyploid female fertility.

27. A method of performing a gene drive-mediated population suppression of an organism comprising:
(i) obtaining the organism,
(ii) introducing into the organism one or more vectors comprising a nucleic acid sequence encoding:
   a. a first gene encoding a rescue transgene;
   b. a first enhancer/promoter operably linked to a second gene encoding the DNA sequence modifying enzyme and optionally, one or more regulatory sequences;
   c. a second gene encoding a DNA sequence modifying enzyme,
   d. a second promoter operably linked to one or more genes encoding a guide RNA (gRNA) targeting the endogenous copy of an essential gene; and
   i. one or more genes encoding a guide RNA (gRNA) targeting the endogenous copy of an essential gene, and
   j. optionally, one or more cargo sequences, wherein the one or more vectors is inserted next to or within a locus whose presence is sufficient for adult male or female sex determination,
(iii) expressing the DNA sequence modifying enzyme in a cell of the organism that ultimately gives rise to a germline, inducing one or more sequence modifications in the cell, such that the one or more sequence modifications result in the essential gene being rendered partially or wholly non-functional,
(iv) generating an altered gamete containing one or more vectors, wherein the altered gamete in which the essential gene has been rendered partially or wholly non-functional has been rescued by the rescue transgene in the vector, wherein the progeny carrying the vector comprising the rescue gene survive such that they increase in frequency, and
(v) introducing a progeny carrying the altered gamete containing one or more vectors in a population of the organism.

28. The method of embodiment 27, wherein, if the vector is linked to a male determining locus, male vector-bearing adults give rise to pollen that, because it carries the male-determining gene(s), only gives rise to male, but not female, progeny.

29. The method of any of the previous embodiments, wherein, if the vector is linked to a male determining locus, this results in an increase in the frequency of males versus females, generation by generation until females are eliminated and the population collapses.

30. The method of any of the previous embodiments, wherein, if the vector is linked to a female determining locus, female vector-bearing adults give rise to ovules that, because they carry the female-determining gene(s), only gives rise to female, but not male, progeny.

31. The method of any of the previous embodiments, wherein, if the vector is linked to a female determining locus, this results in an increase in the frequency of females versus males generation by generation until males are eliminated and the population collapses.

32. The method of embodiment 27, wherein the sex-determining locus is located on the Y chromosome.

33. The method of embodiment 27, wherein the sex-determining locus is located on an autosome.

34. The method of embodiment 27, wherein the sex-determining locus is located on a W chromosome.

35. The method of any of the previous embodiments, wherein the organism gives rise to a haploid gamete.

36. The method of any of the previous embodiments, wherein the diploid adult undergoes meiosis to form haploid gametes before undergoing fertilization to form a new diploid.

37. The method of any of the previous embodiments, wherein the progeny is formed by mating the haploid gametes with an organism of the same species.

38. The method of any of the previous embodiments, wherein the organism is a polyploid that gives rise to gametes with half the adult number of each chromosome type.

39. The method of any of the previous embodiments wherein the organism is a plant.

40. The method of any one of the preceding embodiments, wherein the DNA sequence modifying enzyme is a nuclease or a base editor or a transposase.

41. The method of embodiment 40, wherein the nuclease cleaves and generates one or more double strand breaks in the endogenous copy of the essential gene.

42. The method of embodiment 41, wherein the one or more double strand breaks are repaired to create an altered sequence of the essential gene.

43. The method of embodiment 40, wherein the base editor creates one or more base changes in the endogenous copy of the essential gene to create an altered sequence of the essential gene.

44. The method of embodiment 43, wherein the one or more base changes comprise one or more point mutations in the endogenous copy of the essential gene.

45. The method of embodiment 40, wherein the transposase mediates integration of vector sequences into a target site.

46. The method of any one of the preceding embodiments, wherein the rescue transgene is a recoded copy of the essential gene, encodes a protein that is functionally equivalent to a protein encoded by the essential gene, and wherein the DNA sequence modifying enzyme does not modify the rescue transgene.

47. The method of any one of the preceding embodiments, wherein the chromosome is an autosome, X chromosome, Y chromosome, Z chromosome, W chromosome or supernumerary chromosome.

48. The method of any one of the preceding embodiments, wherein the extra-chromosomal element is a plasmid or a virus.

49. The method of any one of the preceding embodiments, wherein the one or more regulatory sequences comprise one or more foreign gene sequences, or one or more alleles of an endogenous chromosomal or extra-chromosomal gene to which the vector has been linked through nearby insertion on the chromosome or extra-chromosomal element that carries the endogenous gene of interest.

50. The method of any one of the preceding embodiments, wherein the DNA sequence modifying enzyme is selected from the group consisting of Cas9, Cas-9-related RNA-guided nucleases, ZFN, TALEN, homing endonuclease, natural site-specific nucleases, engineered site-specific nucleases, prime editing enzymes, transposases, base editing enzymes, cytidine deaminase, and adenine deaminase.

51. The method of embodiment 50, wherein the Cas9 is modified.

52. The method of embodiment 51, wherein the modified Cas9 possesses increased expression and catalytic activity relative to an unmodified Cas9.

53. The method of embodiment 51, wherein the modified Cas9 carries one or more introns and/or carries at least one mutation.

54. The method of embodiment 53, wherein the mutation is K918N.

55. The method of any one of the preceding embodiments, wherein the vector(s) further comprises one or more additional sequences, wherein the one or more additional sequences allow the one or more vectors to be positioned in the chromosome or the extra-chromosomal element.

56. The method of embodiment 55, wherein the one or more additional sequences is selected from the group consisting of transposase binding site, resistance markers, transgenesis markers, LTRs, recombinase binding site, a sequence with homology to a desired location on the chromosome or the extra-chromosomal element.

57. The method of any one of the preceding embodiments, wherein the first promoter is selected from the group consisting of a germline promoter, a male specific germline promoter, a female specific germline promoter, a cell-type specific promoter, a tissue-specific promoter, a ubiquitous promoter, a promoter activated at a specific stage of mitosis, and a promoter activated at a specific stage of meiosis.

58. The method of any one of the preceding embodiments, wherein the first enhancer or promoter is selected from the group consisting of ubiquitin-10, apetala1, agamous, DMC-1, clavata-3 and CaMV35S.

59. The method of any one of the preceding embodiments, wherein the second promoter is independent of the first promoter.

60. The method of any one of the preceding embodiments, wherein the second promoter is an U6 promoter.

61. The method of embodiment 41, wherein the double strand break is repaired by a mechanism selected from the group consisting of non-homologous end joining, microhomology-mediated end joining, and incomplete homologous recombination.

62. The method of any one of the preceding embodiments, wherein size of the one or more regulatory sequences ranges from about 0.5 kb to about 500 kb.

63. The method of embodiment 40, wherein the nuclease comprises at least one nuclease domain and one or more DNA binding domains.

64. The method of embodiment 63, wherein when the DNA binding domain is a TALE or a zinc finger, the nuclease domain is provided as a single active nuclease domain, such as single chain variants of Fok1.

65. A plant, a plant part or a plant seed comprising one or more vectors, wherein the one or more vectors comprise:
a first gene encoding a rescue transgene;
a second gene encoding a DNA sequence modifying enzyme, wherein the DNA modifying enzyme modifies an endogenous copy of an essential gene;
a first enhancer/promoter operably linked to the second gene encoding the DNA sequence modifying enzyme and optionally, one or more regulatory sequences;
a nucleic acid encoding one or more guide RNAs (gRNAs) targeting the endogenous copy of an essential gene, and
a second promoter operably linked to the nucleic acid encoding one or more guide RNAs (gRNAs),
and optionally one or more cargo sequences,
wherein the one or more vectors are positioned in a chromosome or an extra-chromosomal element.

66. The plant, plant part or plant seed of embodiment 65, wherein the vector renders a population of plants derived from the plant, plant part or plant seed vulnerable to toxicity in the presence of a chemical, compound or condition.

67. The plant, plant part or plant seed of embodiment 65, wherein the chemical or compound is an herbicide, pesticide or environmental condition such as temperature, salinity, light or water levels.

68. The plant, plant part or plant seed of embodiment 65, wherein the vector renders a population of plants derived from the plant, plant part or plant seed less harmful to other species in the environment.

69. The plant, plant part or plant seed of embodiment 65, wherein the gene spread renders a population of plants derived from the plant, plant part or plant seed resistant to toxicity in the presence of a chemical, compound or environmental condition.

70. The plant, plant part or plant seed of embodiment 65, wherein the gene spread results in suppression or elimination of a population of plants derived from the plant, plant part or plant seed.

71. The plant, plant part or plant seed of embodiment 65, wherein the plant is diploid or polyploid.

72. A method for population modification or suppression in a plant, the method comprising:
(i) obtaining the plant,
(ii) introducing into the plant during the plant's diploid stage, one or more vectors comprising a nucleic acid sequence encoding:
   a. a first gene encoding a rescue transgene;
   b. a first enhancer/promoter operably linked to a second gene encoding the DNA sequence modifying enzyme and optionally, one or more regulatory sequences;
   c. a second gene encoding a DNA sequence modifying enzyme;
   d. a second promoter operably linked to one or more genes encoding a guide RNA (gRNA) targeting the endogenous copy of an essential gene; and
   e. one or more genes encoding a guide RNA (gRNA) targeting the endogenous copy of an essential gene, and
   f. optionally, one or more cargo sequences,
(iii) expressing the DNA sequence modifying enzyme in a cell or cells of the plant before meiosis that ultimately gives rise to a germline such that the essential gene is rendered partially or wholly non-functional and
(iv) generating haploid gametes,
wherein a haploid gamete containing one or more vectors in which the essential gene has been rendered partially or wholly non-functional is rescued by the rescue transgene in the vector, and
wherein, a haploid gamete that fails to inherit the rescue transgene dies.

### EXAMPLES

### Example 1: Components of a ClvR-Based Gamete Killer Construct

The strength of a gene drive which is its ability to spread from low frequency and in the presence of significant fitness cost is increased when it biases inheritance in both sexes, something that is of particular importance when trying to bring about population suppression. Engineering ClvR-based gamete drive with this feature requires targeting a gene whose expression during the haploid stage is required for the survival and/or development of microgametophyte and megagametophyte (hereafter referred to as pollen (which contains sperm) and ovules [the ovule being a sporophytic structure in the ovary within which each megagametophyte, which includes the egg, develops]). Mutations in many ubiquitously expressed housekeeping genes (such as were targeted for LOF allele creation in insect ClvRs, likely have such a phenotype in plants given the extensive gene expression that occurs in gametes but are challenging to identify since such mutations cannot be passaged through the germline (Oberhofer et al. 2019; Oberhofer et al. 2020). Their identity is sometimes inferred by their absence in mutant collections (e.g. Berg et al. 2005). Alternatively, with the advent of methods for CRISPR-based mutagenesis, genes whose mutation results loss of male and female gametes can be identified through reverse genetics approaches that incorporate a rescuing transgene into the mutagenized genetic background (e.g. 37, 38). In this embodiment, one such gene, YKT61, a ubiquitously expressed R-SNARE protein involved in fusion between vesicle and target membranes is presented37.

The components that make up ClvR gamete killers in some embodiments are illustrated in FIG. 1A.

As a Rescue, a genomic fragment containing the Arabidopsis lyrata YKT61 gene (in which some amino acid coding region differences were recoded back to those of A. thaliana) was utilized. For the Cleaver, four gRNAs (SEQ ID NO:1-4) targeting conserved regions within the A. thaliana YKT61 coding sequence were expressed ubiquitously using individual U6 Pol-III promoters (Stuttmann et al. 2021). Several versions of Cas9 were tested. One lacked introns and carried a mutation (K918N) shown to increase Cas9 catalytic activity while a second one contained 13 introns, which were thought to increase expression (Hand et al. 2021; Grützner et al. 2021). Regulatory sequences from six different genes were used to drive Cas9 expression. Arabidopsis DMC1 is primarily expressed during meiotic stages (Klimyuk et al. 1997). Sequences from the CLAVATA3 (early stem cell identity (Fletcher et al. 1999)), APETALA1 (flower meristem identity (Mandel et al. 1992)) and AGAMOUS (reproductive floral organ primordia (Busch et al. 1999)) genes direct expression in adult sporophyte tissues that include the future germline. The CaMV35S46 and UBIQUITIN1047 promoters direct expression broadly, in many if not all cell types. The DMC1 promoter was used in combination with both versions of Cas9, while AGAMOUS, CLAVATA3, APETALA1, CaMV35S and UBIQUITIN10 sequences were used to direct expression of the version of Cas9 lacking introns.

The plasmids were assembled with standard molecular cloning techniques and Gibson assembly (Gibson et al. 2009). The gRNA cassette, composed of four repeats of gRNA with U6 promoters, was cloned with Golden Gate assembly. Enzymes utilized were obtained from NEB, and cloning as well as DNA extraction kits were sourced from Zymo. The A. lyrata Rescue gene was synthesized by Twist Bioscience. An intronized Cas9 variant known as zCas9i (pAGM55285, which was a gift from Sylvestre Marillonnet, Addgene #153212) was used (Grützner et al. 2021). In this Cas9 version, the RPS5 promoter was replaced with that of DMC1. Additionally, immediately upstream of the start codon, 21 base pairs from AT1G58420, which had previously been demonstrated to enhance translational efficiency was incorporated (Kim et al. 2014). The recoded A. lyrata Rescue was then incorporated into the construct. To assemble the gRNA cassette, shuttle vectors from Stuttmann et al (pDGE332, pDGE333, pDGE335, and pDGE336, which were a gift from Johannes Stuttmann, Addgene #153241, 153242, 153243, and 153244) were used (Stuttmann et al. 2021). Guides were designed in Benchling to target exon 1 (gRNA1 and 2), exon 2 (gRNA3) and exon 5 (gRNA4) of YKT61 and cloned into the Bbs1 digested shuttle vectors with annealed primers. The final Golden Gate assembly was performed with the Cas9-Rescue plasmid from above and Bsal. As no cleavage with the intronized Cas9 and the DMC1 promoter, as inferred by the Mendelian inheritance of the full ClvR construct in multiple transgenic lines was detected. Based on these results, the intronized Cas9 was replaced with one that had no introns but retained the NLS sequences at the termini (Cas9 without introns from pTX168, which was a gift from Daniel Voytas, Addgene #89257) (Tang et al. 2016). Additionally, a mutation (K918N) in the Cas9 sequence that was shown to enhance its catalytic activity was introduced (Hand et al. 2021). However, the DMC1-Cas9 version without introns also showed no evidence of cleavage. Based on the results obtained with other promoters (APETELA and UBIQUITIN 10) and this version of Cas9, it was inferred that the DMC1 promoter is likely to be relatively weak. For all additional promoters tested here, Cas9 version lacking introns and carrying the K918N mutation was used. Using Gibson assembly, the DMC1 promoter was replaced with transcriptional regulatory sequences from APETALA1, CLAVATA3, and AGAMOUS (chosen based on their efficacy in previous work utilizing a Cre/Lox reporter) (Van et al. 2009). Finally, two versions of ClvR utilizing regulatory sequences that drive more ubiquitous expression, from the UBIQUITIN10 gene and the CaMV35S promoter were built. Whole plasmid sequencing was performed by Plasmidsaurus, using Oxford Nanopore Technology with custom analysis and annotation.

A schematic representation of the behavior of ClvR in a diploid plant is illustrated in FIGS. 1B-1D.

### Example 2: ClvRs Based on Cleavage and Rescue of the YKT61 Gene Show Key Features Required for Gamete Killer Gene Drive

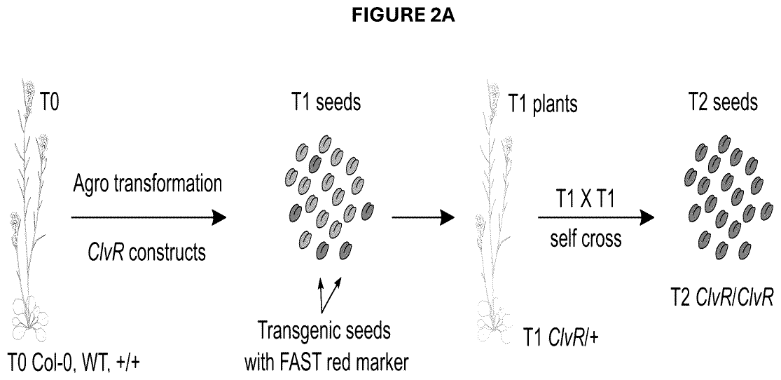

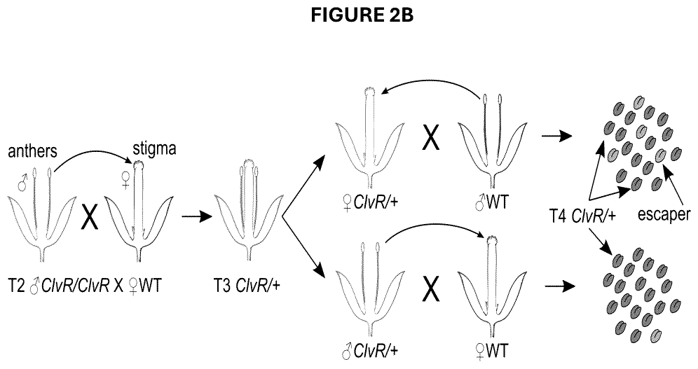

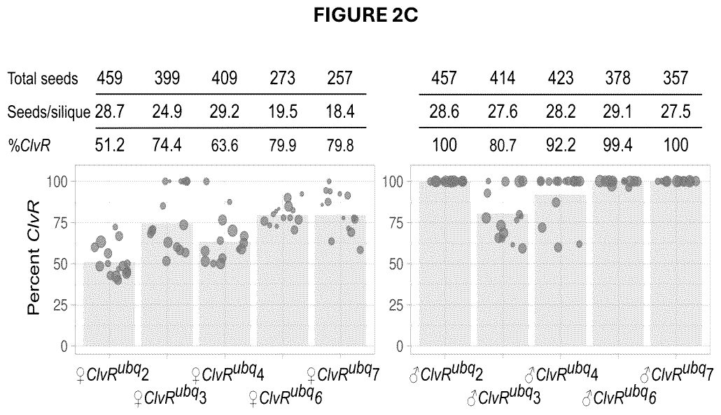

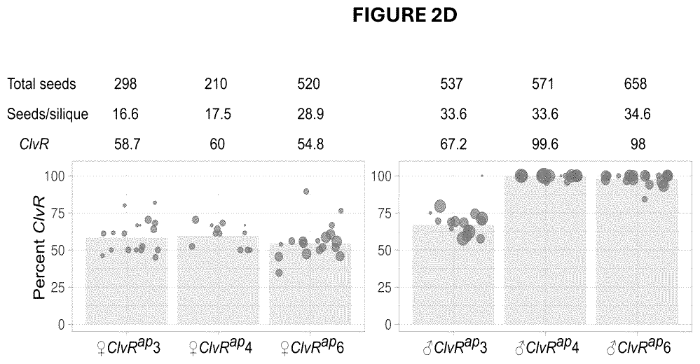

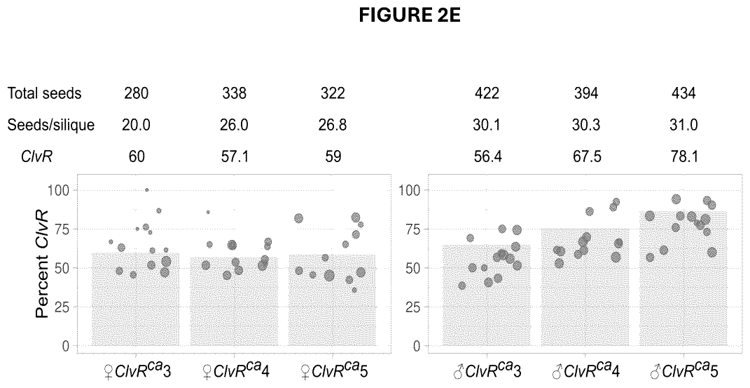

To test the system in a diploid system, a number of TO WT plants were transformed with the constructs discussed above using floral dipping with Agrobacterium as illustrated in FIG. 2A. All plants were grown in soil with a 16/8-hour light dark cycle. Temperature was 25° C. All seeds were planted directly in soil and stratified at 4° C. for 3 days. Transgenic plants were maintained in separate dedicated room in a hood mounted with a screen and surrounded by sticky tape, to minimize airflow around the plants and to prevent potential insect-mediated pollen movement. A floor-mounted sticky surface surrounding the hood performed a similar function. Transgenic plants were disposed of following autoclaving. Floral dip method with agrobacteria was used for transgenesis (Zhang et al. 2006). ClvR plasmids were transformed into GV3101 ElectroCompetent Agrobacterium strain from Intact Genomics. T1 seeds were screened for the FAST red seed marker and planted as described above (Shimada et al. 2010).

A number of independent transformants were collected from these plants and characterized in the crosses outlined in FIGS. 2A and 2B. T1 seeds (heterozygous for one or more ClvR elements) were grown to adulthood and allowed to self-cross (T1×T1). T1 self-crosses that produced progeny siliques (a seed pod, which contains progeny from the ovules of one flower) containing all or primarily red seeds—the T2 generation, possibly ClvR/CIvR homozygotes were characterized further as this is the expected phenotype if gametic drive occurred in one or both sexes in a cross between heterozygotes. Based on the results of these experiments (a significant fraction of non-ClvR seeds), constructs utilizing regulatory sequences from the DMC1, AGAMOUS, and CLAVATA-3 genes were not considered further.

T2 seeds carrying constructs that utilized the APETELA1 and UBIQUITIN10 regulatory sequences were grown to adulthood and pollen from these plants was used in an outcross to WT to produce T3 ClvR/+ seeds as illustrated in FIG. 2B. Finally, in the key outcross to test for gametic drive, T3 seeds were grown to adulthood and pollen and ovules used in outcrosses to WT. The frequency of ClvR inheritance (ClvR/+) in progeny T4 seeds provides a measure of gamete killing and rescue. ClvR inheritance rates in T4 seeds from T3 pollen, shown for 5 different insertions using UBIQUITIN10 sequences (ClvRubq lines) to direct Cas9 expression were generally very high, with three of the five showing inheritance rates greater than 99% as illustrated in FIG. 2C. Inheritance rates of ClvR in T4 seeds from T3 ovules were also significantly above 50%, but a number of non-ClvR seeds (generically referred to as escapers) were observed as illustrated in FIG. 2C. Similar results were obtained for ClvRs utilizing APETALA1 regulatory sequences (FIG. 2D). Crosses with ClvRs using the CaMV35S promoter showed inheritance that was modestly ClvR-biased (FIG. 2E). These results show that ClvRs designed to kill and rescue a gamete essential gene can bias inheritance in their favor in Arabidopsis, satisfying the key requirement for TA-based gametic gene drive.

T1 seeds were grown and allowed to self-cross. Siliques (seed pods, with each pod representing the fertilized ovules of a single ovary/flower) of these plants were screened for the FAST red marker again. The plants that showed 100% ClvR bearing seeds, suggesting drive activity (Mendelian genetics would result in 75% red seeds) were selected and it was observed that that ClvRdmc1, ClvRagamous, ClvRclavata3 had less than 100% red seeds in the self-crosses and decided not to further characterize these lines.

T2 ClvR seeds were grown and pollen from these plants was used in an outcross to WT females to generate heterozygous ClvR/+T3 seeds. T3 seeds were grown into adults again to set up reciprocal crosses with WT. ♀ClvR/+ for each line were crossed to ♂WT and ♀ClvR/+ were crossed to ♀WT (4 crosses per plant, 4 plants per line). Siliques of these crosses were scored for the ClvR marker as shown in FIG. 2A-2E.

### Example 3: ClvR-Based Gamete Killing and Rescue is Stable Over Multiple Generations

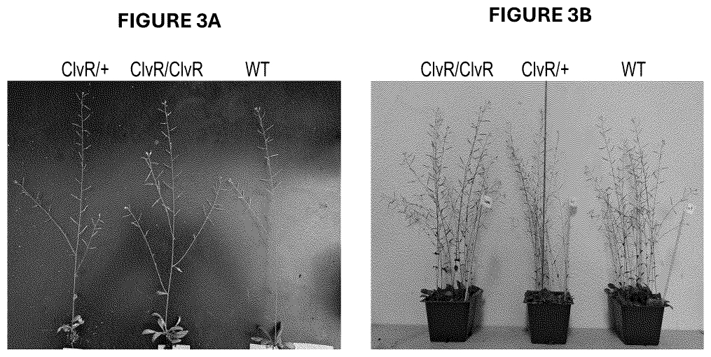

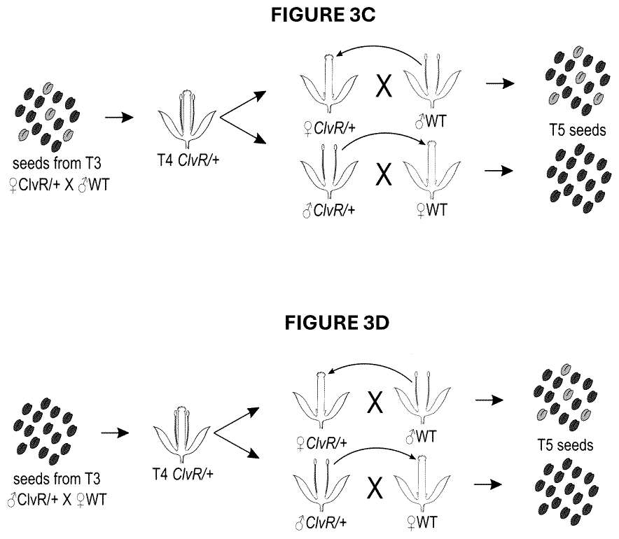

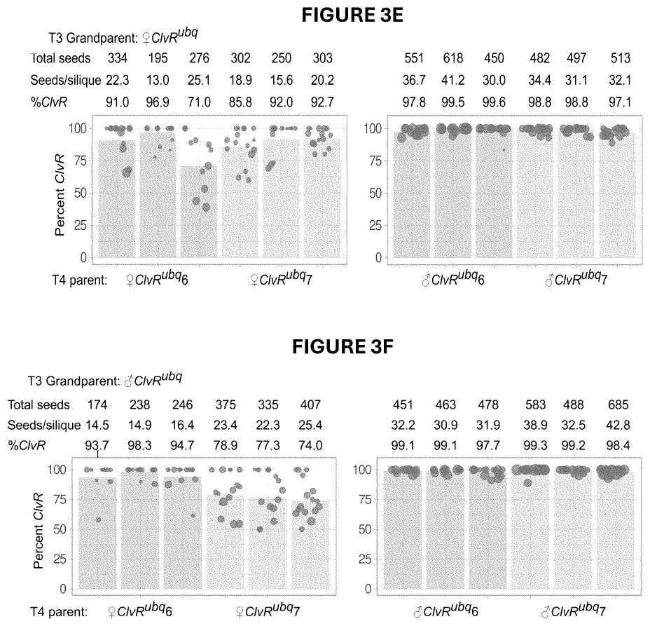

Next, ClvRubq6 and ClvRubq7 lines (generically referred to as ClvRubq) were characterized as these showed the highest frequency of inheritance through pollen and ovules, and heterozygotes and homozygotes were otherwise healthy as shown in FIGS. 3A and 3B. To determine if the bias in ClvRubq inheritance was stable, and if it had any dependence on the sex through which drive element was inherited, drive of ClvRubq alleles present in T4 ClvRubq/+ individuals that came from either a male or female ClvRubq/+T3 parent were characterized.

ClvRubq-bearing T4 seeds derived from male or female ClvRubq/+T3 parents crossed to WT were grown to adulthood and pollen and ovules from ClvRubq-bearing T4 individuals were used in outcrosses to WT, giving rise to a T5 generation of seeds whose ClvRubq/+ grandparents (the T3 generation) and parents (the T4 generation) were either both female (ovules), both male (pollen), or one (T3) and then the other (T4) as illustrated in FIG. 3C. T4 seeds from individual T3 crosses were taken and a set of reciprocal (male ClvR/+ to female WT and female ClvR/+ to male WT) crosses were repeated. 4 plants with 4 crosses per plant for each of 3 individual T3 crosses (results in FIG. 3) were crossed. Leaf tissue from T4 heterozygous ClvR plants and escapers from the ♀ClvR/+X ♂WT cross (non-ClvR bearing seeds) to extract DNA and sequence the YKT61 target sites were collected. Finally, T5 escaper seeds coming from: ♀ClvR/+ and from ♂ClvR/+ were grown. Leaves of young plants were again collected, and target sites sequenced. For sequencing, DNA from candidate plants was extracted from leaves with the Zymo Quick-DNA Plant/Seed miniprep kit according to the manufacturers protocol. The YKT61 target region was PCR amplified using primers ykt-cleaveF1 (TAGCATCTCCGAGTAAGGAATC (SEQ ID NO:5)) and ykt-cleaveR2 (CTTATAGATTTAGTTTCCTTTTTTCCCTGT (SEQ ID NO:6)). The PCR fragment was purified following agarose gel electrophoresis and sequenced by Plasmidsaurus at ˜1000× coverage. The resulting raw reads were mapped to the YKT61 reference using minimap2 (Li et al. 2018). The alignment file was sorted and indexed with samtools. The output file variants were then clustered with a Python script from Pacific Biosciences (https://github.com/PacificBiosciences/pbampliconclustering).

As shown in FIG. 3C-3F, inheritance rates remained comparable-very high when transmitted through pollen and high but with significantly more non-ClvRubq-bearing escaper seeds when transmitted through ovules-regardless of the parental and grandparental sex.

### Example 4: Mutations Associated with Cleavage and Molecular Basis of Escape from Gamete Killing

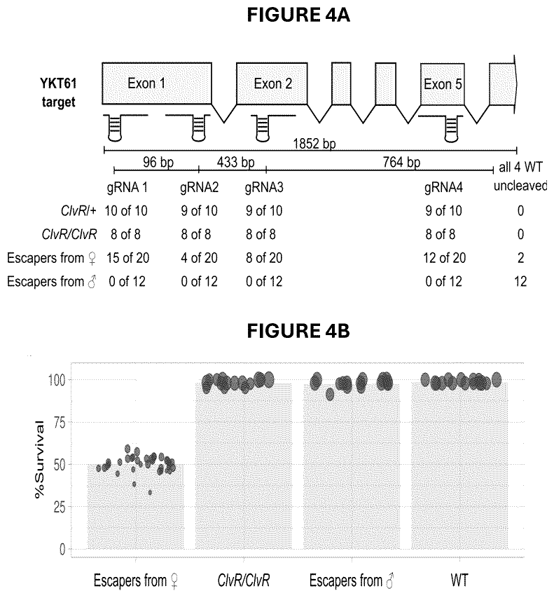

The high frequency of ClvRubq inheritance when transmitted through ClvRubq/+ pollen argues that rates of cleavage and LOF mutation creation are high, and that rescue is efficient. The UBIQUITIN10 regulatory sequences drive expression broadly throughout development, from the embryo onwards, long before the male and female germlines form (Geldner et al. 2009). This suggests that rates of cleavage and LOF allele creation in female gametes are high as well. To understand the molecular events associated with drive, and the unexpectedly high numbers of non-ClvRubq progeny observed when a ClvRubq/+ individual was the female parent, the endogenous YKT61 locus in leaves of several genotypes: ClvRubq/+T4 heterozygotes and ClvRubq/ClvRubq T5 homozygotes derived from a T4 self-cross were sequenced, with equal numbers of ClvRubq6 and ClvRubq7 sequenced for both crosses; and non-ClvRubq escapers from crosses of ClvRubq/+ to WT, in which the ClvRubq/+ was the female or male parent as illustrated in FIG. 4A-4B. In ClvRubq/+ individuals all four target sites were cleavable and mutated to LOF (frameshifts) at high frequency, with at least three sites being altered in all 10 plants. In ClvRubq/ClvRubq homozygotes all four sites were altered in all 8 sequenced individuals. In outcrosses using ClvRubq/+ pollen a very small number of non-ClvRubq escaper seeds was observed (˜1% of all seeds; as illustrated in FIG. 2A-2E, 3C-3F). Twelve T4 escapers were grown to adulthood and sequenced. All were WT at all four gRNA target sites as shown in FIG. 4A. Thus, escape from death by non-ClvRubq pollen is due to lack of cleavage and/or sequence alteration following cleavage, at all four sites. In contrast, in crosses with females as the parent 18 out of 20 non-ClvR progeny of a ClvRubq/+ parent carried one or more sequence alterations at gRNA target sites that create LOF alleles (frameshifts) in the YKT61 coding region as shown in FIGS. 4A and 4B. The other two escapers were wildtype at all four target sites. No resistant versions of endogenous YKT61—mutated but likely to be functional—were observed.

To summarize, rates of cleavage and mutation to LOF at the YKT61 locus were very high in male and female gametes. In males, death of non-ClvRubq pollen, coupled with efficient rescue of those inheriting ClvRubq, led to a very high frequency of ClvRubq inheritance in progeny. In females, inheritance of ClvRubq was also high, but there were also significant numbers of non-ClvRubq progeny. The results of sequencing showed that most of these have a LOF mutation, probably created by ClvRubq at a much earlier stage in the diploid. The construct was not present in plants grown from escaper seeds as illustrated in FIG. 4C-4E, arguing against transgene silencing playing a role. In earlier work, Cas9-induced LOF alleles of YKT61 were uniformly not passed to progeny through female gametes, resulting in abortion of 50% of ovules in a self-cross (Ma et al. 2021). Given this, our observations suggest that in the ClvRubq/+ diploid the YKT61 rescue transgene from A. lyrata provides YKT61 transcript and/or protein that is carried over from the mother into the non-ClvRubq haploid ovules (maternal carryover rescue), and that this is sufficient to rescue the survival of some gametes carrying a LOF YKT61 allele. A strong prediction of this hypothesis is that LOF alleles generated by ClvRubq and present in LOF/+, non-ClvRubq heterozygotes should, since they lack the ClvRubq Rescue, not be transmitted to the next generation. In a self-cross of female escapers (most of whom are LOF/+ heterozygotes based on the results of sequencing; FIG. 4)) this should manifest itself as 50% abortion in progeny siliques. As illustrated in FIG. 4B, this is the phenotype we observed for a number of female escapers tested.

The mechanism by which maternal ClvR rescues some female gametes from death requires further exploration. Recoding associated with use of A. lyrata YKT61 may have created an mRNA with an extended half-life. Alternatively, position effects based on chromatin structure and/or nearby transcriptional regulatory sequences may lead to increased expression and/or extend expression of A. lyrata YKT61 farther into meiosis, resulting in carryover into non-ClvR bearing gametes carrying a LOF mutation in YKT61. Incorporation of chromatin insulators (Kurbidaeva et al. 2021), and/or targeting of other essential genes can minimize such effects.

### Example 5: Modeling Predicts that ClvR Elements can Drive Rapid Population Modification and Suppression in Plants

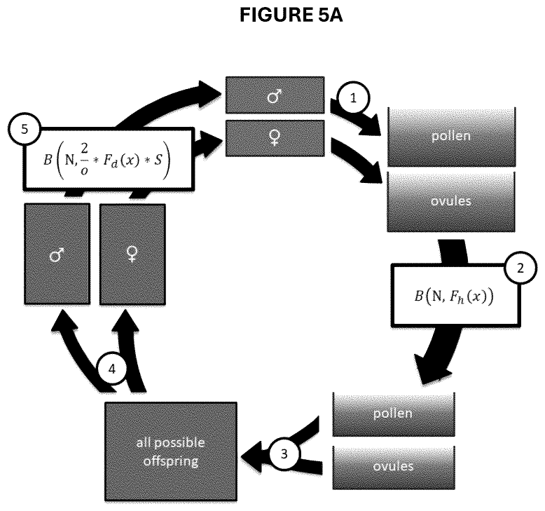

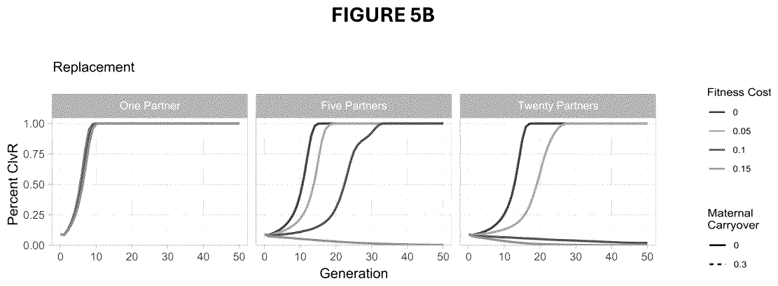

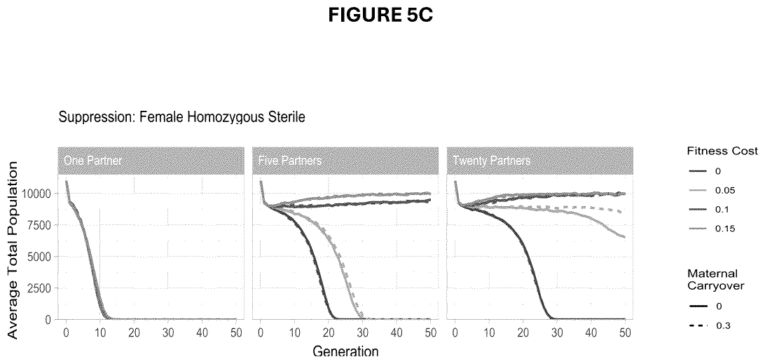

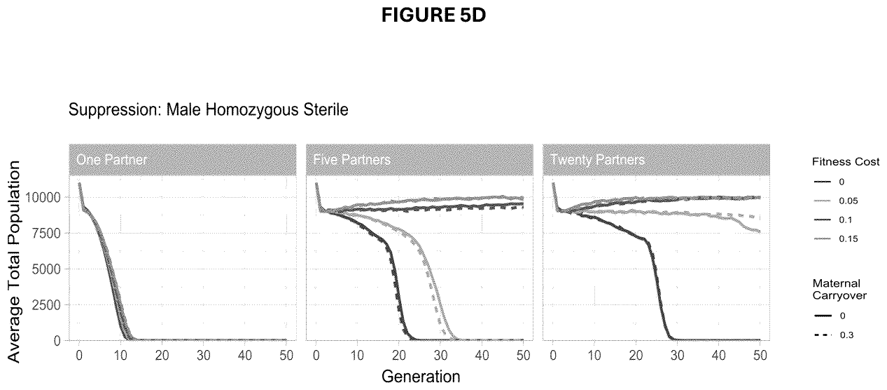

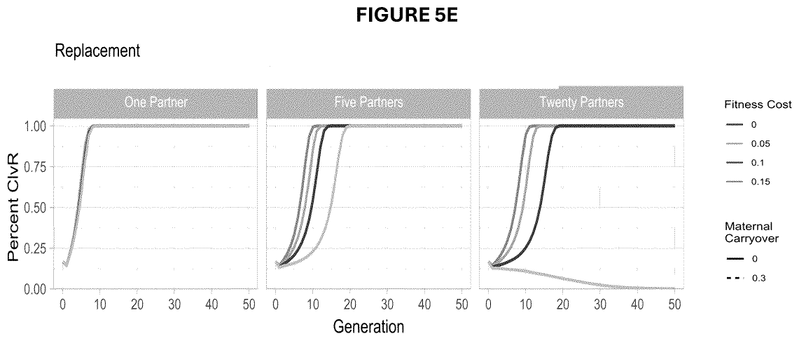

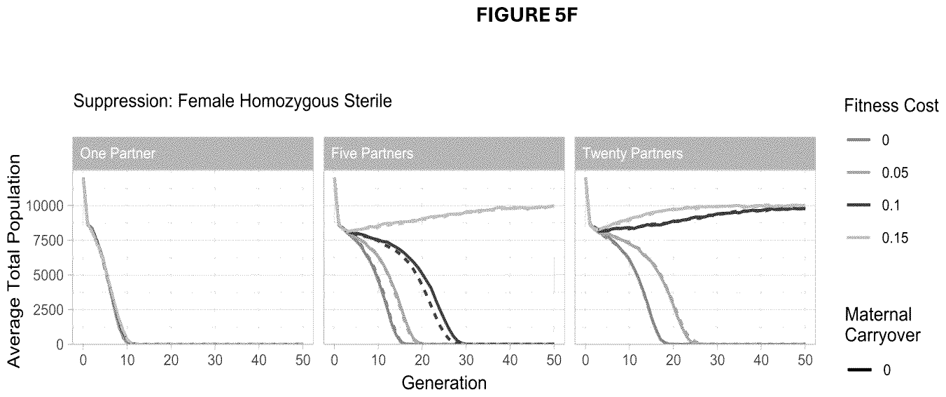

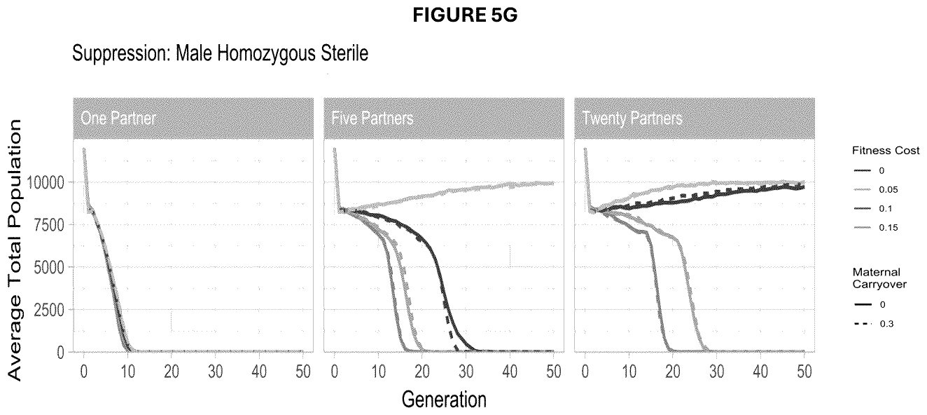

To explore the utility of ClvR-based gamete killers for population modification and suppression a stochastic model (panmictic population with non-overlapping generations that considers individual and gametes) was used to explore ClvR behavior in several scenarios. Modeling was performed using a stochastic agent-based model with discrete generations written in python. The model tracked and recorded dioecious diploid individuals in each generation and tracked gametes between generations as illustrated in FIG. 5A.

For the modeling process, each generation starts with a pool of adult individuals, shown at the top in purple. This information is stored as a list where each index represents a possible genotype, and the associated value represents the number of individuals of that genotype. (1) For each mating, a single mother produces a pool of ovules and multiple males produce a single pool of pollen. (2) The number of ovules and pollen are reduced, based on their haploid fitness costs Fₕ such that a pollen with no fitness costs will have a 100% chance of survival, and a pollen with fitness cost 0.1 will only have a 90% chance of surviving. (3) From the pool of possible pollen and ovules, each ovule is mated with a single pollen to produce a possible offspring. (4) The possible offspring are randomly grouped as male or female (5). The number of possible offspring is reduced to only those that survive. For each mating, the base chance of survival is 2 over the number of expected offspring o, as we expect each mating between a female and a male to produce 2 offspring when the population is at carrying capacity. This probability of survival is further modified by F_{d}, the fitness of the individual, and S. S denotes the density dependence function

$$S ( P ) = \frac{g}{1 + ( g - 1 ) \frac{P}{K}}$$

where P is the parent generation's population size, and K is the carrying capacity. S ranges from some growth factor g=6 at low densities, to 1 at densities near carrying capacity. The offspring that survive become the parents of the next generation.

For the data shown in FIG. 5B-5G, it was assumed that ClvR and the target gene were unlinked, that ClvR element had 95% efficiency in creating LOF alleles, and that the population had a low-density growth rate of 6. More information on the model, the scripts and parameters used to generate the data, and the data itself can be found on https://github.com/MicycleJ/Pigss/tree/main

In animals, gene drive is often modeled using a paradigm in which matings are monogamous and sperm is not limiting. However, in many plants of interest (crops, weeds, targets of conservation) polyandry (fertilization of a female with pollen sourced from multiple males, which is in excess) is likely to be more relevant (Pannell et al. 2013). In other contexts not explored here, pollen limitation can occur (Knight et al. 2005). The mating system is important to consider since the relative benefit in transmission frequency that ClvR-bearing gametes gain in fertilizing ovules, due to loss of competing non-ClvR gametes, decreases as the number of competing non-ClvR gametes from other males increases. Here some representative examples of outcomes when a pan-gamete killing ClvR is introduced into a population and the mating system is monogamous is provided—a best-case scenario in which ClvR gametes from a single male monopolize the ovary of a female—or polyandrous, with 5 or 20 males each contributing ⅕ or 1/20 of their pollen to a female. The role of gamete fitness costs, as might arise due to incomplete rescue or cleavage-induced aneuploidy that manifests itself as death during the haploid stage is also considered (Kosicki et al. 2018). The effects of maternal carryover of Rescue activity are also considered.

Population modification was first considered. FIG. 5B-D shows examples in which ClvR is introduced at a frequency of 10% into a WT population. The LOF allele creation rate is set to 95%, somewhat lower than the rate inferred from the results presented earlier in FIG. 3C-3F and FIG. 4A-4B). Gametic fitness costs (dominant because they are in the haploid stage) varied between 0% and 15%. Maternal carryover was set to zero as exploration of other scenarios shows it has very little impact on population modification. With monogamous mating ClvR spreads rapidly over a range of fitness costs as illustrated in FIG. 5B. In the presence of polyandry, drive is slowed and fails to spread for some higher fitness costs as shown in FIG. 5C-5D. However, drive can be restored if the introduction frequency is increased as shown in FIG. 5E-5G. When drive occurs, the ClvR spreads to allele fixation. This is because whenever a WT non-ClvR homologous chromosome is present with ClvR (and the LOF allele creation rate is high) it has a very high probability of being eliminated from the viable gamete pool since it lacks a Rescue transgene. Strong but self-limiting drive can presumably also be achieved by locating Cas9 (or gRNAs) at some distance from the other ClvR components, as with ClvRs that kill specific zygote genotypes (Oberhofer et al. 2021).

For population suppression two scenarios were considered, in which ClvR is located in a gene (thereby disrupting it) whose recessive LOF in the sporophyte results in female (FIG. 5C) or male (FIG. 5D) infertility (Champer et al. 2020). As above, gamete killing and rescue occurs in both sexes and some level of maternal carryover rescue of LOF allele-bearing gametes may be present. In both scenarios a gamete killer can drive the population towards a homozygous male or female sterile state, resulting in population extinction. Maternal carryover has a very modest effect on drive towards a homozygous female sterile state because it allows some WT alleles to persist for some time in non-ClvR progeny of a ClvR-bearing mother. However, it has no effect on drive towards a homozygous male sterile state. As with population modification, fitness costs can slow or prevent drive and suppression but increasing the introduction frequency can restore drive to the homozygous sterile state as shown in FIG. 5E-5G.

### REFERENCES

1. Hay, B. A., Oberhofer, G. & Guo, M. Engineering the Composition and Fate of Wild Populations with Gene Drive. Annu. Rev. Entomol. 66, 407-434 (2021).
2. Bier, E. Gene drives gaining speed. Nat Rev Genet23, 5-22 (2022).
3. Raban, R., Marshall, J. M., Hay, B. A. & Akbari, O. S. Manipulating the Destiny of Wild Populations Using CRISPR. Annual Review of Genetics57, null (2023).
4. Burga, A., Ben-David, E. & Kruglyak, L. Toxin-Antidote Elements Across the Tree of Life. Annual review of genetics54, 387-415 (2020).
5. De Carvalho, M. et al. The wtf meiotic driver gene family has unexpectedly persisted for over 100 million years. eLife11, e81149 (2022).
6. Wang, C. et al. A natural gene drive system confers reproductive isolation in rice. Cell 186, 3577-3592.e18 (2023).
7. Jurėnas, D., Fraikin, N., Goormaghtigh, F. & Van Melderen, L. Biology and evolution of bacterial toxin-antitoxin systems. Nat Rev Microbiol20, 335-350 (2022).
8. Hamilton, W. D. Extraordinary sex ratios. A sex-ratio theory for sex linkage and inbreeding has new implications in cytogenetics and entomology. Science 156, 477-88 (1967).
9. Wade, M. J. & Beeman, R. W. The population dynamics of maternal-effect selfish genes. Genetics138, 1309-14 (1994).
10. Hastings, I. M. Selfish DNA as a method of pest control. Philos. Trans. R. Soc. Lond. B Biol. Sci. 344, 313-324 (1994).
11. Smith, N. G. The dynamics of maternal-effect selfish genetic elements. J Theor Biol191, 173-80 (1998).
12. Ward, C. M. et al. Medea selfish genetic elements as tools for altering traits of wild populations: a theoretical analysis. Evolution65, 1149-1162 (2011).
13. Marshall, J. M. & Hay, B. A. General principles of single-construct chromosomal gene drive. Evolution66, 2150-66 (2012).
14. Oberhofer, G., Ivy, T. & Hay, B. A. Cleave and Rescue, a novel selfish genetic element and general strategy for gene drive. Proc Natl Acad Sci USA 116, 6250-6259 (2019).
15. Champer, J. et al. A toxin-antidote CRISPR gene drive system for regional population modification. Nature communications11, 1082 (2020).
16. Champer, J., Kim, I. K., Champer, S. E., Clark, A. G. & Messer, P. W. Performance analysis of novel toxin-antidote CRISPR gene drive systems. BMC biologyl8, 27 (2020).
17. Martinossi-Allibert, I. et al. Invasion and maintenance of meiotic drivers in populations of ascomycete fungi. Evolution75, 1150-1169 (2021).
18. Li, J. & Champer, J. Harnessing Wolbachia cytoplasmic incompatibility alleles for confined gene drive: A modeling study. PLOS Genet19, e1010591 (2023).
19. Chen, C. H. et al. A synthetic maternal-effect selfish genetic element drives population replacement inDrosophila. Science 316, 597-600 (2007).
20. Akbari, O. S. et al. Novel synthetic Medea selfish genetic elements drive population replacement in Drosophila; a theoretical exploration of Medea-dependent population suppression. ACS Synth Biol3, 915-28 (2014).
21. Buchman, A., Marshall, J. M., Ostrovski, D., Yang, T. & Akbari, O. S. Synthetically engineered Medea gene drive system in the worldwide crop pest Drosophila suzukii. Proc Natl Acad Sci USA 115, 4725-4730 (2018).
22. Oberhofer, G., Ivy, T. & Hay, B. A. Gene drive and resilience through renewal with next generation Cleave and Rescues elfish genetic elements. Proc Natl Acad Sci USA 117, 9013-9021 (2020).
23. Oberhofer, G., Ivy, T. & Hay, B. A. Split versions of Cleave and Rescue selfish genetic elements for measured self limiting gene drive. PLOS Genet17, e1009385 (2021).
24. Oberhofer, G., Ivy, T. & Hay, B. A. Gene drive that results in addiction to a temperature-sensitive version of an essential gene triggers population collapse in Drosophila. Proc Natl Acad Sci USA118, e2107413118 (2021).
25. Metzloff, M. et al. Experimental demonstration of tethered gene drive systems for confined population modification or suppression. BMC Biol20, 119 (2022).
26. Champer, J., Champer, S. E., Kim, I. K., Clark, A. G. & Messer, P. W. Design and analysis of CRISPR-based under dominance toxin-antidote gene drives. Evolutionary Applications14, 1052-1069 (2021).
27. Bhutani, K. et al. Widespread haploid-biased gene expression enables sperm-level natural selection. Science 371, eabb1723 (2021).
28. Immler, S. Haploid Selection in “Diploid” Organisms. Annu. Rev. Ecol. Evol. Syst. 50, 219-236 (2019).
29. Somers, J. & Nelms, B. The sporophyte-to-gametophyte transition: The haploid generation comes of age. Current Opinion in Plant Biology75, 102416 (2023).
30. Howden, R. et al. Selection of T-DNA-Tagged Male and Female Gametophytic Mutants by Segregation Distortion in Arabidopsis. Genetics149, 621-631 (1998).
31. Christensen, C. A., Subramanian, S. & Drews, G. N. Identification of Gametophytic Mutations Affecting Female Gametophyte Development in Arabidopsis. Developmental Biology202, 136-151 (1998).
32. Abbott, R. J. & Gomes, M. F. Population genetic structure and outcrossing rate of Arabidopsis thaliana (L.) Heynh. Heredity62, 411-418 (1989).
33. Hoffmann, M. H. et al. Flower visitors in a natural population of Arabidopsis thaliana. Plant Biology5, 491-494 (2003).
34. Mercier, R. et al. Outcrossing as an explanation of the apparent unconventional genetic behavior of Arabidopsis thaliana hth mutants. Genetics180, 2295-7 (2008).
35. Tan, Y. Y. et al. Transgenic GFP as a molecular marker for approaches to quantify pollination mechanism and gene flow in Arabidopsis thaliana. Plant Biol (Stuttg) 7, 405-10 (2005).
36. Berg, M., Rogers, R., Muralla, R. & Meinke, D. Requirement of aminoacyl-tRNA synthetases for gametogenesis and embryo development in Arabidopsis. The Plant journal: for cell and molecular biology44, 866-78 (2005).
37. Ma, T., Li, E., Li, L., Li, S. & Zhang, Y. The Arabidopsis R-SNARE protein YKT61 is essential for gametophyte development. JIPB63, 676-694 (2021).
38. Liu, F. et al. The canonical α-SNAP is essential for gametophytic development in Arabidopsis. PLOS Genet17, e1009505 (2021).
39. Stuttmann, J. et al. Highly efficient multiplex editing: one-shot generation of 8× Nicotiana benthamiana and 12× Arabidopsis mutants. The Plant Journal 106, 8-22 (2021).
40. Hand, T. H. et al. Catalytically Enhanced Cas9 Through Directed Protein Evolution. The CRISPR Journal 4, 223-232 (2021).
41. Grützner, R. et al. High-efficiency genome editing in plants mediated by a Cas9 gene containing multiple introns. Plant Communications2, 100135 (2021).
42. Klimyuk, V. I. & Jones, J. D. AtDMC1, the Arabidopsis homologue of the yeast DMC1 gene: characterization, transposon-induced allelic variation and meiosis-associated expression. The Plant journal: for cell and molecular biology11, 1-14 (1997).
43. Fletcher, J. C., Brand, U., Running, M. P., Simon, R. & Meyerowitz, E. M. Signaling of Cell Fate Decisions by CLAVATA3 in Arabidopsis Shoot Meristems. Science 283, 1911-1914 (1999).
44. Alejandra Mandel, M., Gustafson-Brown, C., Savidge, B. & Yanofsky, M. F. Molecular characterization of the Arabidopsis floral homeotic gene APETALA1. Nature360, 273-277 (1992).
45. Busch, M. A., Bomblies, K. & Weigel, D. Activation of a Floral Homeotic Gene in Arabidopsis. Science 285, 585-587 (1999).
46. Amack, S. C. & Antunes, M. S. CaMV35S promoter-A plant biology and biotechnology workhorse in the era of synthetic biology. Current Plant Biology24, 100179 (2020).
47. Norris, S. R., Meyer, S. E. & Callis, J. The intron of Arabidopsis thaliana polyubiquitin genes is conserved in location and is a quantitative determinant of chimeric gene expression. Plant Mol Biol21, 895-906 (1993).
48. Geldner, N. et al. Rapid, combinatorial analysis of membrane compartments in intact plants with a multicolor marker set. The Plant Journal 59, 169-178 (2009).
49. Kurbidaeva, A. & Purugganan, M. Insulators in Plants: Progress and Open Questions. Genes12, 1422 (2021).
50. Pannell, J. R. & Labouche, A.-M. The incidence and selection of multiple mating in plants. Phil. Trans. R. Soc. B368, 20120051 (2013).
51. Knight, T. M. et al. Pollen Limitation of Plant Reproduction: Pattern and Process. Annu. Rev. Ecol. Evol. Syst. 36, 467-497 (2005).
52. Kosicki, M., Tomberg, K. & Bradley, A. Repair of double-strand breaks induced by CRISPR-Cas9 leads to large deletions and complex rearrangements. Nat Biotechnol36, 765-771 (2018).
53. Chaudhury, A. M. Nuclear Genes Controlling Male Fertility. Plant Cell 1277-1283 (1993) doi: 10.1105/tpc. 5.10.1277.
54. Modrusan, Z., Reiser, L., Feldmann, K. A., Fischer, R. L. & Haughn, G. W. Homeotic Transformation of Ovules into Carpel-like Structures in Arabidopsis. Plant Cell 333-349 (1994) doi: 10.1105/tpc. 6.3.333.
55. Reiser, L. et al. The BELL1 gene encodes a homeodomain protein involved in pattern formation in the Arabidopsis ovule primordium. Cell 83, 735-742 (1995).
56. Wilson, Z. A., Morroll, S. M., Dawson, J., Swarup, R. & Tighe, P. J. The Arabidopsis MALE STERILITY1 (MS1) gene is a transcriptional regulator of male gametogenesis, with homology to the PHD-finger family of transcription factors: MS1 a transcriptional regulator of male gametogenesis. The Plant Journal 28, 27-39 (2001).
57. Montgomery, J. S., Giacomini, D. A., Weigel, D. & Tranel, P. J. Male-specific Y-chromosomal regions in waterhemp (Amaranthus tuberculatus) and Palmer amaranth (Amaranthus palmeri). New Phytologist229, 3522-3533 (2021).
58. Leite Montalvão, A. P., Kersten, B., Fladung, M. & Müller, N. A. The Diversity and Dynamics of Sex Determination in Dioecious Plants. Front. Plant Sci. 11, 580488 (2021).
59. Lyttle, T. W. Experimental population genetics of meiotic drive systems. I. Pseudo-Y chromosomal drive as a means of eliminating cage populations of Drosophila melanogaster. Genetics86, 413-45 (1977).
60. Heinrich, J. C. & Scott, M. J. A repressible female-specific lethal genetic system for making transgenic insect strains suitable for a sterile-release program. Proc Natl Acad Sci USA 97, 8229-32 (2000).
61. Thomas, D. D., Donnelly, C. A., Wood, R. J. & Alphey, L. S. Insect Population Control Using a Dominant, Repressible, Lethal Genetic System. Science 287, 2474-2476 (2000).
62. Schliekelman, P. & Gould, F. Pest control by the introduction of a conditional lethal trait on multiple loci: potential, limitations, and optimal strategies. J Econ Entomol93, 1543-65 (2000).
63. Isaac, R. S. et al. Nucleosome breathing and remodeling constrain CRISPR-Cas9 function. eLife5, e13450 (2016).
64. Horlbeck, M. A. et al. Nucleosomes impede Cas9 access to DNA in vivo and in vitro. eLife5, e12677 (2016).
65. Yarrington, R. M., Verma, S., Schwartz, S., Trautman, J. K. & Carroll, D. Nucleosomes inhibit target cleavage by CRISPR-Cas9 in vivo. Proc Natl Acad Sci USA 115, 9351-9358 (2018).
66. Heslop-Harrison, J. S. (Pat), Schwarzacher, T. & Liu, Q. Polyploidy: its consequences and enabling role in plant diversification and evolution. Annals of Botanyl31, 1-10 (2023).
67. Jones, D. L. et al. Kinetics of dCas9 target search in Escherichia coli. Science 357, 1420-1424 (2017).
68. Clark, M. & Maselko, M. Transgene Biocontainment Strategies for Molecular Farming. Front. Plant Sci. 11, 210 (2020).
69. Neve, P. Gene drive systems: do they have a place in agricultural weed management? Pest Management Science 74, 2671-2679 (2018).
70. Barrett, L. G. et al. Gene drives in plants: opportunities and challenges for weed control and engineered resilience. Proc. R. Soc. B. 286, 20191515 (2019).
71. Roberts, J. & Florentine, S. A review of the biology, distribution patterns and management of the invasive species Amaranthus palmeri S. Watson (Palmer amaranth): Current and future management challenges. Weed Research 62, 113-122 (2022).
72. Koo, D.-H. et al. Extrachromosomal circular DNA-based amplification and transmission of herbicide resistance in crop weed Amaranthus palmeri. Proc. Natl. Acad. Sci. U.S.A. 115, 3332-3337 (2018).
73. Molin, W. T., Yaguchi, A., Blenner, M. & Saski, C. A. The EccDNA Replicon: A Heritable, Extranuclear Vehicle That Enables Gene Amplification and Glyphosate Resistance in Amaranthus palmeri. The Plant Cell 32, 2132-2140 (2020).
74. Gibson, D. G. et al. Enzymatic assembly of DNA molecules up to several hundred kilobases. Nat. Methods6, 343-345 (2009).
75. Grützner, R. et al. High-efficiency genome editing in plants mediated by a Cas9 gene containing multiple introns. Plant Commun2, 100135 (2021).
76. Kim, Y. et al. The immediate upstream region of the 5′-UTR from the AUG start codon has a pronounced effect on the translational efficiency in Arabidopsis thaliana. Nucleic Acids Res42, 485-498 (2014).
77. Stuttmann, J. et al. Highly efficient multiplex editing: one-shot generation of 8× Nicotiana benthamiana and 12× Arabidopsis mutants. The Plant Journal 106, 8-22 (2021).
78. Tang, X. et al. A Single Transcript CRISPR-Cas9 System for Efficient Genome Editing in Plants. Mol Plant9, 1088-1091 (2016).
79. Hand, T. H. et al. Catalytically Enhanced Cas9 Through Directed Protein Evolution. CRISPR J4, 223-232 (2021).
80. Van Ex, F., Verweire, D., Claeys, M., Depicker, A. & Angenon, G. Evaluation of seven promoters to achieve germline directed Cre-lox recombination in Arabidopsis thaliana. Plant Cell Rep28, 1509-1520 (2009).
81. Zhang, X., Henriques, R., Lin, S.-S., Niu, Q.-W. & Chua, N.-H. Agrobacterium-mediated transformation of Arabidopsis thaliana using the floral dip method. Nat Protoc1, 641-646 (2006).
82. Shimada, T. L., Shimada, T. & Hara-Nishimura, I. A rapid and non-destructive screenable marker, FAST, for identifying transformed seeds of Arabidopsis thaliana. The Plant Journal 61, 519-528 (2010).
83. Li, H. Minimap2: pairwise alignment for nucleotide sequences. Bioinformatics34, 3094-3100 (2018).
84. Braun, R. E., Behringer, R. R., Peschon, J. J., Brinster, R. L., Palmiter, R. D. Genetically haploid spermatids are phenotypically diploid. Nature 337, 373-376 (1989).
85. Hime, G. R., Brill, J. A., Fuller, M. T. Assembly of ring canals in the male germ line from structural components of the contractile ring. J Cell Sci 109, 2779-2788 (1996).
86. Niedenberger, B. A. et al., Dynamic cytoplasmic projections connect mammalian spermatogonia in vivo. Development 145, dev161323 (2018).

What is claimed is:

1. A method for performing spread of a gene or multi-gene cassette in a population of an organism, comprising:
(i) obtaining the organism,
(ii) introducing into the organism one or more vectors comprising a nucleic acid sequence encoding:
a. a first gene encoding a rescue transgene;
b. a first enhancer/promoter operably linked to a second gene encoding the DNA sequence modifying enzyme and optionally, one or more regulatory sequences;
c. a second gene encoding a DNA sequence modifying enzyme;
d. a second promoter operably linked to one or more genes encoding a guide RNA (gRNA) targeting the endogenous copy of an essential gene; and
e. one or more genes encoding a guide RNA (gRNA) targeting the endogenous copy of an essential gene, and
f. optionally, one or more cargo sequences,
(iii) expressing the DNA sequence modifying enzyme in a cell or cells of the organism that ultimately gives rise to a germline such that the essential gene is rendered partially or wholly non-functional,
(iv) generating an altered gamete containing the one or more vectors, wherein the altered gamete in which the essential gene has been rendered partially or wholly non-functional has been rescued by the rescue transgene in the one or more vectors, and
(v) introducing a progeny carrying the altered gamete in an environment to generate a population of the organism, wherein progeny carrying the vector comprising the rescue gene increases in frequency relative to a frequency of a wild type individual organism in the population of the organism.

2. The method of claim 1, wherein the one or more vectors are configured such that the second gene, the first enhancer/promoter, the second promoter and/or the nucleic acid encoding one or more gRNAs are positioned on a different chromosome or an extra-chromosomal element than the first gene.

3. The method of claim 1, wherein the one or more vectors are configured such that the second gene, the first enhancer/promoter, the second promoter and/or the nucleic acid encoding one or more gRNAs are positioned on the same chromosome at a distance from the first gene.

4. The method of claim 1, wherein the organism is a diploid that produces haploid gametes.

5. The method of claim 1, wherein the organism is a plant.

6. The method of claim 1, wherein the gene spread renders the population of an organism vulnerable to toxicity in the presence of a chemical, compound or condition.

7. The method of claim 6, wherein the chemical or compound is an herbicide, pesticide or environmental condition such as temperature, salinity, light or water levels.

8. The method of claim 6, wherein the gene spread renders the population of an organism less harmful to other species in the environment.

9. The method of claim 1, wherein the gene spread renders the population of an organism resistant to toxicity in the presence of a chemical, compound or environmental condition.

10. The method of claim 9, wherein the condition is drought, stress, inadequate soil fertility, nutrient deficiencies, salinity, pollution, flooding, or presence of a pest, competitor, or pathogen.

11. The method of performing a gene drive-mediated population suppression of an organism comprising:
(i) obtaining the organism,
(ii) introducing into the organism one or more vectors comprising a nucleic acid sequence encoding:
a. a first gene encoding a rescue transgene;
b. a first enhancer/promoter operably linked to a second gene encoding the DNA sequence modifying enzyme and optionally, one or more regulatory sequences;
c. a second gene encoding a DNA sequence modifying enzyme;
d. a second promoter operably linked to one or more genes encoding a guide RNA (gRNA) targeting the endogenous copy of an essential gene; and
e. one or more genes encoding a guide RNA (gRNA) targeting the endogenous copy of an essential gene, and
f. optionally, one or more cargo sequences,
(iii) inserting the one or more vectors into a gene required for diploid fertility of one sex, thereby disrupting the gene required for diploid fertility of one sex,
(iv) expressing the DNA sequence modifying enzyme in a cell of the organism that ultimately gives rise to a germline, such that the essential gene is rendered partially or wholly non-functional,
(v) generating an altered gamete containing one or more vectors, wherein the altered gamete in which the essential gene has been rendered partially or wholly non-functional has been rescued by the rescue transgene in the one or more vectors, and
(vi) introducing a progeny carrying the altered gamete containing one or more vectors in a population of the organism, wherein gametes that do not contain the one or more vectors do not survive such that there is a relative increase in frequency of progeny comprising the vector in a population of the organism, and wherein those progeny are sterile due to the disruption of the gene required for diploid fertility.

12. The method of claim 11, wherein the essential gene is required for gamete function.

13. The method of claim 11, wherein the organism gives rise to a haploid gamete.

14. The method of claim 11, wherein the organism is a plant.

15. The method of performing a gene drive-mediated population suppression of an organism comprising:
(i) obtaining the organism,
(ii) introducing into the organism one or more vectors comprising a nucleic acid sequence encoding:
a. a first gene encoding a rescue transgene;
b. a first enhancer/promoter operably linked to a second gene encoding the DNA sequence modifying enzyme and optionally, one or more regulatory sequences;
c. a second gene encoding a DNA sequence modifying enzyme,
d. a second promoter operably linked to one or more genes encoding a guide RNA (gRNA) targeting the endogenous copy of an essential gene; and
e. one or more genes encoding a guide RNA (gRNA) targeting the endogenous copy of an essential gene, and
f. optionally, one or more cargo sequences,
wherein the one or more vectors is inserted next to or within a locus whose presence is sufficient for adult male or female sex determination,
(iii) expressing the DNA sequence modifying enzyme in a cell of the organism that ultimately gives rise to a germline, inducing one or more sequence modifications in the cell, such that the one or more sequence modifications result in the essential gene being rendered partially or wholly non-functional,
(iv) generating an altered gamete containing one or more vectors, wherein the altered gamete in which the essential gene has been rendered partially or wholly non-functional has been rescued by the rescue transgene in the vector, wherein the progeny carrying the vector comprising the rescue gene survive such that they increase in frequency, and
(v) introducing a progeny carrying the altered gamete containing one or more vectors in a population of the organism.

16. The method of claim 15, wherein the vector is linked to a male determining locus and gives rise to male, but not female, progeny.

17. The method of claim 15, wherein the vector is linked to a female determining locus and gives rise to female, but not male, progeny.

18. The method of claim 15, wherein the organism is a plant.

19. The method of claim 1, wherein the DNA sequence modifying enzyme is selected from the group consisting of Cas9, Cas-9-related RNA-guided nucleases, ZFN, TALEN, homing endonuclease, natural site-specific nucleases, engineered site-specific nucleases, prime editing enzymes, transposases, base editing enzymes, cytidine deaminase, and adenine deaminase.

20. A plant, a plant part or a plant seed comprising one or more vectors, wherein the one or more vectors that comprise:
a first gene encoding a rescue transgene;
a second gene encoding a DNA sequence modifying enzyme, wherein the DNA modifying enzyme modifies an endogenous copy of an essential gene;
a first enhancer/promoter operably linked to the second gene encoding the DNA sequence modifying enzyme and optionally, one or more regulatory sequences;
a nucleic acid encoding one or more guide RNAs (gRNAs) targeting the endogenous copy of an essential gene, and
a second promoter operably linked to the nucleic acid encoding one or more guide RNAs (gRNAs),
and optionally one or more cargo sequences,
wherein the one or more vectors is configured to be positioned in a chromosome or an extra-chromosomal element.

21. A method for population modification or suppression in a plant, the method comprising:
(i) obtaining the plant,
(ii) introducing into the plant during the plant's diploid stage, one or more vectors comprising a nucleic acid sequence encoding:
a. a first gene encoding a rescue transgene;
b. a first enhancer/promoter operably linked to a second gene encoding the DNA sequence modifying enzyme and optionally, one or more regulatory sequences;
c. a second gene encoding a DNA sequence modifying enzyme;
d. a second promoter operably linked to one or more genes encoding a guide RNA (gRNA) targeting the endogenous copy of an essential gene; and
e. one or more genes encoding a guide RNA (gRNA) targeting the endogenous copy of an essential gene, and
f. optionally, one or more cargo sequences,
(iii) expressing the DNA sequence modifying enzyme in a cell or cells of the plant before meiosis that ultimately gives rise to a germline such that the essential gene is rendered partially or wholly non-functional and
(iv) generating haploid gametes,
wherein a haploid gamete containing one or more vectors in which the essential gene has been rendered partially or wholly non-functional is rescued by the rescue transgene in the vector, and
wherein, a haploid gamete that fails to inherit the rescue transgene dies.


SEQUENCE LISTING (6 sequences)

SEQ ID NO: 1 - RNA, 20 nt, synthetic construct
  source 1..20: mol_type = other RNA
tgtgaagttttggttgtttc

SEQ ID NO: 2 - RNA, 20 nt, synthetic construct
  source 1..20: mol_type = other RNA
ttttcaaagatcttctgtta

SEQ ID NO: 3 - RNA, 20 nt, synthetic construct
  source 1..20: mol_type = other RNA
ctctttttcgtctagattgt

SEQ ID NO: 4 - RNA, 20 nt, synthetic construct
  source 1..20: mol_type = other RNA
agaaaagttggattctttgg

SEQ ID NO: 5 - DNA, 22 bp, synthetic construct
  source 1..22: mol_type = other DNA
tagcatctccgagtaaggaatc

SEQ ID NO: 6 - DNA, 30 bp, synthetic construct
  source 1..30: mol_type = other DNA
cttatagatttagtttccttttttccctgt